SEQUENCE LISTING (331 sequences)

SEQ ID NO: 1 - protein, 8 aa, Synthetic construct
  source 1..8: note = Encoded peptide
KDHDIDYK

SEQ ID NO: 2-5 - skipped (4 entries)

SEQ ID NO: 6 - RNA, 4204 nt, Synthetic construct
  source 1..4204: mol_type = mRNA; note = BNT162b2
  mRNA 1..4204: note = n=(N)1-methylpseudouridine
gagaanaaacnagnanncnncnggnccccacagacncagagagaacccgccaccangnncgngnnccnggngcngcngccncnggngnccagccagngngngaaccngaccaccagaacacagcngccnccagccnacaccaacagcnnnaccagaggcgngnacnaccccgacaaggngnncaganccagcgngcngcacncnacccaggaccngnnccngccnnncnncagcaacgngaccnggnnccacgccanccacgngnccggcaccaanggcaccaagaganncgacaaccccgngcngcccnncaacgacggggngnacnnngccagcaccgagaagnccaacancancagaggcnggancnncggcaccacacnggacagcaagacccagagccngcngancgngaacaacgccaccaacgnggncancaaagngngcgagnnccagnncngcaacgaccccnnccngggcgncnacnaccacaagaacaacaagagcngganggaaagcgagnnccgggngnacagcagcgccaacaacngcaccnncgagnacgngncccagccnnnccnganggaccnggaaggcaagcagggcaacnncaagaaccngcgcgagnncgngnnnaagaacancgacggcnacnncaagancnacagcaagcacaccccnancaaccncgngcgggancngccncagggcnncncngcncnggaaccccnggnggancngcccancggcancaacancacccggnnncagacacngcnggcccngcacagaagcnaccngacaccnggcganagcagcagcgganggacagcnggngccgccgcnnacnangngggcnaccngcagccnagaaccnnccngcngaagnacaacgagaacggcaccancaccgacgccgngganngngcncngganccncngagcgagacaaagngcacccngaagnccnncaccgnggaaaagggcancnaccagaccagcaacnnccgggngcagcccaccgaanccancgngcggnnccccaanancaccaancngngccccnncggcgaggngnncaangccaccaganncgccncngngnacgccnggaaccggaagcggancagcaanngcgnggccgacnacnccgngcngnacaacnccgccagcnncagcaccnncaagngcnacggcgngnccccnaccaagcngaacgaccngngcnncacaaacgngnacgccgacagcnncgnganccggggagangaagngcggcaganngccccnggacagacaggcaagancgccgacnacaacnacaagcngcccgacgacnncaccggcngngnganngccnggaacagcaacaaccnggacnccaaagncggcggcaacnacaannaccngnaccggcngnnccggaagnccaancngaagcccnncgagcgggacancnccaccgagancnancaggccggcagcaccccnngnaacggcgnggaaggcnncaacngcnacnncccacngcagnccnacggcnnncagcccacaaanggcgngggcnancagcccnacagagnggnggngcngagcnncgaacngcngcangccccngccacagngngcggcccnaagaaaagcaccaancncgngaagaacaaangcgngaacnncaacnncaacggccngaccggcaccggcgngcngacagagagcaacaagaagnnccngccannccagcagnnnggccggganancgccganaccacagacgccgnnagaganccccagacacnggaaanccnggacancaccccnngcagcnncggcggagngncngngancaccccnggcaccaacaccagcaancaggnggcagngcngnaccaggacgngaacngnaccgaagngcccgnggccanncacgccgancagcngacaccnacanggcgggngnacnccaccggcagcaangngnnncagaccagagccggcngncngancggagccgagcacgngaacaanagcnacgagngcgacanccccancggcgcnggaancngcgccagcnaccagacacagacaaacagcccncggagagccagaagcgnggccagccagagcancanngccnacacaangncncngggcgccgagaacagcgnggccnacnccaacaacncnancgcnanccccaccaacnncaccancagcgngaccacagaganccngccngngnccangaccaagaccagcgnggacngcaccangnacancngcggcgannccaccgagngcnccaaccngcngcngcagnacggcagcnncngcacccagcngaanagagcccngacagggancgccgnggaacaggacaagaacacccaagaggngnncgcccaagngaagcagancnacaagaccccnccnancaaggacnncggcggcnncaannncagccaganncngcccganccnagcaagcccagcaagcggagcnncancgaggaccngcngnncaacaaagngacacnggccgacgccggcnncancaagcagnanggcganngncngggcgacanngccgccagggancngannngcgcccagaagnnnaacggacngacagngcngccnccncngcngaccgangagangancgcccagnacacancngcccngcnggccggcacaancacaagcggcnggacannnggagcaggcgccgcncngcaganccccnnngcnangcaganggccnaccggnncaacggcancggagngacccagaangngcngnacgagaaccagaagcngancgccaaccagnncaacagcgccancggcaaganccaggacagccngagcagcacagcaagcgcccngggaaagcngcaggacgnggncaaccagaangcccaggcacngaacacccnggncaagcagcngnccnccaacnncggcgccancagcncngngcngaacgananccngagcagacnggacccnccngaggccgaggngcagancgacagacngancacaggcagacngcagagccnccagacanacgngacccagcagcngancagagccgccgagannagagccncngccaancnggccgccaccaagangncngagngngngcngggccagagcaagagagnggacnnnngcggcaagggcnaccaccngangagcnncccncagncngccccncacggcgnggngnnncngcacgngacanangngcccgcncaagagaagaannncaccaccgcnccagccancngccacgacggcaaagcccacnnnccnagagaaggcgngnncgngnccaacggcacccannggnncgngacacagcggaacnncnacgagccccagancancaccaccgacaacaccnncgngncnggcaacngcgacgncgngancggcanngngaacaanaccgngnacgacccncngcagcccgagcnggacagcnncaaagaggaacnggacaagnacnnnaagaaccacacaagccccgacgnggaccngggcganancagcggaancaangccagcgncgngaacanccagaaagagancgaccggcngaacgaggnggccaagaancngaacgagagccngancgaccngcaagaacnggggaagnacgagcagnacancaagnggcccnggnacancnggcngggcnnnancgccggacnganngccancgnganggncacaancangcngngnngcangaccagcngcngnagcngccngaagggcngnngnagcngnggcagcngcngcaagnncgacgaggacganncngagcccgngcngaagggcgngaaacngcacnacacangangacncgagcnggnacngcangcacgcaangcnagcngccccnnncccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacacccnggagcnagcaaaaaaaaaaaaaaaaaaaaaaaaaaaaaa

SEQ ID NO: 7 - RNA, 4204 nt, Synthetic construct
  source 1..4204: mol_type = mRNA; note = Modified BNT162b2
  mRNA 1..4204: note = n=(N)1-methylpseudouridine
gagaanaaacnagnanncnncnggnccccacagacncagagagaacccgccaccangnncgngnnccnggngcngcngccncnggngnccagccagngngngaaccngaccaccagaacacagcngccnccagccnacaccaacagcnnnaccagaggcgngnacnaccccgacaaggngnncaganccagcgngcngcacncnacccaggaccngnnccngccnnncnncagcaacgngaccnggnnccacgccanccacgngnccggcaccaanggcaccaagaganncgacaaccccgngcngcccnncaacgacggggngnacnnngccagcaccgagaagnccaacancancagaggcnggancnncggcaccacacnggacagcaagacccagagccngcngancgngaacaacgccaccaacgnggncancaaagngngcgagnnccagnncngcaacgaccccnnccngggcgncnacnaccacaagaacaacaagagcngganggaaagcgagnnccgggngnacagcagcgccaacaacngcaccnncgagnacgngncccagccnnnccnganggaccnggaaggcaagcagggcaacnncaagaaccngcgcgagnncgngnnnaagaacancgacggcnacnncaagancnacagcaagcacaccccnancaaccncgngcgggancngccncagggcnncncngcncnggaaccccnggnggancngcccancggcancaacancacccggnnccagacacngcnggcccngcacagaagcnaccngacaccnggcganagcagcagcgganggacagcnggngccgccgcnnacnangngggcnaccngcagccnagaaccnnccngcngaagnacaacgagaacggcaccancaccgacgccgngganngngcncngganccncngagcgagacaaagngcacccngaagnccnncaccgnggaaaagggcancnaccagaccagcaacnnccgggngcagcccaccgaanccancgngcggnnccccaanancaccaancngngccccnncggcgaggngnncaangccaccaganncgccncngngnacgccnggaaccggaagcggancagcaanngcgnggccgacnacnccgngcngnacaacnccgccagcnncagcaccnncaagngcnacggcgngnccccnaccaagcngaacgaccngngcnncacaaacgngnacgccgacagcnncgnganccggggagangaagngcggcaganngccccnggacagacaggcaagancgccgacnacaacnacaagcngcccgacgacnncaccggcngngnganngccnggaacagcaacaaccnggacnccaaagncggcggcaacnacaannaccngnaccggcngnnccggaagnccaancngaagcccnncgagcgggacancnccaccgagancnancaggccggcagcaccccnngnaacggcgnggaaggcnncaacngcnacnncccacngcagnccnacggcnnccagcccacaaanggcgngggcnancagcccnacagagnggnggngcngagcnncgaacngcngcangccccngccacagngngcggcccnaagaaaagcaccaancncgngaagaacaaangcgngaacnncaacnncaacggccngaccggcaccggcgngcngacagagagcaacaagaagnnccngccannccagcagnnnggccggganancgccganaccacagacgccgnnagaganccccagacacnggaaanccnggacancaccccnngcagcnncggcggagngncngngancaccccnggcaccaacaccagcaancaggnggcagngcngnaccaggacgngaacngnaccgaagngcccgnggccanncacgccgancagcngacaccnacanggcgggngnacnccaccggcagcaangngnnccagaccagagccggcngncngancggagccgagcacgngaacaanagcnacgagngcgacanccccancggcgcnggaancngcgccagcnaccagacacagacaaacagcccncggagagccagaagcgnggccagccagagcancanngccnacacaangncncngggcgccgagaacagcgnggccnacnccaacaacncnancgcnanccccaccaacnncaccancagcgngaccacagaganccngccngngnccangaccaagaccagcgnggacngcaccangnacancngcggcgannccaccgagngcnccaaccngcngcngcagnacggcagcnncngcacccagcngaanagagcccngacagggancgccgnggaacaggacaagaacacccaagaggngnncgcccaagngaagcagancnacaagaccccnccnancaaggacnncggcggcnncaannncagccaganncngcccganccnagcaagcccagcaagcggagcnncancgaggaccngcngnncaacaaagngacacnggccgacgccggcnncancaagcagnanggcganngncngggcgacanngccgccagggancngannngcgcccagaagnnnaacggacngacagngcngccnccncngcngaccgangagangancgcccagnacacancngcccngcnggccggcacaancacaagcggcnggacannnggagcaggcgccgcncngcaganccccnnngcnangcaganggccnaccggnncaacggcancggagngacccagaangngcngnacgagaaccagaagcngancgccaaccagnncaacagcgccancggcaaganccaggacagccngagcagcacagcaagcgcccngggaaagcngcaggacgnggncaaccagaangcccaggcacngaacacccnggncaagcagcngnccnccaacnncggcgccancagcncngngcngaacgananccngagcagacnggacccnccngaggccgaggngcagancgacagacngancacaggcagacngcagagccnccagacanacgngacccagcagcngancagagccgccgagannagagccncngccaancnggccgccaccaagangncngagngngngcngggccagagcaagagagnggacnncngcggcaagggcnaccaccngangagcnncccncagncngccccncacggcgnggngnnccngcacgngacanangngcccgcncaagagaagaannncaccaccgcnccagccancngccacgacggcaaagcccacnncccnagagaaggcgngnncgngnccaacggcacccannggnncgngacacagcggaacnncnacgagccccagancancaccaccgacaacaccnncgngncnggcaacngcgacgncgngancggcanngngaacaanaccgngnacgacccncngcagcccgagcnggacagcnncaaagaggaacnggacaagnacnnnaagaaccacacaagccccgacgnggaccngggcganancagcggaancaangccagcgncgngaacanccagaaagagancgaccggcngaacgaggnggccaagaancngaacgagagccngancgaccngcaagaacnggggaagnacgagcagnacancaagnggcccnggnacancnggcngggcnnnancgccggacnganngccancgnganggncacaancangcngngnngcangaccagcngcngnagcngccngaagggcngnngnagcngnggcagcngcngcaagnncgacgaggacganncngagcccgngcngaagggcgngaaacngcacnacacangangacncgagcnggnacngcangcacgcaangcnagcngccccnnccccgnccngggnaccccgagncncccccgaccncgggncccaggnangcncccaccnccaccngccccacncaccaccncngcnagnnccagacaccncccaagcacgcagcaangcagcncaaaacgcnnagccnagccacacccccacgggaaacagcagngannaaccnnnagcaanaaacgaaagnnnaacnaagcnanacnaaccccagggnnggncaannncgngccagccacacccnggagcnagcaaaaaaaaaaaaaaaaaaaaaaaaaaaaaa

SEQ ID NO: 8 - DNA, 1723 bp, Synthetic construct
  source 1..1723: mol_type = other DNA; note = T208C
atgggtgactacaaagaccatgacggtgattataaagatcatgacatcgattacaaggatgacgatgacaagctcgaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttcggcaatcaaatcattccggatactgcgattttaagtgttgttccattccatcacggttctggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtg

SEQ ID NO: 9 - DNA, 1723 bp, Synthetic construct
  source 1..1723: mol_type = other DNA; note = AntiFS
atgggtgactacaaagaccatgacggtgattataaagatcatgacatagattacaaggatgacgatgacaagctcgaagacgccaaaaacataaagaaaggcccggcgccattctatccgctagaagatggaaccgctggagagcaactgcataaggctatgaagagatacgccctagttcctggaacaatagcttttacagatgcacatatagaggtagacatcacttacgctgagtacttcgaaatgagcgtaaggttggcagaagctatgaaacgatatgggctgaatacaaatcacagaatagtcgtaagcagtgaaaactctcttcaattctttatgccggtgttaggcgcgttatttatcggagtagcagtagcgcccgcgaacgacatttataatgaacgtgaattgctaaacagtatgggcatttcgcagcctaccgtagtgttcgtagccaaaaaggggttgcaaaaaattttgaacgtgcaaaaaaagctcccaatcatccaaaaaattattataatggattctaaaattttgaacgtgcaaaaaaagctcccaatcatccaaaaaattattataatggattctaaaattaatgaatacgattttgtgccagagtccttcgatagggacaagacaatagcactgataatgaactcctctggatctactggtctgcctaaaggtgtagctctgcctcatagaactgcctgcgtgagattctcgcatgccagagatcctatttttcggcaatcaaatcattccggatactgcgattttaagtgttgttccattccatcacggttttggaatgtttactacactcggatatttgatatgtggataacgagtcgtctaaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgcaggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtg

SEQ ID NO: 10 - protein, 18 aa, Synthetic construct
  source 1..18: note = Spike
MFVFLVLLPLVSSQCVNL

SEQ ID NO: 11 - protein, 16 aa, Synthetic construct
  source 1..16: note = Spike
PLVSSQCVNLTTRTQL

SEQ ID NO: 12 - protein, 18 aa, Synthetic construct
  source 1..18: note = Spike
NLTTRTQLPPAYTNSFTR

SEQ ID NO: 13 - protein, 18 aa, Synthetic construct
  source 1..18: note = Spike
PPAYTNSFTRGVYYPDKV

SEQ ID NO: 14 - protein, 16 aa, Synthetic construct
  source 1..16: note = Spike
TRGVYYPDKVFRSSVL

SEQ ID NO: 15 - protein, 18 aa, Synthetic construct
  source 1..18: note = Spike
KVFRSSVLHSTQDLFLPF

SEQ ID NO: 16 - protein, 17 aa, Synthetic construct
  source 1..17: note = Spike
HSTQDLFLPFFSNVTWF

SEQ ID NO: 17 - protein, 15 aa, Synthetic construct
  source 1..15: note = Spike
LPFFSNVTWFHAIHV

SEQ ID NO: 18 - protein, 15 aa, Synthetic construct
  source 1..15: note = Spike
FHAIHVSGTNGTKRF

SEQ ID NO: 19 - protein, 18 aa, Synthetic construct
  source 1..18: note = Spike
TNGTKRFDNPVLPFNDGV

SEQ ID NO: 20 - protein, 17 aa, Synthetic construct
  source 1..17: note = Spike
NPVLPFNDGVYFASTEK

SEQ ID NO: 21 - protein, 18 aa, Synthetic construct
  source 1..18: note = Spike
GVYFASTEKSNIIRGWIF

SEQ ID NO: 22 - protein, 17 aa, Synthetic construct
  source 1..17: note = Spike
KSNIIRGWIFGTTLDSK

SEQ ID NO: 23 - protein, 16 aa, Synthetic construct
  source 1..16: note = Spike
IFGTTLDSKTQSLLIV

SEQ ID NO: 24 - protein, 18 aa, Synthetic construct
  source 1..18: note = Spike
KTQSLLIVNNATNVVIKV

SEQ ID NO: 25 - protein, 15 aa, Synthetic construct
  source 1..15: note = Spike
NNATNVVIKVCEFQF

SEQ ID NO: 26 - protein, 17 aa, Synthetic construct
  source 1..17: note = Spike
KVCEFQFCNDPFLGVYY

SEQ ID NO: 27 - protein, 17 aa, Synthetic construct
  source 1..17: note = Spike
NDPFLGVYYHKNNKSWM

SEQ ID NO: 28 - protein, 16 aa, Synthetic construct
  source 1..16: note = Spike
YHKNNKSWMESEFRVY

SEQ ID NO: 29 - protein, 18 aa, Synthetic construct
  source 1..18: note = Spike
MESEFRVYSSANNCTFEY

SEQ ID NO: 30 - protein, 17 aa, Synthetic construct
  source 1..17: note = Spike
SSANNCTFEYVSQPFLM

SEQ ID NO: 31 - protein, 18 aa, Synthetic construct
  source 1..18: note = Spike
EYVSQPFLMDLEGKQGNF

SEQ ID NO: 32 - protein, 18 aa, Synthetic construct
  source 1..18: note = Spike
MDLEGKQGNFKNLREFVF

SEQ ID NO: 33 - protein, 18 aa, Synthetic construct
  source 1..18: note = Spike
NFKNLREFVFKNIDGYFK

SEQ ID NO: 34 - protein, 18 aa, Synthetic construct
  source 1..18: note = Spike
VFKNIDGYFKIYSKHTPI

SEQ ID NO: 35 - protein, 16 aa, Synthetic construct
  source 1..16: note = Spike
FKIYSKHTPINLVRDL

SEQ ID NO: 36 - protein, 18 aa, Synthetic construct
  source 1..18: note = Spike
PINLVRDLPQGFSALEPL

SEQ ID NO: 37 - protein, 17 aa, Synthetic construct
  source 1..17: note = Spike
PQGFSALEPLVDLPIGI

SEQ ID NO: 38 - protein, 18 aa, Synthetic construct
  source 1..18: note = Spike
PLVDLPIGINITRFQTLL

SEQ ID NO: 39 - protein, 17 aa, Synthetic construct
  source 1..17: note = Spike
INITRFQTLLALHRSYL

SEQ ID NO: 40 - protein, 18 aa, Synthetic construct
  source 1..18: note = Spike
LLALHRSYLTPGDSSSGW

SEQ ID NO: 41 - protein, 18 aa, Synthetic construct
  source 1..18: note = Spike
LTPGDSSSGWTAGAAAYY

SEQ ID NO: 42 - protein, 17 aa, Synthetic construct
  source 1..17: note = Spike
GWTAGAAAYYVGYLQPR

SEQ ID NO: 43 - protein, 15 aa, Synthetic construct
  source 1..15: note = Spike
YYVGYLQPRTFLLKY

SEQ ID NO: 44 - protein, 17 aa, Synthetic construct
  source 1..17: note = Spike
RTFLLKYNENGTITDAV

SEQ ID NO: 45 - protein, 16 aa, Synthetic construct
  source 1..16: note = Spike
ENGTITDAVDCALDPL

SEQ ID NO: 46 - protein, 18 aa, Synthetic construct
  source 1..18: note = Spike
VDCALDPLSETKCTLKSF

SEQ ID NO: 47 - protein, 17 aa, Synthetic construct
  source 1..17: note = Spike
SETKCTLKSFTVEKGIY

SEQ ID NO: 48 - protein, 16 aa, Synthetic construct
  source 1..16: note = Spike
SFTVEKGIYQTSNFRV

SEQ ID NO: 49 - protein, 17 aa, Synthetic construct
  source 1..17: note = Spike
YQTSNFRVQPTESIVTF

SEQ ID NO: 50 - protein, 18 aa, Synthetic construct
  source 1..18: note = Spike
QPTESIVTFPNITNLCPF

SEQ ID NO: 51 - protein, 18 aa, Synthetic construct
  source 1..18: note = Spike
FPNITNLCPFGEVFNATR

SEQ ID NO: 52 - protein, 17 aa, Synthetic construct
  source 1..17: note = Spike
PFGEVFNATRFASVYAW

SEQ ID NO: 53 - protein, 18 aa, Synthetic construct
  source 1..18: note = Spike
TRFASVYAWNRKRISNCV

SEQ ID NO: 54 - protein, 17 aa, Synthetic construct
  source 1..17: note = Spike
WNRKRISNCVADYSVLY

SEQ ID NO: 55 - protein, 18 aa, Synthetic construct
  source 1..18: note = Spike
CVADYSVLYNSASFSTFK

SEQ ID NO: 56 - protein, 18 aa, Synthetic construct
  source 1..18: note = Spike
YNSASFSTFKCYGVSPTK

SEQ ID NO: 57 - protein, 16 aa, Synthetic construct
  source 1..16: note = Spike
FKCYGVSPTKLNDLCF

SEQ ID NO: 58 - protein, 18 aa, Synthetic construct
  source 1..18: note = Spike
TKLNDLCFTNVYADSFVI

SEQ ID NO: 59 - protein, 18 aa, Synthetic construct
  source 1..18: note = Spike
TNVYADSFVIRGDEVRQI

SEQ ID NO: 60 - protein, 18 aa, Synthetic construct
  source 1..18: note = Spike
VIRGDEVRQIAPGQTGKI

SEQ ID NO: 61 - protein, 17 aa, Synthetic construct
  source 1..17: note = Spike
QIAPGQTGKIADYNYKL

SEQ ID NO: 62 - protein, 18 aa, Synthetic construct
  source 1..18: note = Spike
KIADYNYKLPDDFTGCVI

SEQ ID NO: 63 - protein, 17 aa, Synthetic construct
  source 1..17: note = Spike
LPDDFTGCVIAWNSNNL

SEQ ID NO: 64 - protein, 17 aa, Synthetic construct
  source 1..17: note = Spike
VIAWNSNNLDSKVGGNY

SEQ ID NO: 65 - protein, 18 aa, Synthetic construct
  source 1..18: note = Spike
LDSKVGGNYNYLYRLFRK

SEQ ID NO: 66 - protein, 18 aa, Synthetic construct
  source 1..18: note = Spike
YNYLYRLFRKSNLKPFER

SEQ ID NO: 67 - protein, 17 aa, Synthetic construct
  source 1..17: note = Spike
RKSNLKPFERDISTEIY

SEQ ID NO: 68 - protein, 16 aa, Synthetic construct
  source 1..16: note = Spike
ERDISTEIYQAGSTPC

SEQ ID NO: 69 - protein, 18 aa, Synthetic construct
  source 1..18: note = Spike
YQAGSTPCNGVEGFNCYF

SEQ ID NO: 70 - protein, 17 aa, Synthetic construct
  source 1..17: note = Spike
NGVEGFNCYFPLQSYGF

SEQ ID NO: 71 - protein, 17 aa, Synthetic construct
  source 1..17: note = Spike
YFPLQSYGFQPTNGVGY

SEQ ID NO: 72 - protein, 17 aa, Synthetic construct
  source 1..17: note = Spike
FQPTNGVGYQPYRVVVL

SEQ ID NO: 73 - protein, 15 aa, Synthetic construct
  source 1..15: note = Spike
GYQPYRVVVLSFELL

SEQ ID NO: 74 - protein, 17 aa, Synthetic construct
  source 1..17: note = Spike
LSFELLHAPATVCGPKK

SEQ ID NO: 75 - protein, 17 aa, Synthetic construct
  source 1..17: note = Spike
PATVCGPKKSTNLVKNK

SEQ ID NO: 76 - protein, 18 aa, Synthetic construct
  source 1..18: note = Spike
KSTNLVKNKCVNFNFNGL

SEQ ID NO: 77 - protein, 16 aa, Synthetic construct
  source 1..16: note = Spike
KCVNFNFNGLTGTGVL

SEQ ID NO: 78 - protein, 18 aa, Synthetic construct
  source 1..18: note = Spike
GLTGTGVLTESNKKFLPF

SEQ ID NO: 79 - protein, 17 aa, Synthetic construct
  source 1..17: note = Spike
TESNKKFLPFQQFGRDI

SEQ ID NO: 80 - protein, 17 aa, Synthetic construct
  source 1..17: note = Spike
PFQQFGRDIADTTDAVR

SEQ ID NO: 81 - protein, 17 aa, Synthetic construct
  source 1..17: note = Spike
IADTTDAVRDPQTLEIL

SEQ ID NO: 82 - protein, 16 aa, Synthetic construct
  source 1..16: note = Spike
RDPQTLEILDITPCSF

SEQ ID NO: 83 - protein, 15 aa, Synthetic construct
  source 1..15: note = Spike
ILDITPCSFGGVSVI

SEQ ID NO: 84 - protein, 18 aa, Synthetic construct
  source 1..18: note = Spike
GGVSVITPGTNTSNQVAV

SEQ ID NO: 85 - protein, 15 aa, Synthetic construct
  source 1..15: note = Spike
GTNTSNQVAVLYQDV

SEQ ID NO: 86 - protein, 16 aa, Synthetic construct
  source 1..16: note = Spike
AVLYQDVNCTEVPVAI

SEQ ID NO: 87 - protein, 18 aa, Synthetic construct
  source 1..18: note = Spike
CTEVPVAIHADQLTPTWR

SEQ ID NO: 88 - protein, 18 aa, Synthetic construct
  source 1..18: note = Spike
HADQLTPTWRVYSTGSNV

SEQ ID NO: 89 - protein, 18 aa, Synthetic construct
  source 1..18: note = Spike
WRVYSTGSNVFQTRAGCL

SEQ ID NO: 90 - protein, 16 aa, Synthetic construct
  source 1..16: note = Spike
NVFQTRAGCLIGAEHV

SEQ ID NO: 91 - protein, 18 aa, Synthetic construct
  source 1..18: note = Spike
CLIGAEHVNNSYECDIPI

SEQ ID NO: 92 - protein, 18 aa, Synthetic construct
  source 1..18: note = Spike
NNSYECDIPIGAGICASY

SEQ ID NO: 93 - protein, 18 aa, Synthetic construct
  source 1..18: note = Spike
PIGAGICASYQTQTNSPR

SEQ ID NO: 94 - protein, 15 aa, Synthetic construct
  source 1..15: note = Spike
SYQTQTNSPRRARSV

SEQ ID NO: 95 - protein, 17 aa, Synthetic construct
  source 1..17: note = Spike
PRRARSVASQSIIAYTM

SEQ ID NO: 96 - protein, 17 aa, Synthetic construct
  source 1..17: note = Spike
SQSIIAYTMSLGAENSV

SEQ ID NO: 97 - protein, 18 aa, Synthetic construct
  source 1..18: note = Spike
MSLGAENSVAYSNNSIAI

SEQ ID NO: 98 - protein, 18 aa, Synthetic construct
  source 1..18: note = Spike
VAYSNNSIAIPTNFTISV

SEQ ID NO: 99 - protein, 17 aa, Synthetic construct
  source 1..17: note = Spike
AIPTNFTISVTTEILPV

SEQ ID NO: 100 - protein, 16 aa, Synthetic construct
  source 1..16: note = Spike
SVTTEILPVSMTKTSV

SEQ ID NO: 101 - protein, 15 aa, Synthetic construct
  source 1..15: note = Spike
PVSMTKTSVDCTMYI

SEQ ID NO: 102 - protein, 18 aa, Synthetic construct
  source 1..18: note = Spike
DCTMYICGDSTECSNLLL

SEQ ID NO: 103 - protein, 15 aa, Synthetic construct
  source 1..15: note = Spike
DSTECSNLLLQYGSF

SEQ ID NO: 104 - protein, 18 aa, Synthetic construct
  source 1..18: note = Spike
LLQYGSFCTQLNRALTGI

SEQ ID NO: 105 - protein, 16 aa, Synthetic construct
  source 1..16: note = Spike
TQLNRALTGIAVEQDK

SEQ ID NO: 106 - protein, 18 aa, Synthetic construct
  source 1..18: note = Spike
GIAVEQDKNTQEVFAQVK

SEQ ID NO: 107 - protein, 18 aa, Synthetic construct
  source 1..18: note = Spike
NTQEVFAQVKQIYKTPPI

SEQ ID NO: 108 - protein, 18 aa, Synthetic construct
  source 1..18: note = Spike
VKQIYKTPPIKDFGGFNF

SEQ ID NO: 109 - protein, 15 aa, Synthetic construct
  source 1..15: note = Spike
PPIKDFGGFNFSQIL

SEQ ID NO: 110 - protein, 18 aa, Synthetic construct
  source 1..18: note = Spike
NFSQILPDPSKPSKRSFI

SEQ ID NO: 111 - protein, 18 aa, Synthetic construct
  source 1..18: note = Spike
PSKPSKRSFIEDLLFNKV

SEQ ID NO: 112 - protein, 18 aa, Synthetic construct
  source 1..18: note = Spike
FIEDLLFNKVTLADAGFI

SEQ ID NO: 113 - protein, 17 aa, Synthetic construct
  source 1..17: note = Spike
KVTLADAGFIKQYGDCL

SEQ ID NO: 114 - protein, 18 aa, Synthetic construct
  source 1..18: note = Spike
FIKQYGDCLGDIAARDLI

SEQ ID NO: 115 - protein, 18 aa, Synthetic construct
  source 1..18: note = Spike
LGDIAARDLICAQKFNGL

SEQ ID NO: 116 - protein, 17 aa, Synthetic construct
  source 1..17: note = Spike
LICAQKFNGLTVLPPLL

SEQ ID NO: 117 - protein, 17 aa, Synthetic construct
  source 1..17: note = Spike
GLTVLPPLLTDEMIAQY

SEQ ID NO: 118 - protein, 18 aa, Synthetic construct
  source 1..18: note = Spike
LTDEMIAQYTSALLAGTI

SEQ ID NO: 119 - protein, 16 aa, Synthetic construct
  source 1..16: note = Spike
YTSALLAGTITSGWTF

SEQ ID NO: 120 - protein, 18 aa, Synthetic construct
  source 1..18: note = Spike
TITSGWTFGAGAALQIPF

SEQ ID NO: 121 - protein, 18 aa, Synthetic construct
  source 1..18: note = Spike
GAGAALQIPFAMQMAYRF

SEQ ID NO: 122 - protein, 15 aa, Synthetic construct
  source 1..15: note = Spike
PFAMQMAYRFNGIGV

SEQ ID NO: 123 - protein, 18 aa, Synthetic construct
  source 1..18: note = Spike
RFNGIGVTQNVLYENQKL

SEQ ID NO: 124 - protein, 15 aa, Synthetic construct
  source 1..15: note = Spike
QNVLYENQKLIANQF

SEQ ID NO: 125 - protein, 18 aa, Synthetic construct
  source 1..18: note = Spike
KLIANQFNSAIGKIQDSL

SEQ ID NO: 126 - protein, 17 aa, Synthetic construct
  source 1..17: note = Spike
SAIGKIQDSLSSTASAL

SEQ ID NO: 127 - protein, 16 aa, Synthetic construct
  source 1..16: note = Spike
SLSSTASALGKLQDVV

SEQ ID NO: 128 - protein, 18 aa, Synthetic construct
  source 1..18: note = Spike
LGKLQDVVNQNAQALNTL

SEQ ID NO: 129 - protein, 18 aa, Synthetic construct
  source 1..18: note = Spike
NQNAQALNTLVKQLSSNF

SEQ ID NO: 130 - protein, 17 aa, Synthetic construct
  source 1..17: note = Spike
TLVKQLSSNFGAISSVL

SEQ ID NO: 131 - protein, 16 aa, Synthetic construct
  source 1..16: note = Spike
NFGAISSVLNDILSRL

SEQ ID NO: 132 - protein, 17 aa, Synthetic construct
  source 1..17: note = Spike
LNDILSRLDPPEAEVQI

SEQ ID NO: 133 - protein, 17 aa, Synthetic construct
  source 1..17: note = Spike
DPPEAEVQIDRLITGRL

SEQ ID NO: 134 - protein, 16 aa, Synthetic construct
  source 1..16: note = Spike
IDRLITGRLQSLQTYV

SEQ ID NO: 135 - protein, 18 aa, Synthetic construct
  source 1..18: note = Spike
LQSLQTYVTQQLIRAAEI

SEQ ID NO: 136 - protein, 16 aa, Synthetic construct
  source 1..16: note = Spike
TQQLIRAAEIRASANL

SEQ ID NO: 137 - protein, 18 aa, Synthetic construct
  source 1..18: note = Spike
EIRASANLAATKMSECVL

SEQ ID NO: 138 - protein, 18 aa, Synthetic construct
  source 1..18: note = Spike
AATKMSECVLGQSKRVDF

SEQ ID NO: 139 - protein, 18 aa, Synthetic construct
  source 1..18: note = Spike
VLGQSKRVDFCGKGYHLM

SEQ ID NO: 140 - protein, 15 aa, Synthetic construct
  source 1..15: note = Spike
KRVDFCGKGYHLMSF

SEQ ID NO: 141 - protein, 17 aa, Synthetic construct
  source 1..17: note = Spike
LMSFPQSAPHGVVFLHV

SEQ ID NO: 142 - protein, 17 aa, Synthetic construct
  source 1..17: note = Spike
PHGVVFLHVTYVPAQEK

SEQ ID NO: 143 - protein, 17 aa, Synthetic construct
  source 1..17: note = Spike
VTYVPAQEKNFTTAPAI

SEQ ID NO: 144 - protein, 17 aa, Synthetic construct
  source 1..17: note = Spike
KNFTTAPAICHDGKAHF

SEQ ID NO: 145 - protein, 16 aa, Synthetic construct
  source 1..16: note = Spike
ICHDGKAHFPREGVFV

SEQ ID NO: 146 - protein, 16 aa, Synthetic construct
  source 1..16: note = Spike
FPREGVFVSNGTHWFV

SEQ ID NO: 147 - protein, 18 aa, Synthetic construct
  source 1..18: note = Spike
SNGTHWFVTQRNFYEPQI

SEQ ID NO: 148 - protein, 18 aa, Synthetic construct
  source 1..18: note = Spike
TQRNFYEPQIITTDNTFV

SEQ ID NO: 149 - protein, 18 aa, Synthetic construct
  source 1..18: note = Spike
QIITTDNTFVSGNCDVVI

SEQ ID NO: 150 - protein, 18 aa, Synthetic construct
  source 1..18: note = Spike
FVSGNCDVVIGIVNNTVY

SEQ ID NO: 151 - protein, 17 aa, Synthetic construct
  source 1..17: note = Spike
VIGIVNNTVYDPLQPEL

SEQ ID NO: 152 - protein, 18 aa, Synthetic construct
  source 1..18: note = Spike
VYDPLQPELDSFKEELDK

SEQ ID NO: 153 - protein, 18 aa, Synthetic construct
  source 1..18: note = Spike
LDSFKEELDKYFKNHTSP

SEQ ID NO: 154 - protein, 17 aa, Synthetic construct
  source 1..17: note = Spike
DKYFKNHTSPDVDLGDI

SEQ ID NO: 155 - protein, 17 aa, Synthetic construct
  source 1..17: note = Spike
SPDVDLGDISGINASVV

SEQ ID NO: 156 - protein, 18 aa, Synthetic construct
  source 1..18: note = Spike
ISGINASVVNIQKEIDRL

SEQ ID NO: 157 - protein, 17 aa, Synthetic construct
  source 1..17: note = Spike
VNIQKEIDRLNEVAKNL

SEQ ID NO: 158 - protein, 16 aa, Synthetic construct
  source 1..16: note = Spike
RLNEVAKNLNESLIDL

SEQ ID NO: 159 - protein, 18 aa, Synthetic construct
  source 1..18: note = Spike
LNESLIDLQELGKYEQYI

SEQ ID NO: 160 - protein, 18 aa, Synthetic construct
  source 1..18: note = Spike
QELGKYEQYIKWPWYIWL

SEQ ID NO: 161 - protein, 17 aa, Synthetic construct
  source 1..17: note = Spike
YIKWPWYIWLGFIAGLI

SEQ ID NO: 162 - protein, 18 aa, Synthetic construct
  source 1..18: note = Spike
WLGFIAGLIAIVMVTIML

SEQ ID NO: 163 - protein, 15 aa, Synthetic construct
  source 1..15: note = Spike
GLIAIVMVTIMLCCM

SEQ ID NO: 164 - protein, 15 aa, Synthetic construct
  source 1..15: note = Spike
TIMLCCMTSCCSCLK

SEQ ID NO: 165 - protein, 16 aa, Synthetic construct
  source 1..16: note = Spike
CSCLKGCCSCGSCCKF

SEQ ID NO: 166 - protein, 18 aa, Synthetic construct
  source 1..18: note = Spike
SCGSCCKFDEDDSEPVLK

SEQ ID NO: 167 - protein, 16 aa, Synthetic construct
  source 1..16: note = Spike
DEDDSEPVLKGVKLHY

SEQ ID NO: 168 - protein, 13 aa, Synthetic construct
  source 1..13: note = +1FS spike
YSSGPHRLRENPP

SEQ ID NO: 169 - protein, 15 aa, Synthetic construct
  source 1..15: note = +1FS spike
CSCSWCCCLWCPASV

SEQ ID NO: 170 - protein, 18 aa, Synthetic construct
  source 1..18: note = +1FS spike
PPEHSCLQPTPTALPEAC

SEQ ID NO: 171 - protein, 15 aa, Synthetic construct
  source 1..15: note = +1FS spike
PTPTALPEACTTPTR

SEQ ID NO: 172 - protein, 18 aa, Synthetic construct
  source 1..18: note = +1FS spike
ACTTPTRCSDPACCTLPR

SEQ ID NO: 173 - protein, 15 aa, Synthetic construct
  source 1..15: note = +1FS spike
SDPACCTLPRTCSCL

SEQ ID NO: 174 - protein, 18 aa, Synthetic construct
  source 1..18: note = +1FS spike
PRTCSCLSSATPGSTPST

SEQ ID NO: 175 - protein, 18 aa, Synthetic construct
  source 1..18: note = +1FS spike
SATPGSTPSTCPAPMAPR

SEQ ID NO: 176 - protein, 18 aa, Synthetic construct
  source 1..18: note = +1FS spike
STCPAPMAPRDSTTPCCP

SEQ ID NO: 177 - protein, 17 aa, Synthetic construct
  source 1..17: note = +1FS spike
PRDSTTPCCPSTTGCTL

SEQ ID NO: 178 - protein, 15 aa, Synthetic construct
  source 1..15: note = +1FS spike
PCCPSTTGCTLPAPR

SEQ ID NO: 179 - protein, 17 aa, Synthetic construct
  source 1..17: note = +1FS spike
LPAPRSPTSSEAGSSAP

SEQ ID NO: 180 - protein, 16 aa, Synthetic construct
  source 1..16: note = +1FS spike
SSEAGSSAPHWTARPR

SEQ ID NO: 181 - protein, 15 aa, Synthetic construct
  source 1..15: note = +1FS spike
AGSSAPHWTARPRAC

SEQ ID NO: 182 - protein, 18 aa, Synthetic construct
  source 1..18: note = +1FS spike
TTPPTWSSKCASSSSATT

SEQ ID NO: 183 - protein, 15 aa, Synthetic construct
  source 1..15: note = +1FS spike
SSKCASSSSATTPSW

SEQ ID NO: 184 - protein, 18 aa, Synthetic construct
  source 1..18: note = +1FS spike
TTPSWASTTTRTTRAGWK

SEQ ID NO: 185 - protein, 18 aa, Synthetic construct
  source 1..18: note = +1FS spike
TTRTTRAGWKASSGCTAA

SEQ ID NO: 186 - protein, 18 aa, Synthetic construct
  source 1..18: note = +1FS spike
WKASSGCTAAPTTAPSST

SEQ ID NO: 187 - protein, 15 aa, Synthetic construct
  source 1..15: note = +1FS spike
TAAPTTAPSSTCPSL

SEQ ID NO: 188 - protein, 18 aa, Synthetic construct
  source 1..18: note = +1FS spike
WTWKASRATSRTCASSCL

SEQ ID NO: 189 - protein, 18 aa, Synthetic construct
  source 1..18: note = +1FS spike
TSRTCASSCLRTSTATSR

SEQ ID NO: 190 - protein, 17 aa, Synthetic construct
  source 1..17: note = +1FS spike
CLRTSTATSRSTASTPL

SEQ ID NO: 191 - protein, 18 aa, Synthetic construct
  source 1..18: note = +1FS spike
SRSTASTPLSTSCGICLR

SEQ ID NO: 192 - protein, 18 aa, Synthetic construct
  source 1..18: note = +1FS spike
LSTSCGICLRASLLWNPW

SEQ ID NO: 193 - protein, 15 aa, Synthetic construct
  source 1..15: note = +1FS spike
GICLRASLLWNPWWI

SEQ ID NO: 194 - protein, 18 aa, Synthetic construct
  source 1..18: note = +1FS spike
PWWICPSASTSPGFRHCW

SEQ ID NO: 195 - protein, 15 aa, Synthetic construct
  source 1..15: note = +1FS spike
ICPSASTSPGFRHCW

SEQ ID NO: 196 - protein, 18 aa, Synthetic construct
  source 1..18: note = +1FS spike
HLAIAAADGQLVPPLTMW

SEQ ID NO: 197 - protein, 15 aa, Synthetic construct
  source 1..15: note = +1FS spike
GQLVPPLTMWATCSL

SEQ ID NO: 198 - protein, 15 aa, Synthetic construct
  source 1..15: note = +1FS spike
PPLTMWATCSLEPSC

SEQ ID NO: 199 - protein, 18 aa, Synthetic construct
  source 1..18: note = +1FS spike
STTRTAPSPTPWIVLWIL

SEQ ID NO: 200 - protein, 17 aa, Synthetic construct
  source 1..17: note = +1FS spike
SPSPWKRASTRPATSGC

SEQ ID NO: 201 - protein, 16 aa, Synthetic construct
  source 1..16: note = +1FS spike
STRPATSGCSPPNPSC

SEQ ID NO: 202 - protein, 15 aa, Synthetic construct
  source 1..15: note = +1FS spike
CSPPNPSCGSPISPI

SEQ ID NO: 203 - protein, 16 aa, Synthetic construct
  source 1..16: note = +1FS spike
GSPISPICAPSARCSM

SEQ ID NO: 204 - protein, 15 aa, Synthetic construct
  source 1..15: note = +1FS spike
CAPSARCSMPPDSPL

SEQ ID NO: 205 - protein, 17 aa, Synthetic construct
  source 1..17: note = +1FS spike
PPDSPLCTPGTGSGSAI

SEQ ID NO: 206 - protein, 15 aa, Synthetic construct
  source 1..15: note = +1FS spike
LCTPPGTGSGSAIAW

SEQ ID NO: 207 - protein, 17 aa, Synthetic construct
  source 1..17: note = +1FS spike
IAWPTTPCCTTPPASAP

SEQ ID NO: 208 - protein, 17 aa, Synthetic construct
  source 1..17: note = +1FS spike
CTTPPASAPSSATACPL

SEQ ID NO: 209 - protein, 12 aa, Synthetic construct
  source 1..12: note = +1FS spike
TTCASQTCTPTA

SEQ ID NO: 210 - protein, 16 aa, Synthetic construct
  source 1..16: note = +1FS spike
SGEMKCGRLPLDRQAR

SEQ ID NO: 211 - protein, 18 aa, Synthetic construct
  source 1..18: note = +1FS spike
LPLDRQARSPTTTTSCPT

SEQ ID NO: 212 - protein, 15 aa, Synthetic construct
  source 1..15: note = +1FS spike
SPTTTTSCPTTSPAV

SEQ ID NO: 213 - protein, 17 aa, Synthetic construct
  source 1..17: note = +1FS spike
LPGTATTWTPKSAATTI

SEQ ID NO: 214 - protein, 18 aa, Synthetic construct
  source 1..18: note = +1FS spike
TPKSAATTITCTGCSGSP

SEQ ID NO: 215 - protein, 15 aa, Synthetic construct
  source 1..15: note = +1FS spike
AATTITCTGCSGSPI

SEQ ID NO: 216 - protein, 18 aa, Synthetic construct
  source 1..18: note = +1FS spike
SPSSGTSPPRSIRPAAPL

SEQ ID NO: 217 - protein, 15 aa, Synthetic construct
  source 1..15: note = +1FS spike
PRSIRPAAPLVTAWK

SEQ ID NO: 218 - protein, 18 aa, Synthetic construct
  source 1..18: note = +1FS spike
PLVTAWKASTATSHCSPT

SEQ ID NO: 219 - protein, 18 aa, Synthetic construct
  source 1..18: note = +1FS spike
STATSHCSPTAFSPQMAW

SEQ ID NO: 220 - protein, 18 aa, Synthetic construct
  source 1..18: note = +1FS spike
PTAFSPQMAWAISPTEWW

SEQ ID NO: 221 - protein, 16 aa, Synthetic construct
  source 1..16: note = +1FS spike
ASNCCMPLPQCAALRK

SEQ ID NO: 222 - protein, 15 aa, Synthetic construct
  source 1..15: note = +1FS spike
CMPLPQCAALRKAPI

SEQ ID NO: 223 - protein, 16 aa, Synthetic construct
  source 1..16: note = +1FS spike
QRATRSSCHSSSLAGI

SEQ ID NO: 224 - protein, 18 aa, Synthetic construct
  source 1..18: note = +1FS spike
HSSSLAGISPIPQTPLEI

SEQ ID NO: 225 - protein, 17 aa, Synthetic construct
  source 1..17: note = +1FS spike
SPIPQTPLEIPRHWKSW

SEQ ID NO: 226 - protein, 15 aa, Synthetic construct
  source 1..15: note = +1FS spike
PLEIPRHWKSWTSPL

SEQ ID NO: 227 - protein, 15 aa, Synthetic construct
  source 1..15: note = +1FS spike
WKSWTSPLAASAECL

SEQ ID NO: 228 - protein, 16 aa, Synthetic construct
  source 1..16: note = +1FS spike
SPLAPTPAIRWQCCTR

SEQ ID NO: 229 - protein, 12 aa, Synthetic construct
  source 1..12: note = +1FS spike
TVPKCPWPFTPI

SEQ ID NO: 230 - protein, 15 aa, Synthetic construct
  source 1..15: note = +1FS spike
HLHGGCTPPAAMCFR

SEQ ID NO: 231 - protein, 15 aa, Synthetic construct
  source 1..15: note = +1FS spike
CTPPAAMCFRPEPAV

SEQ ID NO: 232 - protein, 18 aa, Synthetic construct
  source 1..18: note = +1FS spike
TIATSATSPSALESAPAT

SEQ ID NO: 233 - protein, 17 aa, Synthetic construct
  source 1..17: note = +1FS spike
PSALESAPATRHRQTAL

SEQ ID NO: 234 - protein, 18 aa, Synthetic construct
  source 1..18: note = +1FS spike
ATRHRQTALGEPEAWPAR

SEQ ID NO: 235 - protein, 18 aa, Synthetic construct
  source 1..18: note = +1FS spike
LGEPEAWPARASLPTQCL

SEQ ID NO: 236 - protein, 17 aa, Synthetic construct
  source 1..17: note = +1FS spike
ARASLPTQCLWAPRTAW

SEQ ID NO: 237 - protein, 17 aa, Synthetic construct
  source 1..17: note = +1FS spike
CLWAPRTAWPTPTTLSL

SEQ ID NO: 238 - protein, 17 aa, Synthetic construct
  source 1..17: note = +1FS spike
WPTPTTLSLSPPTSPSA

SEQ ID NO: 239 - protein, 8 aa, Synthetic construct
  source 1..8: note = +1FS spike
PQRSCLCP

SEQ ID NO: 240 - protein, 18 aa, Synthetic construct
  source 1..18: note = +1FS spike
PRPAWTAPCTSAAIPPSA

SEQ ID NO: 241 - protein, 18 aa, Synthetic construct
  source 1..18: note = +1FS spike
CTSAAIPPSAPTCCCSTA

SEQ ID NO: 242 - protein, 15 aa, Synthetic construct
  source 1..15: note = +1FS spike
PSAPTCCCSTAASAP

SEQ ID NO: 243 - protein, 17 aa, Synthetic construct
  source 1..17: note = +1FS spike
QGSPWNRTRTPKRCSPK

SEQ ID NO: 244 - protein, 16 aa, Synthetic construct
  source 1..16: note = +1FS spike
SRSTRPLLSRTSAASI

SEQ ID NO: 245 - protein, 16 aa, Synthetic construct
  source 1..16: note = +1FS spike
SRTSAASISARFCPIL

SEQ ID NO: 246 - protein, 18 aa, Synthetic construct
  source 1..18: note = +1FS spike
SARFCPILASPASGASSR

SEQ ID NO: 247 - protein, 16 aa, Synthetic construct
  source 1..16: note = +1FS spike
ASPASGASSRTCCSTK

SEQ ID NO: 248 - protein, 18 aa, Synthetic construct
  source 1..18: note = +1FS spike
HWPTPASSSSMAIVWATL

SEQ ID NO: 249 - protein, 15 aa, Synthetic construct
  source 1..15: note = +1FS spike
SSSMAIVWATLPPGI

SEQ ID NO: 250 - protein, 16 aa, Synthetic construct
  source 1..16: note = +1FS spike
SPSTHLPCWPAQSQAA

SEQ ID NO: 251 - protein, 18 aa, Synthetic construct
  source 1..18: note = +1FS spike
WPAQSQAAGHLEQAPLCR

SEQ ID NO: 252 - protein, 17 aa, Synthetic construct
  source 1..17: note = +1FS spike
GHLEQAPLCRSPLLCRW

SEQ ID NO: 253 - protein, 15 aa, Synthetic construct
  source 1..15: note = +1FS spike
CRSPLLCRWPTGSTA

SEQ ID NO: 254 - protein, 9 aa, Synthetic construct
  source 1..9: note = +1FS spike
PRMCCTRTR

SEQ ID NO: 255 - protein, 13 aa, Synthetic construct
  source 1..13: note = +1FS spike
SPTSSTAPSARSR

SEQ ID NO: 256 - protein, 17 aa, Synthetic construct
  source 1..17: note = +1FS spike
AAQQAPWESCRTWSTRM

SEQ ID NO: 257 - protein, 15 aa, Synthetic construct
  source 1..15: note = +1FS spike
APWESCRTWSTRMPR

SEQ ID NO: 258 - protein, 16 aa, Synthetic construct
  source 1..16: note = +1FS spike
TPWSSSCPPTSAPSAL

SEQ ID NO: 259 - protein, 13 aa, Synthetic construct
  source 1..13: note = +1FS spike
ADWTLLRPRCRST

SEQ ID NO: 260 - protein, 11 aa, Synthetic construct
  source 1..11: note = +1FS spike
SQADCRASRHT

SEQ ID NO: 261 - protein, 18 aa, Synthetic construct
  source 1..18: note = +1FS spike
SEPPRLEPLPIWPPPRCL

SEQ ID NO: 262 - protein, 18 aa, Synthetic construct
  source 1..18: note = +1FS spike
LPIWPPPRCLSVCWARAR

SEQ ID NO: 263 - protein, 17 aa, Synthetic construct
  source 1..17: note = +1FS spike
CLSVCWARAREWTFAAR

SEQ ID NO: 264 - protein, 15 aa, Synthetic construct
  source 1..15: note = +1FS spike
WARAREWTFAARATT

SEQ ID NO: 265 - protein, 14 aa, Synthetic construct
  source 1..14: note = +1FS spike
ASLSLPLTAWCFCT

SEQ ID NO: 266 - protein, 18 aa, Synthetic construct
  source 1..18: note = +1FS spike
HMCPLKRRISPPLQPSAT

SEQ ID NO: 267 - protein, 17 aa, Synthetic construct
  source 1..17: note = +1FS spike
ISPPLQPSATTAKPTFL

SEQ ID NO: 268 - protein, 15 aa, Synthetic construct
  source 1..15: note = +1FS spike
LQPSATTAKPTFLEK

SEQ ID NO: 269 - protein, 15 aa, Synthetic construct
  source 1..15: note = +1FS spike
PTFLEKACSCPTAPI

SEQ ID NO: 270 - protein, 18 aa, Synthetic construct
  source 1..18: note = +1FS spike
HSGTSTSPRSSPPTTPSC

SEQ ID NO: 271 - protein, 15 aa, Synthetic construct
  source 1..15: note = +1FS spike
RSSPPTTPSCLATAT

SEQ ID NO: 272 - protein, 17 aa, Synthetic construct
  source 1..17: note = +1FS spike
TIPCTTLCSPSWTASKR

SEQ ID NO: 273 - protein, 16 aa, Synthetic construct
  source 1..16: note = +1FS spike
SPSWTASKRNWTSTLR

SEQ ID NO: 274 - protein, 17 aa, Synthetic construct
  source 1..17: note = +1FS spike
RNWTSTLRTTQAPTWTW

SEQ ID NO: 275 - protein, 16 aa, Synthetic construct
  source 1..16: note = +1FS spike
TTQAPTWTWAISAESM

SEQ ID NO: 276 - protein, 15 aa, Synthetic construct
  source 1..15: note = +1FS spike
APTWTWAISAESMPA

SEQ ID NO: 277 - protein, 18 aa, Synthetic construct
  source 1..18: note = +1FS spike
STCKNWGSTSSTSSGPGT

SEQ ID NO: 278 - protein, 15 aa, Synthetic construct
  source 1..15: note = +1FS spike
TSSTSSGPGTSGWAL

SEQ ID NO: 279 - protein, 15 aa, Synthetic construct
  source 1..15: note = +1FS spike
STSSGPGTSGWALSP

SEQ ID NO: 280 - protein, 8 aa, Synthetic construct
  source 1..8: note = +1FS spike
WSQSCCVA

SEQ ID NO: 281 - protein, 17 aa, Synthetic construct
  source 1..17: note = +1FS spike
RAVVAVAAAASSTRTIL

SEQ ID NO: 282 - protein, 15 aa, Synthetic construct
  source 1..15: note = +1FS spike
VAAAASSTRTILSPC

SEQ ID NO: 283 - protein, 18 aa, Synthetic construct
  source 1..18: note = +1FS spike
SSWYCMHAMLAAPFPSWV

SEQ ID NO: 284 - protein, 17 aa, Synthetic construct
  source 1..17: note = +1FS spike
MLAAPFPSWVPRVSPDL

SEQ ID NO: 285 - protein, 17 aa, Synthetic construct
  source 1..17: note = +1FS spike
WVPRVSPDLGSQVCSHL

SEQ ID NO: 286 - protein, 18 aa, Synthetic construct
  source 1..18: note = +1FS spike
LGSQVCSHLHLPHSPPLL

SEQ ID NO: 287 - protein, 18 aa, Synthetic construct
  source 1..18: note = +1FS spike
LHLPHSPPLLVPDTSQAR

SEQ ID NO: 288 - protein, 17 aa, Synthetic construct
  source 1..17: note = +1FS spike
LLVPDTSQARSNAAQNA

SEQ ID NO: 289 - protein, 12 aa, Synthetic construct
  source 1..12: note = +1FS spike
PLAINESLTKLY

SEQ ID NO: 290 - protein, 14 aa, Synthetic construct
  source 1..14: note = +1FS spike
PQGWSISCQPHPGA

SEQ ID NO: 291 - protein, 19 aa, Synthetic construct
  source 1..19: note = 9935
GPAPFYPLEDGTAGEQLHK

SEQ ID NO: 292 - protein, 8 aa, Synthetic construct
  source 1..8: note = 33451
YGLNTNHR

SEQ ID NO: 293 - protein, 18 aa, Synthetic construct
  source 1..18: note = 6239
ELLNSMGISQPTVVFVSK

SEQ ID NO: 294 - protein, 7 aa, Synthetic construct
  source 1..7: note = 12509
KLPIIQK

SEQ ID NO: 295 - protein, 7 aa, Synthetic construct
  source 1..7: note = 15467
IIIMDSK

SEQ ID NO: 296 - protein, 16 aa, Synthetic construct
  source 1..16: note = 29537
TIALIMNSSGSTGLPK

SEQ ID NO: 297 - protein, 8 aa, Synthetic construct
  source 1..8: note = 7693
FEEELFLR

SEQ ID NO: 298 - protein, 18 aa, Synthetic construct
  source 1..18: note = 33224
YDLSNLHEIASGGAPLSK

SEQ ID NO: 299 - protein, 8 aa, Synthetic construct
  source 1..8: note = 7351
EVGEAVAK

SEQ ID NO: 300 - protein, 27 aa, Synthetic construct
  source 1..27: note = 23417
QGYGLTETTSAILITPEGDDKPGAVGK

SEQ ID NO: 301 - protein, 8 aa, Synthetic construct
  source 1..8: note = 32478
VVPFFEAK

SEQ ID NO: 302 - protein, 7 aa, Synthetic construct
  source 1..7: note = 29665
TLGVNQR

SEQ ID NO: 303 - protein, 21 aa, Synthetic construct
  source 1..21: note = 9994
GPMIMSGYVNNPEATNALIDK

SEQ ID NO: 304 - protein, 14 aa, Synthetic construct
  source 1..14: note = 6401
EIVDYVASQVTTAK

SEQ ID NO: 305 - protein, 11 aa, Synthetic construct
  source 1..11: note = 9405
GGVVFVDEVPK

SEQ ID NO: 306-311 - skipped (6 entries)

SEQ ID NO: 312 - RNA, 15 nt, synthetic construct
  source 1..15: mol_type = other RNA
cctatttttcggcaa

SEQ ID NO: 313 - RNA, 15 nt, synthetic construct
  source 1..15: mol_type = other RNA
cctattttccggcaa

SEQ ID NO: 314 - RNA, 15 nt, synthetic construct
  source 1..15: mol_type = other RNA
tcacggttttggaat

SEQ ID NO: 315 - RNA, 15 nt, synthetic construct
  source 1..15: mol_type = other RNA
tcacggttctggaat

SEQ ID NO: 316 - RNA, 15 nt, synthetic construct
  source 1..15: mol_type = other RNA
ctgcctaaaggtgtc

SEQ ID NO: 317 - RNA, 15 nt, synthetic construct
  source 1..15: mol_type = other RNA
ctgcctaagggtgtc

SEQ ID NO: 318 - protein, 8 aa, synthetic construct
KIMTSITR

SEQ ID NO: 319 - protein, 7 aa, synthetic construct
KIMTITR

SEQ ID NO: 320 - RNA, 221 nt, synthetic construct
  source 1..221: mol_type = other RNA
  misc_feature 1..221: note = n is (N)1-methylpseudouridine
ncangaacnccncnggancnacnggncngccnaaaggngncgcncngccncanagaacngccngcgngaganncncgcangccagaganccnannnnncggcaancaaancannccgganacngcgannnnaagngnngnnccannccancacggnnnnggaangnnnacnacacncgganannnganangnggannncgagncgncnnaangnanagann

SEQ ID NO: 321 - RNA, 159 nt, synthetic construct
  source 1..159: mol_type = other RNA
  misc_feature 1..159: note = n = (N)1-methylpseudouridine
ngcgngaganncncgcangccagaganccnannnnncggcaancaaancannccgganacngcgannnnaagngnngnnccannccancacggnnnnggaangnnnacnacacncgganannnganangnggannncgagncgncnnaangnanagann

SEQ ID NO: 322 - RNA, 159 nt, synthetic construct; identical to SEQ ID NO: 321
  source 1..159: mol_type = other RNA
  misc_feature 1..159: note = n = (N)1-methylpseudouridine

SEQ ID NO: 323 - RNA, 15 nt, synthetic construct
  source 1..15: mol_type = other RNA
  misc_feature 1..15: note = n=(N)1-methylpseudouridine
acccggnnncagaca

SEQ ID NO: 324 - RNA, 15 nt, synthetic construct
  source 1..15: mol_type = other RNA
  misc_feature 1..15: note = n=(N)1-methylpseudouridine
tacggcnnncagccc

SEQ ID NO: 325 - RNA, 15 nt, synthetic construct
  source 1..15: mol_type = other RNA
  misc_feature 1..15: note = n=(N)1-methylpseudouridine
aatgtgnnncagacc

SEQ ID NO: 326 - RNA, 15 nt, synthetic construct
  source 1..15: mol_type = other RNA
  misc_feature 1..15: note = n=(N)1-methylpseudouridine
gcccacnnncctaga

SEQ ID NO: 327 - RNA, 15 nt, synthetic construct
  source 1..15: mol_type = other RNA
  misc_feature 1..15: note = n=(N)1-methylpseudouridine
gtggacnnnngcggc

SEQ ID NO: 328 - RNA, 15 nt, synthetic construct
  source 1..15: mol_type = other RNA
  misc_feature 1..15: note = n=(N)1-methylpseudouridine
gtggtgnnnctgcac

SEQ ID NO: 329 - RNA, 15 nt, synthetic construct
  source 1..15: mol_type = other RNA
  misc_feature 1..15: note = n=(N)1-methylpseudouridine
tgccccnnncccgtc

SEQ ID NO: 330 - RNA, 25 nt, synthetic construct
  source 1..25: mol_type = other RNA
aaagatcatgacatcgattacaagg

SEQ ID NO: 331 - RNA, 25 nt, synthetic construct
  source 1..25: mol_type = other RNA
aaagatcatgacatagattacaagg

(19) United States
(12) Patent Application Publication
(10) Pub. No.: US 2026/0240987 A1
(43) Pub. Date: Aug. 20, 2026

(54) Therapeutic RNAs

(71) Applicant: CAMBRIDGE ENTERPRISE LIMITED, Cambridge, Cambridgeshire (GB)

(72) Inventors: Anne Elizabeth Willis, Cambridge, Cambridgeshire (GB); Thomas Elliot Mulroney, Cambridge, Cambridgeshire (GB)

(21) Appl. No.: 19/472,380

(22) PCT Filed: Apr. 10, 2024
(86) PCT No.: PCT/GB2024/050941

§ 371 Date: Oct. 3, 2025

(30) Foreign Application Priority Data
Apr. 11, 2023 (GB) 2305297.0

(51) Int. Cl. A61K 39/215; A61K 39/00; A61K 48/00

(52) U.S. Cl. CPC A61K 39/215; A61K 48/005; A61K 2039/53

(57) ABSTRACT

The invention provides modified therapeutic mRNAs that include at least one nucleic acid sequence selected from at least one frameshifting nucleic acid sequence, at least one ribosomal slippery sequence, and/or at least one alternative reading frame sequence wherein the nucleic acid sequence includes at least one synonymous mutation. Also provided are methods of producing said modified therapeutic mRNAs, methods of reducing off-target immunogenicity to a therapeutic mRNA, and methods of reducing out-of-frame translation and/or increasing translation fidelity of a therapeutic mRNA. Further provided are said modified therapeutic mRNAs for use as medicaments.

### FIELD

The present invention provides methods of producing RNAs that have higher translation fidelity and produce lower out-of-frame products when translated. Also provided are RNAs produced by such methods and their use as medicaments.

### BACKGROUND

The efficacy of mRNA therapeutics critically depends on the evasion of the innate immune system and ability to robustly translate a therapeutic protein from exogenously introduced mRNA. Chemical modification of the RNA has historically been used to evade nucleic acid sensors; however, there are conflicting reports as to the levels of protein that ensue from translation of modified mRNAs.

A key feature of therapeutic in vitro transcribed (IVT) mRNAs is that they contain modified ribonucleotides, which have been shown to decrease innate immunogenicity and can additionally increase mRNA stability, both of which are favourable characteristics for therapies ^{1,2}. For example, clinically approved SARS-COV-2 mRNA vaccines incorporate (N)1-methylpseudouridine (1-methylΨ), which has been shown to decrease IVTmRNA innate immunogenicity 3-5. Some modified ribonucleotides, such as 5-methylcytidine (5-methylC), are naturally occurring post-transcriptional mRNA modifications in eukaryotes, while others are not, such as 1-methylΨ⁶⁻¹⁰.

Despite widespread use, surprisingly little is known about how ribonucleotide modification affects protein synthesis, particularly for translation of therapeutic IVTmRNAs. Certain ribonucleotide modifications can recode mRNA sequences, for example, inosine ¹⁵. 5-methylC has previously been shown to increase misreading during mRNA translation in prokaryotes, but its effect on eukaryotic mRNA translation fidelity has not been explored ¹⁶. The effect of 5-methoxyU on translation fidelity has not been investigated. Pseudouridine (Ψ) is known to increase misreading of mRNA stop codons in eukaryotes, and can affect misreading during prokaryotic mRNA translation ¹⁶⁻¹⁸. 1-methylΨ does not appear to affect codon misreading, but has been shown to affect protein synthesis rates and ribosome density on mRNAs, suggesting a direct effect on mRNA translation ^{19,20}.

It is currently unclear which modified ribonucleotides affect mRNA translation fidelity, and existing studies are mostly limited to understanding misreading frequencies at a given codon only. Misreading of mRNA codons is also only one type of posttranscriptional mechanism that can alter a polypeptide sequence. To date, no study has investigated the fundamental question of whether modified ribonucleotides can affect the maintenance of the correct reading frame during translation of a synthetic transcript.

Understanding these processes is critical to increasing knowledge of protein synthesis of modified mRNAs in general but is also imperative for the robust design and evaluation of novel mRNA-based therapeutics that make use of modified ribonucleotides within widely differing RNA sequences or therapeutic contexts.

There is a need for improved mRNA-based therapeutics. There is also a need for improved mRNA-based methods of producing mRNA therapeutics.

### BRIEF SUMMARY OF THE DISCLOSURE

The inventors were interested in how modified ribonucleotides affect the fidelity of mRNA translation. The inventors investigated how 5-methoxyuridine (5-methoxyU), 5-methylC, and 1-methylΨ affect translation of IVTmRNA. 5-methoxyU, 5-methylC, and 1-methylΨ have been utilised in IVTmRNAs to attempt to increase recombinant protein synthesis in vitro, and for preclinical proof-of-concept for IVTmRNA-based therapies ^{11,12}. 1-methylΨ is a ribonucleotide incorporated in licensed IVTmRNA-based SARS-COV2 vaccines, but also mRNA-based human vaccines and therapies in development ^{4,13,14}

The inventors have shown that 1-methylΨ is a modified ribonucleotide that significantly increases +1 ribosomal frameshifting during mRNA translation and that cellular immunity to +1 frameshifted products can occur following vaccination with mRNA containing 1-methylΨ.

This is the first report that mRNA modification affects ribosomal frameshifting. Other ribonucleotide modification strategies, such as the incorporation of 5-methoxyU, significantly decreases translation efficiency of IVTmRNAs, which may limit clinical translation. The inventors have shown that IVTmRNAs contain few nucleotide insertions/deletions, and this is not changed by 1-methylΨ incorporation. +1 ribosomal frameshifting during translation of 1-methylΨ mRNA is linked to ribosome slippery sequences. In addition, translation of mRNA containing 1-methylΨ leads to ribosome stalling. These novel findings are of particular importance to the fundamental understanding of how chemical ribonucleotide modification affects mRNA translation, and for designing and optimising future mRNA-based therapeutics to avoid mistranslation events that may decrease efficacy or increase toxicity.

The inventors have also found that frameshifting events may occur due to alternative reading frames that lead to mistranslation events that may decrease the efficacy or increase the toxicity of mRNA-based therapeutics.

Based on the inventors' findings, the inventors have been able to provide novel methods of producing mRNAs for therapeutic uses that produce lower levels of out-of-frame products by the introduction of synonymous mutations into the mRNA. As such, the inventors have also been able to provide novel mRNAs that include such synonymous mutations and the novel mRNAs for use in various therapeutic methods.

In a first aspect of the invention there is provided a modified therapeutic mRNA comprising at least one frameshifting nucleic acid sequence that increases frequency of out-of-frame translation of the mRNA, wherein the at least one frameshifting nucleic acid sequence comprises at least one synonymous mutation for reducing frequency of out-of-frame translation of the modified therapeutic mRNA.

In a second aspect there is provided a modified therapeutic mRNA comprising at least one ribosomal slippery sequence that increases frequency of out-of-frame translation of the mRNA, wherein the at least one ribosomal slippery sequences comprises at least one synonymous mutation for reducing frequency of out-of-frame translation of the modified therapeutic mRNA.

In a third aspect there is provided a modified therapeutic mRNA comprising at least one at least one alternative reading frame sequence which encodes an alternative translation product that differs from the translation product of in frame translation of the modified therapeutic mRNA and the modified therapeutic mRNA comprising at least one synonymous mutation for introducing a premature termination codon (PTC) in the at least one alternative reading frame sequence.

In a fourth aspect there is provided a nucleic acid encoding a modified therapeutic mRNA of any preceding claim; optionally wherein the nucleic acid comprises a DNA template for in vitro transcription of the modified therapeutic mRNA.

In a fifth aspect there is provided a method of producing a modified therapeutic mRNA having a reduced frequency of out-of-frame translation, the method comprising:
a. providing a therapeutic mRNA or a nucleic acid encoding the therapeutic mRNA;
b. identifying at least one frameshift sequence within the nucleic acid sequence of the therapeutic mRNA or a nucleic acid encoding the therapeutic mRNA;
c. producing a modified therapeutic mRNA comprising a modified at least one frameshift sequence, wherein the modified at least one frameshift sequence comprises at least one synonymous mutation.

In a sixth aspect there is provided a method of reducing off target immunogenicity to a therapeutic mRNA and/or translation product thereof, the method comprising:
a. providing a therapeutic mRNA or a nucleic acid encoding the therapeutic mRNA;
b. identifying at least one frameshift sequence within the nucleic acid sequence of the therapeutic mRNA or a nucleic acid encoding the therapeutic mRNA;
c. producing a modified therapeutic mRNA comprising a modified at least one frameshift sequence, wherein the modified at least one frameshift sequence comprises at least one synonymous mutation; and
d. administering the modified therapeutic mRNA.

In a seventh aspect there is provided a method of reducing out-of-frame translation of a therapeutic mRNA, the method comprising:
a. providing a therapeutic mRNA or a nucleic acid encoding the therapeutic mRNA;
b. identifying at least one frameshift sequence within the nucleic acid sequence of the therapeutic mRNA or a nucleic acid encoding the therapeutic mRNA;
c. producing a modified therapeutic mRNA comprising a modified at least one frameshift sequence, wherein the modified at least one frameshift sequence comprises at least one synonymous mutation; and
d. translating the modified therapeutic mRNA.

In an eighth aspect there is provided a method of increasing translation fidelity of a therapeutic mRNA, the method comprising:
providing a therapeutic mRNA or a nucleic acid encoding the therapeutic mRNA;
identifying at least one frameshift sequence within the nucleic acid sequence of the therapeutic mRNA or a nucleic acid encoding the therapeutic mRNA;
producing a modified therapeutic mRNA comprising a modified at least one frameshift sequence, wherein the modified at least one frameshift sequence comprises at least one synonymous mutation; and
translating the modified therapeutic mRNA to a subject in need thereof.

In certain embodiments, the at least one synonymous mutation increases fidelity of translation of the modified therapeutic mRNA when in use.

In certain embodiments, the out-of-frame translation comprises a +1 frameshift, +2 frameshift, −1 frameshift, or −2 frameshift.

In certain embodiments, the at least one synonymous mutation reduces off target immunogenicity of the modified therapeutic mRNA and/or translation product thereof.

In certain embodiments, the at least one synonymous mutation mutates an out-of-frame codon to a non-cognate amino acid.

In certain embodiments, the off-target immunogenicity comprises cellular immunogenicity.

In certain embodiments, the at least one frameshifting nucleic acid sequence causes ribosome stalling.

In certain embodiments, the at least one frameshifting nucleic acid sequence comprises at least one ribosomal slippery sequence.

In certain embodiments, the at least one ribosomal slippery sequence comprises at least one of the sequences selected from:
a. XXXYYYZ, wherein X is any nucleotide, wherein Y is A or U, and wherein Z is A, U, or C;
b. PPPX, wherein X is any nucleotide and PPP is a trinucleotide repeat of any nucleotide;
c. m1Ψm1Ψm1ΨX, wherein X is any nucleotide and m1Ψ is (N)1-methylpseudouridine;
d. CUUAGG, CUUGAC, CAGCAG, or UCUGCGG; and/or
e. any one of (a) to (d) and sequences or formulae containing non-canonical nucleotides.

In certain embodiments, the PPPX can be decoded by the same isoacceptor tRNA.

In certain embodiments, X is either (N)1-methylpseudouridine or cytidine. For example, in respect of m1Ψ′m1Ψm1ΨX, X may be either (N)1-methylpseudouridine or cytidine.

In certain embodiments, the at least one frameshifting nucleic acid sequence comprises or further comprises at least one alternative reading frame sequence which encodes an out-of-frame product that differs from the translation product of in frame translation of the modified therapeutic mRNA.

In certain embodiments, the at least one synonymous mutation comprises a synonymous mutation for introducing a premature termination codon (PTC) in the one alternative reading frame sequence.

In certain embodiments, the modified therapeutic mRNA comprises at least one chemically modified ribonucleotide.

In certain embodiments, the at least one chemically modified ribonucleotide comprises (N)1-methylpseudouridine.

In certain embodiments, translating comprises translating in vivo or in vitro.

In certain embodiments, in vivo translating comprises administering the modified therapeutic mRNA to a subject in need thereof.

In certain embodiments, identifying comprises sequencing the modified therapeutic mRNA and/or the nucleic acid encoding the modified therapeutic mRNA; and/or analysing the modified therapeutic mRNA sequence and/or the nucleic acid encoding the modified therapeutic mRNA sequence.

In a ninth aspect there is provided modified therapeutic mRNA as described herein for use as a medicament.

In a tenth aspect there is provided modified therapeutic mRNA as described herein for use as a vaccine.

Throughout the description and claims of this specification, the words “comprise” and “contain” and variations of them mean “including but not limited to”, and they are not intended to (and do not) exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith.

Various aspects of the invention are described in further detail below.

### BRIEF DESCRIPTION OF THE FIGURES

Embodiments of the invention are further described hereinafter with reference to the accompanying drawings, in which:

FIG. 1 shows translation of 1-methylΨ-modified mRNA produces +1 frameshifted polypeptides. a, Structures of IVTmRNA transcripts used to probe protein synthesis fidelity. WT Fluc contains (in-frame) Firefly luciferase (Fluc) coding sequence only. Fluc +1FS and Fluc-1FS:Green segments represent in-frame N-terminal Fluc coding sequence, while red or magenta are +1 frameshifted or −1 frameshifted Fluc C-terminal coding sequence, respectively. b, Luciferase activity produced by translation of WTFluc mRNAs, either unmodified control (canonical nucleotides), or containing each of the modified nucleotides indicated. *** ‘P<0.01 (one way ANOVA with Dunnett's test). c, Luciferase activity produced by translation of modified Fluc-1FS mRNAs and unmodified control. d, Luciferase activity produced by translation of modified Fluc +1FS mRNAs and unmodified control. ***’ P<0.01 (one way ANOVA with Dunnett's test) e, Luciferase activity in lysates produced by transfection of Hela cells with unmodified or 1-methylΨ Fluc +1FS mRNA for 8 hours. ‘**’ P<0.01 (Welch's one-tailed T-test). f, Western blot analysis (anti-FLAG epitope) of polypeptides produced by translation of mRNAs in d. All data are obtained from n=3 replicated experiments, d shows a single blot from n=3 replicated experiments

FIG. 2 shows validation of WT Fluc, Fluc +1FS, and Fluc-1FS mRNAs. a, WTFluc, Fluc +1FS, and Fluc-1FS mRNA transcripts. b, Luciferase activity produced by translation of WTFluc, Fluc-1FS, and Fluc +1FS mRNAs.

FIG. 3 shows +1 frameshifted products elicit off-target cellular immune responses following modified mRNA vaccination. a, Depiction of Spike and +1FS products produced by 1-methylΨ-modified Spike mRNA translation. b, Splenocyte IFNγ ELISpot responses from untreated (n=5) and BNT162b2-vaccinated (n=7) mice stimulated with +1FS spike peptides. c, Representative plot of PBMC IFNγ ELISpot response wells for two individuals vaccinated with either BNT162b2 (top) or ChAdOx1 nCOV-19 (bottom). Left to right: In-frame spike response; +1FS spike response; No peptide control. d, Summary data and statistics for PBMC IFNγ ELISpot responses from donors vaccinated with ChAdOx1 nCOV-19 (n=19), or BNT162b2 (n=22), stimulated with +1FS spike peptides. ‘*’ P<0.05, Welch's one-tailed T-test. Undetected responses: BNT162b2 (14/22), ChAdOx1 nCOV-19 (15/19)

FIG. 4 shows mistranslation of 1-methylΨ-mRNA is due to +1 ribosomal frameshifting and not transcriptional errors. a, Peptide coverage plot of the purified high molecular weight polypeptide produced by translation of 1-methylΨ Fluc +1FS mRNA, showing in-frame residues (top) and +1 frameshifted residues (bottom). ‘−10logPEP’ is the mass spectrum percolator score (only high-quality peptides are displayed). b, Nucleotide deletions in unmodified (top) and 1-methylΨ (bottom) Fluc +1FS mRNA, quantified by RNA-seq analysis. c, Nucleotide insertions in unmodified (top) and 1-methylΨ (bottom) Fluc +1FS mRNA.

FIG. 5 correlations between nucleotide insertions or deletions for unmodified Fluc +1FS mRNA and 1-methylΨ′ Fluc +1FS mRNA. Coordinates are (x,y), where x=relative frequency 5′-3′ in unmodified mRNA and y=relative frequency 5′-3′ in 1-methylΨ mRNA of a, nucleotide deletions or b, nucleotide insertions.

FIG. 6 +1 ribosomal frameshifting is dependent on mRNA slippery sequences and associated with ribosome stalling during 1-methylΨ-mRNA translation. a, SDS-PAGE autoradiograph of stalled [³⁵S]-Met-peptidyl-tRNAs produced by translation of unmodified or 1-methylΨ′ Fluc mRNA for 30 minutes. Samples were treated with RNAse to digest peptide-associated tRNAs. Full-length Fluc is indicated by arrow, with intermediate elongating polypeptides below. Stalled peptidyl-tRNAs in 1-methylΨ mRNA translation reactions are indicated by asterisk. b, SDS-PAGE autoradiograph of [³⁵S]-Met-polypeptides produced by translation of unmodified or 1-methylΨ Fluc mRNA for 30 minutes, including or omitting 100 μM paromomycin (+PMN and −PMN, respectively). c, Diagram showing strategy for mRNA slippery sequence mutagenesis. d, +1FS activity after translation of mutant mRNAs, or Fluc +1FS2 control mRNA, for 2 hours. ‘*’ P<0.05 (one way ANOVA with Dunnett's test). e, Total mRNA translation over 2 hours for each of Fluc +1FS mRNA, mutant mRNAs, quantified by [³⁵S]-Met incorporation.

FIG. 7 shows interferon-gamma ELISpot responses to in-frame spike peptide pools from mouse splenocytes and human PBMCs. a, Splenocyte IFNγ ELISpot responses from untreated (n=5) and BNT162b2-vaccinated (n=7) mice stimulated with in-frame SARS-COV2 Spike peptides. b, PBMC IFNγ ELISpot responses from individuals vaccinated with ChAdOx1 nCOV-19 (n=19), or BNT162b2 (n=22), stimulated with in-frame SARS-COV2 Spike peptides. All ChAdOx1 nCOV-19 and n=12 BNT162b2 responses were assayed with total in-frame SARS-COV2 Spike peptides (Spike pool), while n=10 BNT162b2 responses were assayed separately with in-frame SARS-COV2 Spike peptides corresponding to S1/S2 regions (S1+S1 pool). Undetected responses: ChAdOx1 nCOV-19 (4/19), BNT162b2 Spike pool (4/12), BNT162b2 S1+S1 pool (0/10).

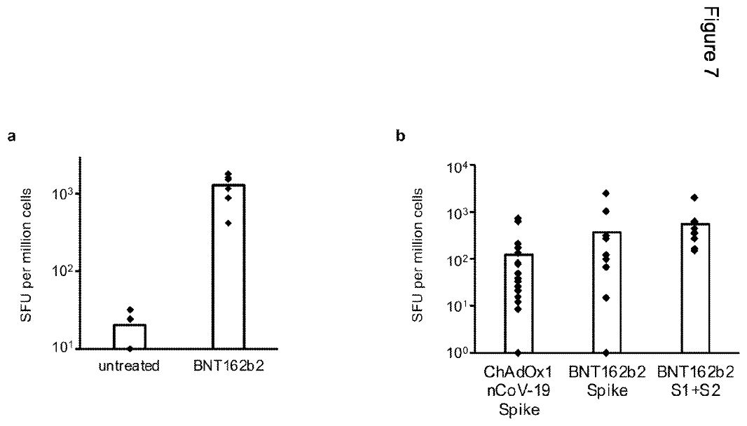

FIG. 8 shows sequence optimisation abrogates ribosome frameshifting. a) Representative example +1PTC introduction in a protein-coding sequence. WT and mutant sequences are synonymous for the encoded peptide KDHDIDYK (SEQ ID NO: 1) in the main frame (0). Synonymous C2A mutation (bold) introduces an amber stop codon in mutant sequence, which encodes a PTC when translated in the +1 frame, and prevents elongation of the undesired +1 peptide. b) Levels of +1 ribosome frameshifting in a firefly luciferase slippery sequence mutant T208C and a synonymous sequence optimised for +1PTCs (AntiFS). Levels are normalised to Fluc +1FS translation.

FIG. 9 shows targeted synonymous mutation of ribosome slippery sites decreases ribosomal +1 frameshifting, while minimally affecting in-frame mRNA translation efficiency. a, Diagram illustrating predicted ribosome slippery sequences and their stop codon-flanked RNA sequence contexts. b, Western blot analysis (anti-FLAG) of polypeptides produced by translation of mRNAs in FIG. 6d and translation of an mRNA containing targeted mutation of Slippery Site B and Slippery Site C (U*187C/U*208C).

FIG. 10 shows ribosomal +1 frameshifting at additional (N)1-methylpseudouridylated ribosome slippery sequences can be decreased by targeted synonymous mutations. a, Depiction of IVT mRNA +1FS reporter. b, Table showing six predicted ribosome slippery sites (Slippery Site 1-6) and one predicted stop codon readthrough ribosome slippery site (SX) and their respective RNA sequence contexts from Sequence ID No. 6. c, Western blot analysis of translation reactions from fourteen mRNAs containing Slippery Sites 1-6 or SX and their respective RNA sequence contexts from Sequence ID No. 6, within the reporter structure depicted in a, either unmodified or containing (N)1-methylΨ. Myc tag expression indicates in-frame mRNA translation, whereas FLAG tag expression indicates +1 frame translation, which is mistranslation indicative of ribosomal +1 frameshifting. d, Targeted synonymous mutation of Slippery Sites 1-6 or SX decreases +1 frame mRNA translation while maintaining in-frame mRNA translation. An anti-Myc tag blot and an anti-FLAG tag blot are displayed from n=2 replicated anti-Myc tag blots and n=2 replicated anti-FLAG tag blots.

The patent, scientific and technical literature referred to herein establish knowledge that was available to those skilled in the art at the time of filing. The entire disclosures of the issued patents, published and pending patent applications, and other publications that are cited herein are hereby incorporated by reference to the same extent as if each was specifically and individually indicated to be incorporated by reference. In the case of any inconsistencies, the present disclosure will prevail.

Various aspects of the invention are described in further detail below.

### DETAILED DESCRIPTION

Therapeutic mRNAs

Provided herein are modified therapeutic mRNAs. The term mRNA refers to an RNA molecule that encodes a protein. mRNA may refer to a ribonucleic acid (RNA) that has been transcribed from a DNA sequence by an RNA polymerase enzyme, and interacts with a ribosome to synthesize protein encoded by DNA. Generally, mRNA is classified into two sub-classes: pre-mRNA and mature mRNA. Precursor mRNA (pre-mRNA) is mRNA that has been transcribed by RNA polymerase but has not undergone any post-transcriptional processing (e.g., 5′capping, splicing, editing, and polyadenylation) and may therefore include 5′ untranslated region (UTR), introns and/or a 3′ UTR (such as a polyadenylation sequence). Mature mRNA has been modified via post-transcriptional processing (e.g., spliced to remove introns and polyadenylated region) and is capable of interacting with ribosomes to perform protein synthesis. The particular nucleic acid sequence composition and length of an mRNA will depend on the protein encoded by the mRNA. Traditionally, the basic components of an mRNA molecule include at least a coding region, a 5′UTR, a 3′UTR, a 5′ cap and a poly-A tail.

In vitro transcribed (IVT) mRNA may function as mRNA but is distinguished from wild-type mRNA in their functional and/or structural design features, which serve to overcome existing problems of effective polypeptide production using nucleic-acid based therapeutics. For example, IVT mRNA may be chemically modified. For example, the polynucleotide “ATCG” may be chemically modified to “AT-5meC-G”.

The therapeutic mRNAs of the invention are referred to as “modified”. In the context of the invention, a modified therapeutic mRNA refers to a therapeutic mRNA that has been modified to introduce a synonymous mutation as described herein. In some cases, the modified therapeutic mRNAs of the invention may further include additional modifications, such as chemically modified nucleotides and/or additional genetic (nucleic acid sequence) modifications.

The therapeutic mRNAs of the invention may comprise naturally occurring ribonucleotides and/or non-naturally occurring ribonucleotides (e.g. canonical nucleotides) such as chemically modified nucleotides. In some examples, the modified therapeutic mRNAs provided herein may include at least one chemically modified ribonucleotide. In some examples, the chemically modified ribonucleotide may be selected from the group consisting of pseudouridine, N1-methylpseudouridine (1-methylΨ), 2-thiouridine, 4′-thiouridine, 5-methylcytosine, 2-thio-I-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, and 2′-O-methyl uridine. Other exemplary chemical modifications useful in mRNAs described herein include those listed in US Published patent application 2015/0064235 which is incorporated herein. In some examples, the modified therapeutic mRNAs provided herein include at least one N1-methylpseudouridine.

The modified therapeutic mRNAs provided herein may be pre-mRNAs or mature mRNAs. In some examples, the modified therapeutic mRNAs provided herein may include one or more features of a pre-mRNA but not all features of a pre-mRNA. For example, the modified therapeutic mRNAs provided herein may include a poly-adenylation sequence but not include introns. In some examples, the modified therapeutic mRNAs provided herein may include one or more features of a mature mRNA but not all features of a mature mRNA “Therapeutic mRNA” refers to an mRNA molecule (e.g., an in vitro transcribed (IVT) mRNA) that encodes a therapeutic protein. Therapeutic proteins mediate a variety of effects in a host cell or a subject in order to treat a disease or ameliorate the signs and symptoms of a disease. For example, a therapeutic protein can replace a protein that is deficient or abnormal, augment the function of an endogenous protein, provide a novel function to a cell (e.g., inhibit or activate an endogenous cellular activity, or act as a delivery agent for another therapeutic compound (e.g., an antibody-drug conjugate). Therapeutic mRNA may be useful for the treatment or prevention of the following diseases and conditions: infectious diseases (such as bacterial infections, viral infections, parasitic infections), cell proliferation disorders (such as cancer), genetic disorders, inflammatory disease, cardiovascular disorders, metabolic diseases, allergic disease, neurodegenerative diseases, protein or enzyme deficiency disorder and/or autoimmune diseases.

Examples of therapeutic mRNAs are Pfizer and BioNtech's BNT162b2 (Covid-19), Moderna's mRNA-1273 (Covid-19), mRNA-2416 (solid tumour or lymphoma), MRT5005 (cystic fibrosis), mRNA-2752 (solid tumour or lymphoma), AZD-8601 (heart failure), NY-ESO-1 (multiple myeloma, synovial sarcoma, melanoma), CTX001 (β-thalassemia), SB-728mR-HSPC (HIV), SB-728mR-T (HIV), BNT163 (HSV2), BNT164 (tuberculosis), BNT165 (malaria), BNT167 (shingles), BNT161 (influenza), BNT153 (undisclosed cancers), BNT152 (undisclosed cancers), BNT142 (undisclosed cancers), BNT141 (undisclosed cancers), BNT131 (undisclosed cancers), BNT122 (melanoma), colorectal cancer, BNT116 (non-small cell lung carcinoma), BNT115 (ovarian cancer), BNT113 (head and neck cancer), BNT112 (prostate cancer), BNT111 (melanoma), mRNA-1345 (Respiratory syncytial virus), mRNA-1010 (influenza), mRNA-1647 (cytomegalovirus), mRNA-4157/V940 (melanoma), mRNA-3927 (Propionic acidemia), mRNA-0184 (heart failure), VX-522 (cystic fibrosis). The uses of each mRNA therapy are shown in brackets.

Therapeutic mRNA molecules are generally synthesized in a laboratory (e.g., by in vitro transcription). mRNA can be isolated from tissues or cells by a variety of methods. For example, a total RNA extraction can be performed on cells or a cell lysate, and the resulting extracted total RNA can be purified (e.g., on a column comprising oligo-dT beads) to obtain extracted mRNA. Alternatively, mRNA can be synthesized in a cell-free environment, for example, by in vitro transcription (IVT). IVT is a process that permits template-directed (e.g. via an IVT DNA template) synthesis of a ribonucleic acid (RNA) (e.g., messenger RNA (mRNA)). It is based, generally, on the engineering of a DNA template that includes a bacteriophage promoter sequence upstream of the sequence of interest, followed by transcription using a corresponding RNA polymerase. In vitro mRNA transcripts, for example, may be used as therapeutics in vivo to direct ribosomes to express protein therapeutics within targeted tissues.

An “in vitro transcription template (IVT),” as used herein, refers to deoxyribonucleic acid (DNA) suitable for use in an IVT reaction for the production of messenger RNA (mRNA). In some examples, an IVT template encodes a 5′ untranslated region, contains an open reading frame, and encodes a 3′ untranslated region and a polyA tail. The particular nucleotide sequence composition and length of an IVT template will depend on the mRNA of interest encoded by the template. IVT mature mRNA preparation includes several steps, linear DNA template obtainment, IVT, 5′ capping, and poly(A) tail adding.

A “5′ untranslated region (UTR)” refers to a region of an mRNA that is directly upstream (i.e., 5′) from the start codon (i.e., the first codon of an mRNA transcript translated by a ribosome) that does not encode a protein or peptide. IVT mRNA is performed with linearizing plasmid DNA templates or PCR templates requiring at least a promoter and the corresponding mRNA construct sequence. IVT mRNA may be carried out by adding polymerases (T7, T3, or SP6) but requires additional capping. Uncapped mRNA is rapidly degraded by RNase and contains a 5′-ppp group, which causes greater immune stimulation and can be treated with phosphatase to reduce undesirable efficacy. Two methods may be implemented for the capping of IVT mRNA: co-transcriptional capping and posttranscriptional capping. Cap dinucleotide mixtures containing four nucleoside triphosphates (NTPs) are incorporated at the 5′ end of the RNA with RNA polymerase during co-transcriptional capping. Co-transcriptional capping processing has permitted coordinated transcription with mRNA capping. Poly (A) tails of IVT mRNAs are normally encoded in the DNA template or attached to IVT mRNA by enzymatic polyadenylation. The former may have more precise control of the length of the poly(A) tail. IVT mRNAs are mixed with RNA polymerase and DNA templates after synthesis; thus, purification of IVT mRNA may be needed, including removing immunostimulatory contaminants, free ribonucleotides, short mRNA and DNA templates. Generally, Dnase is used to degrade excess DNA templates. Commercial purification kits may be used to purify and separate the synthesized mRNA, followed by precipitation using ethanol or isopropanol, which can remove most contaminants and obtain high-purity mRNA, and then the mRNA may be precipitated with high concentrations of LiCl or alcohol-based precipitation, chromatographic methods (molecular exclusion chromatography, ion-exchange chromatography, or affinity chromatography with immobilized oligo-dT), or elution from a silica membrane column, which removes proteins, free nucleotides or other components but not dsRNA impurities. To remove dsRNA contaminants from the transcription reaction solution, reversed-phase HPLC may be used.

A “3′ untranslated region (UTR)” refers to a region of an mRNA that is directly downstream (i.e., 3′) from the stop codon (i.e., the codon of an mRNA transcript that signals a termination of translation) that does not encode a protein or peptide.

A “polyA tail” is a region of mRNA that is downstream, e.g., directly downstream (i.e., 3′), from the 3′ UTR that contains multiple, consecutive adenosine monophosphates. A polyA tail may contain 10 to 300 adenosine monophosphates. For example, a polyA tail may contain 10, 20, 30, 40, 50, 60, 70, 80, 90, 100, 110, 120, 130, 140, 150, 160, 170, 180, 190, 200, 210, 220, 230, 240, 250, 260, 270, 280, 290 or 300 adenosine monophosphates. In some examples, a polyA tail contains 50 to 250 adenosine monophosphates. In a relevant biological setting (e.g., in cells, in vivo, etc.), the poly(A) sitail functions to protect mRNA from enzymatic degradation, e.g., in the cytoplasm, and aids in transcription termination, export of the mRNA from the nucleus, and translation. However, in some examples, mRNA molecules provided herein do not comprise a polyA tail (such molecules are referred to as “tailless”). An “open reading frame” is a continuous stretch of DNA or RNA beginning with a start codon (e.g., methionine (ATG)) and ending with a stop codon (e.g., TAA, TAG or TGA) and encodes a protein or peptide.

### Frameshifting Nucleic Acid Sequence

The modified therapeutic mRNAs provided herein may include at least one frameshifting nucleic acid sequence. The term “frameshifting nucleic acid sequence” is used to refer to any nucleic acid sequences, such as an RNA sequence or DNA sequence of a template for IVT encoding modified therapeutic mRNA of the invention, that may cause or increase the likelihood of frameshifting of a ribosome (i.e. translation or ribosomal frameshifting). “Frameshift” refers to a process in which a ribosome shifts to an alternative reading frame (altered frame) by one or a few nucleotides at a site in an mRNA. Frameshifts may result in the production of multiple, unique proteins from a single mRNA. Proteins are translated by reading tri-nucleotides (codons) from the 5′ to the 3′ end, starting with the amino acid methionine as the start (initiation) codon. Each codon is translated into a single amino acid. The code itself is degenerate, meaning that a particular amino acid can be specified by more than one codon. A shift of any number of nucleotides that is not divisible by 3 in the reading frame will cause subsequent codons to be read differently from that of the intended or correct reading frame. This effectively changes the ribosomal reading frame leading to the production of alternative polypeptides encoded by mRNA. The translation of a frameshifted codon may be referred to as out-of-frame translation, and the products (i.e. alternative proteins or polypeptides) may be referred to as out-of-frame products or proteins or as alternative products or proteins (i.e. alternative to the product encoded by the non-frameshifted (correct or in frame translation product) open reading frame). Frameshifting nucleic acid sequences lead to alternative translation products such as truncated proteins (e.g. due to premature termination of translation), proteins having an alternative or different amino acid sequence to that encoded the non-frameshifted open reading frame or an increased amino acid length from the desired product produced by translation in the correct open reading frame (e.g. due to misreading or read-through of stop codons).

Frameshifting nucleic acid sequences may be any sequences that are known or predicted to lead to ribosomal frameshifting. Methods to identify or predict frameshifting nucleic acid sequences are known. For example, US20080103745A1 describes a model for predicting frameshifts. Other methods for predicting ribosomal frameshifts include those described in Moon, S. et al., LNCS, 2004, 3036:334-34; Hammell, A. B. et al., Genomic Res., 1999, 9:417-427); Bekaert, M. et al., Bioinformatics, 2003, 19:327-335) and Shah, A. A. et al., Bioinformatics, 2002, 18:1046-1053.

Each frameshift may be a −2, −1, +1 or +2 frameshift. “−1 frameshift” refers to a frameshift in which a ribosome shifts a nucleotide in the upstream direction, and “+1 frameshift” refers to a frameshift in which a ribosome shifts a nucleotide in the downstream direction.

Known frameshifting nucleic acid sequences that may cause a +1 frameshift include: sequences of UUUUGA (SEQ ID NO:2), UCCUGA (SEQ ID NO:3) or CCCUGA (SEQ ID NO: 4); spacer components having a spacer with 4 to 11 nucleotides; and/or secondary structures capable of designating stem-loops or pseudoknots.

Known frameshifting nucleic acid sequences that may cause a-1 or +1 frameshift include sites that comprise sequentially a sequence of XXXYYYZ (SEQ ID NO: 5), wherein X is any nucleotide, wherein Y is A or U, and wherein Z is A, U, or C; space components with 4 to 11 nucleotides; and/or secondary structures component capable of designating stem-loops or pseudoknots.

In some examples, the frameshifting nucleic acid sequences may comprise a sequence of PPPX (SEQ ID NO: 306), wherein X is any nucleotide, and PPP is a trinucleotide repeat of any nucleotide. In some examples, the frameshifting nucleic acid sequences may comprise a sequence of m1Ψm1Ψm1ΨX (SEQ ID NO: 307), wherein X is any nucleotide and m1Ψ′ is (N)1-methylpseudouridine. In some examples, the frameshifting nucleic acid sequences may comprise a sequence of CUUAGG (SEQ ID NO: 308), CUUGAC (SEQ ID NO: 309), CAGCAG (SEQ ID NO: 310), or UCUGCGG (SEQ ID NO: 311).

In some examples, the frameshifting nucleic acid sequences may comprise a sequence that includes non-canonical amino acids. Non-canonical amino acids are non-proteinogenic amino acids that are either found naturally in organisms or are synthetically made in a laboratory that are not located in the genetic code of naturally occurring organisms.

In some examples, the frameshifting nucleic acid sequences may comprise a combination of any of the sequences described above.

The frameshifting nucleic acid sequences may, in some cases, be described as ribosomal slippery sequences. As such, in some examples, the modified therapeutic mRNAs of the invention may comprise one or more ribosomal slippery sequences.

Pseudoknots are secondary RNA substructures that contain two or more stem-loop motifs with intercalated stems. The pseudoknot or stem-loop structure in the mRNA is thought to result in pausing of the ribosome, resulting in eventual frameshifting.

In some examples, frameshifting nucleic acid sequences may also cause ribosomal stalling. In some examples, frameshifting nucleic acid sequences are any sequence that may cause ribosomal stalling. Sequences that cause ribosomal stalling are known and may include sequences that encode mRNA secondary structure, runs of rare or difficult-to-decode codons, and codons encoding certain amino acids such as proline, glycine, positively charged amino acids, and negatively charged amino acids.

Other frameshifting nucleic acid sequences will be known. For example, the frameshifting nucleic acid sequence may be sequence as identified in a database such as the FSDB (see Moon S, Byun Y, Han K. FSDB: a frameshift signal database. Comput Biol Chem. 2007; 31 (4): 298-302. Doi: 10.1016/j.compbiolchem.2007.05.004) or the PRFdb (see Belew, Ashton T., et al. “PRFdb: a database of computationally predicted eukaryotic programmed-1 ribosomal frameshift signals.” BMC genomics 9.1 (2008): 1-7.).

In some examples, the frameshifting nucleic acid sequence is an alternative reading frame sequence. For example, the frameshifting nucleic acid sequence may be a sequence that encodes a different reading frame in the −2, −1, +1, or +2 position from the correct open reading frame that leads to the production of an alternative or out-of-frame protein. Alternative reading frame sequences may be sequences that cause ribosomal stalling or may be located in close proximity to sequences that cause ribosomal stalling (e.g. at a position from about-2 to about +2 nucleotides from the sequences that cause ribosomal stalling).

In some examples, the frameshifting nucleic acid sequence may be considered a slippery sequence, encode an alternative reading frame sequence and/or cause ribosomal stalling. In some examples, the frameshifting nucleic acid sequence comprises a modified ribonucleotide, such as chemically modified ribonucleotides, as described herein. In some examples, the frameshifting nucleic acid sequence includes (N)1-methylpseudouridine.

Throughout the description, reference is made to analysing the sequence of the therapeutic mRNA. It will be understood that analysis and/or sequencing may be performed on an mRNA, an RNA sequence or on a template for production of an mRNA or RNA sequence. For example, analysis of a DNA template used for production of an mRNA (for example, by IVT) may be carried out to identify DNA sequences that encode for frameshifting nucleic acid sequences as described herein after transcription.

Therefore, also provided herein are nucleic acids encoding a modified therapeutic mRNA as described herein. In some examples, there is provided a DNA template for in vitro transcription of a modified therapeutic mRNA as described herein.

The out-of-frame and/or alternative translation products may be more immunogenic in comparison to the in-frame translation product. The out-of-frame and/or alternative translation products may also have a reduced efficacy in comparison to the in-frame translation product. Frameshifting nucleic acid sequence as described herein may lead to a decreased efficiency and/or fidelity (accuracy to the intended translation product).

### Synonymous Mutations

Without being bound by theory, the inventors have found that the introduction of one or more synonymous mutations at frameshifting nucleic acid sequences may help reduce out-of-frame translation and, ergo, the production of out-of-frame or alternative translation products.

“Synonymous mutation” refers to a change, relative to a reference sequence, in an mRNA as described herein (e.g., at 1, 2, 3, 4, 5, 6, 7, 8, 9, 10, or more nucleotides relative to the reference sequence), wherein the change does not alter the amino acid that is encoded. For example, GGT, GGA, GGC, and GGG all code for glycine. Any change in the third position of the codon (e.g. A->G) will result in the same amino acid being incorporated into the protein sequence at that position.

The introduction of a synonymous mutation may therefore lead to a decrease in a level of out-of-frame translation products produced upon translation of a modified therapeutic mRNA as described herein in comparison to a therapeutic mRNA that does not include the synonymous mutation. As such, the synonymous mutation may lead to an increase in fidelity of translation in comparison to a therapeutic mRNA that does not include the synonymous mutation.

“Fidelity of translation” refers to the accuracy of translation of an mRNA. For example, the translation of the desired protein (comprising the amino acids encoded by the in-frame codons) which is produced by translation occurring in the intended reading frame (i.e. correct or in-frame translation). In some examples, the synonymous mutation may increase the efficiency of translation. For example, the synonymous mutation may reduce or prevent ribosomal stalling, which, therefore, may allow for increased rates of translation in comparison to a therapeutic mRNA that does not include the synonymous mutation.

In some examples, the synonymous mutation may introduce a premature termination codon. “Premature termination codon” or “premature stop codon” refers to a stop codon in an mRNA (prior to the endogenous or desired termination codon) as the result of a mutation (i.e. nucleic acid modification). Premature termination codon (PTC) may include one of three stop codons: UAA; UAG; or UGA. The modified therapeutic mRNAs provided herein may comprise a premature stop codon which is encoded in a frameshifted reading frame. For example, when translated in-frame, the translation product is unaffected by the synonymous mutation and inserted PTC is not read as a PTC. However, if frameshifting occurs (i.e. a-1, −2, +1, or +2 frameshift), the frameshifted reading frame comprises at least one codon that encodes a PTC that results in termination of translation and production of a truncated protein that may be degraded when produced in a subject. Such synonymous mutations may prevent out-of-frame translation as well as helping destabilise mRNAs undergoing out-of-frame translation.

The synonymous mutation may introduce an out-of-frame codon (i.e. frameshifted codon) that encodes for a non-cognate amino acid. Aa-tRNAs that can participate in standard Watson-Crick interactions with the first two bases in a codon and can form either canonical or non-Watson-Crick pairs at the third or “wobble” position are designated cognate-tRNAs. In contrast, tRNAs that do not meet these requirements are commonly referred to as near-and non-cognate tRNAs.

As the synonymous mutation reduces out-of-frame translation and the products thereof, the translation products of a modified therapeutic mRNA, including the synonymous mutation, may have decreased immunogenicity in comparison to the translation products of an mRNA not including the synonymous mutation. In some examples, the translation products may have decreased innate immunogenicity. In some examples, the translation products may have decreased cellular immunogenicity.

Also provided herein are nucleic acids encoding a modified therapeutic mRNA as described, including a synonymous mutation as described herein. In some examples, there is provided a DNA template for in vitro transcription of a modified therapeutic mRNA as described herein, including a synonymous mutation as described.

The modifications and methods of producing modified therapeutic mRNAs may be applied to any known therapeutic mRNA. For example, one of the therapeutic mRNAs described above (BNT162b2, Moderna's mRNA-1273, mRNA-2416, MRT5005, AZD-8601, NY-ESO-1, CTX001, SB-728mR-HSPC, and SB-728mR-T). For example, there is provided a therapeutic mRNA according to SEQ ID NO: 6 modified to include at least one synonymous mutation as described herein. In one example, there is provided a modified therapeutic mRNA according to SEQ ID NO: 7 or a DNA template for IVT encoding a modified therapeutic mRNA according to SEQ ID NO: 7.

### Medical Uses

The modified therapeutic mRNAs may be used for a number of in vitro and in vivo uses. Reference to medicals uses, modified therapeutic mRNAs for use in methods of treatment and methods of manufacturing a medicament using the modified therapeutic mRNAs are all to be understood to relate to methods of treating a subject using the modified therapeutic mRNAs as described herein. Thus in one example, there is provided a modified therapeutic mRNA as described herein for manufacture of a medicament for treating any of the disorders, conditions or diseases described herein. In one example, there is provided a modified therapeutic mRNA as described herein for use in treating any of the disorders, conditions or diseases described herein. In one example, there is provided a modified therapeutic mRNA as described herein for treating any of the disorders, conditions or diseases described herein. In one example, there is provided a method of treating a subject in need thereof comprising administering a modified therapeutic mRNA as described herein. For example, the method is a method for treating any of the disorders, conditions or diseases described herein.

The modified therapeutic mRNAs described herein may be for use as medicaments. For example, the modified therapeutic mRNAs described herein may be for use in methods of preventing or treating a disease or condition in a subject. The disease or condition treated may depend on the protein encoded by the modified therapeutic mRNA. In general, the modified therapeutic mRNA described herein may be for use in treating any diseases or conditions that may benefit from the administration of an mRNA or protein translated therefrom. The modified therapeutic mRNAs may be for use or used in methods of treating infectious diseases (such as bacterial infections, viral infections, parasitic infections), cell proliferation disorders (such as cancer), genetic disorders, inflammatory disease, cardiovascular disorders, metabolic diseases, allergic disease, neurodegenerative diseases, protein or enzyme deficiency disorder and/or autoimmune diseases.

For example, the modified therapeutic mRNAs described herein may be for use in methods of treating a genetic disorder. “Genetic disorder” refers to a congenital or acquired disease caused by chromosomal or mitochondrial DNA abnormalities, and examples thereof include Down syndrome, Wilson disease, Edwards syndrome, Patau syndrome, Turner syndrome, Klinefelter syndrome, Apert syndrome, Crouzon syndrome, 22q11.2 deletion syndrome, Williams syndrome, Laurence-Moon-Biedl syndrome, Prader-Willi syndrome, Angelman syndrome, Kallmann syndrome, Aicardi-Goutieres syndrome, Miller-Dieker syndrome, Rubinstein-Taybi syndrome, Cornelia de Lange syndrome, cri-du-chat syndrome, super female, super male, and mitochondrial disease.

For example, the modified therapeutic mRNAs described herein may be for use in methods of treating a protein or enzyme deficiency disorder. “Protein or enzyme deficiency disorders” refers to any disease or disorder that is associated with a subject lacking one or more proteins or enzymes or lacking sufficient activity of a protein or enzyme leading to symptoms and adverse effects in the subject. Examples of protein or enzyme deficiency disorders include Pompe disease, mucopolysaccharidosis types I, II, and VI, hemophilias A and B hypercystinia, Danon's disease, myoclonic renal failure syndrome, sialic acid storage disorders such as ISSD, Salla disease and moderately severe Salla disease, Niemann-Pick disease C1 and C2, mucolipid accumulation disease type IV; neuronal ceroid lipofuscinosis includes but not limited to ceroid lipofuscinosis 1 Types (Haltia-Santavuori disease and INCL), neuronal ceroid lipofuscinosis type 2 (Jansky-Bielschowsky disease), cereoid lipofuscinosis type 3 (Batten-Spielmeyer-Sjogren disease), waxy Lipofuscinosis type 4 (Parry's disease and Kufs A and B), cereoid lipofuscinosis type 5 (late infant Finnish type), cereofuscinosis type 6 (Lake-Cavanagh) Or Indian type), cereoid lipofuscinosis type 7 (Turkish type), cereoid lipofuscinosis type 8 (Northern epilepsy, epilepsy mental disorder), cereoid lipofuscinosis 9, cereoid lipofuscinosis 10, cereoid lipofuscinosis 11, cereoid lipofuscinosis 12, cereoid Lipofuscinosis 13, Cereofuscinosis 14; Lysosomal-related organelle disorders including but not limited to Hermansky-Pudlak disease type 1, Hermansky-Pudlak disease type 2, Hermansky-Pudlak disease type 3, Hermansky-Pudlak disease type 4, Hermansky-Pudlak disease type 5, Hermansky-Pudlak disease type 6, Hermansky-Pudlak disease type 7, Hermansky-Pudlak disease type 8, Hermansky-Pudlak disease type 9, Griscelli syndrome 1 (Elejalde syndrome)), Griscelli syndrome 2, Chédiak-Higashi disease, lysosomal storage disorders, e.g. Hurler syndrome, Niemann-Pick disease, Tay-Sachs disease, Gaucher disease, Fabry disease or Krabbe disease; Phenylketonuria; mitochondrial disorders; Friedreich ataxia; peroxisomal disorders, e.g. Zellweger syndrome or Adrenoleukodystrophy; metal metabolism disorders, e.g. Wilson disease or hemochromatosis; organic acidemias, e.g. methylmalonic acidemia or propionic acidemia; urea cycle disorders, e.g. ornithine transcarbamylase deficiency or citrullinemia and/or β-thalassemia. Other examples of enzyme deficiency disorders include Type I diabetes mellitus, which results from the patient's failure to produce insulin.

For example, the modified therapeutic mRNAs described herein may be for use in methods of treating cardiovascular disorders. “Cardiovascular disease” or “cardiovascular disorder” refers to diseases affecting the heart or blood vessels or both. For example, cardiovascular disease includes arrhythmia (atrial or ventricular or both); atherosclerosis and its sequelae; angina; cardiac rhythm disturbances; myocardial ischemia; myocardial infarction; cardiac or vascular aneurysm; vasculitis, stroke; peripheral obstructive arteriopathy of a limb, an organ, or a tissue; reperfusion injury following ischemia of the brain, heart, kidney or other organ or tissue; endotoxic, surgical, or traumatic shock; hypertension, valvular heart disease, heart failure, abnormal blood pressure; shock; vasoconstriction (including that associated with migraines); vascular abnormality, insufficiency limited to a single organ or tissue.

For example, the modified therapeutic mRNAs described herein may be for use in methods of treating an autoimmune disease. “Autoimmune disease” refers to a disease or condition in which a subject's immune system has an aberrant immune response against a substance that does not normally elicit an immune response in a healthy subject. Examples of autoimmune diseases that may be treated include Acute Disseminated Encephalomyelitis (ADEM), Acute necrotizing hemorrhagic leukoencephalitis, Addison's disease, Agammaglobulinemia, Alopecia areata, Amyloidosis, Ankylosing spondylitis, Anti-GBM/Anti-TBM nephritis, Antiphospholipid syndrome (APS), Autoimmune angioedema, Autoimmune aplastic anemia,

Autoimmune dysautonomia, Autoimmune hepatitis, Autoimmune hyperlipidemia, Autoimmune immunodeficiency, Autoimmune inner ear disease (AIED), Autoimmune myocarditis, Autoimmune oophoritis, Autoimmune pancreatitis, Autoimmune retinopathy, Autoimmune thrombocytopenia purpura (ATP), Autoimmune thyroid disease, Autoimmune urticaria, Axonal or neuronal neuropathies, Balo disease, Behcet's disease, Bullous pemphigoid, Cardiomyopathy, Castleman disease, Celiac disease, Chagas disease, Chronic fatigue syndrome, Chronic inflammatory demyelinating polyneuropathy (CIDP), Chronic recurrent multifocal ostomyelitis (CRMO), Churg-Strauss syndrome, Cicatricial pemphigoid/benign mucosal pemphigoid, Crohn's disease, Cogans syndrome, Cold agglutinin disease, Congenital heart block, Coxsackie myocarditis, CREST disease, Essential mixed cryoglobulinemia, Demyelinating neuropathies, Dermatitis herpetiformis, Dermatomyositis, Devic's disease (neuromyelitis optica), Discoid lupus, Dressler's syndrome, Endometriosis, Eosinophilic esophagitis, Eosinophilic fasciitis, Erythema nodosum, Experimental allergic encephalomyelitis, Evans syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis (GPA) (formerly called Wegener's Granulomatosis), Graves' disease, Guillain-Barre syndrome, Hashimoto's encephalitis, Hashimoto's thyroiditis, Hemolytic anemia, Henoch-Schonlein purpura, Herpes gestationis, Hypogammaglobulinemia, Idiopathic thrombocytopenia purpura (ITP), IgA nephropathy, IgG4-related sclerosing disease, Immunoregulatory lipoproteins, Inclusion body myositis, Interstitial cystitis, Juvenile arthritis, Juvenile diabetes (Type 1 diabetes), Juvenile myositis, Kawasaki syndrome, Lambert-Eaton syndrome, Leukocytoclastic vasculitis, Lichen planus, Lichen sclerosus, Ligneous conjunctivitis, Linear IgA disease (LAD), Lupus (SLE), Lyme disease, chronic, Meniere's disease, Microscopic polyangiitis, Mixed connective tissue disease (MCTD), Mooren's ulcer, Mucha-Habermann disease, Multiple sclerosis, Myasthenia gravis, Myositis, Narcolepsy, Neuromyelitis optica (Devic's), Neutropenia, Ocular cicatricial pemphigoid, Optic neuritis, Palindromic rheumatism, PANDAS (Pediatric Autoimmune Neuropsychiatric Disorders Associated with Streptococcus), Paraneoplastic cerebellar degeneration, Paroxysmal nocturnal hemoglobinuria (PNH), Parry Romberg syndrome, Parsonnage-Turner syndrome, Pars planitis (peripheral uveitis), Pemphigus, Peripheral neuropathy, Perivenous encephalomyelitis, Pernicious anemia, POEMS syndrome, Polyarteritis nodosa, Type I, II, & III autoimmune polyglandular syndromes, Polymyalgia rheumatica, Polymyositis, Postmyocardial infarction syndrome, Postpericardiotomy syndrome, Progesterone dermatitis, Primary biliary cirrhosis, Primary sclerosing cholangitis, Psoriasis, Psoriatic arthritis, Idiopathic pulmonary fibrosis, Pyoderma gangrenosum, Pure red cell aplasia, Raynauds phenomenon, Reactive Arthritis, Reflex sympathetic dystrophy, Reiter's syndrome, Relapsing polychondritis, Restless legs syndrome, Retroperitoneal fibrosis, Rheumatic fever, Rheumatoid arthritis, Sarcoidosis, Schmidt syndrome, Scleritis, Scleroderma, Sjogren's syndrome, Sperm & testicular autoimmunity, Stiff person syndrome, Subacute bacterial endocarditis (SBE), Susac's syndrome, Sympathetic ophthalmia, Takayasu's arteritis, Temporal arteritis/Giant cell arteritis, Thrombocytopeniarpura (TTP), Tolosa-Hunt syndrome, Transverse myelitis, Type 1 diabetes, Ulcerative colitis, Undifferentiated connective tissue disease (UCTD), Uveitis, Vasculitis, Vesiculobullous dermatosis, Vitiligo, or Wegener's granulomatosis (i.e., Granulomatosis with Polyangiitis (GPA).

For example, the modified therapeutic mRNAs described herein may be for use in methods of treating a neurodegenerative diseases. “Neurodegenerative disease” refers to a disease or condition in which the function of a subject's nervous system becomes impaired. Examples of neurodegenerative diseases that may be treated include Alexander's disease, Alper's disease, Alzheimer's disease, Amyotrophic lateral sclerosis, Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, frontotemporal dementia, Gerstmann-Straussler-Scheinker syndrome, Huntington's disease, HIV-associated dementia, Kennedy's disease, Krabbe's disease, kuru, Lewy body dementia, Machado-Joseph disease (Spinocerebellar ataxia type 3), Multiple sclerosis, Multiple System Atrophy, Narcolepsy, Neuroborreliosis, Parkinson's disease, Pelizaeus-Merzbacher Disease, Pick's disease, Primary lateral sclerosis, Prion diseases, Refsum's disease, Sandhoffs disease, Schilder's disease, Subacute combined degeneration of spinal cord secondary to Pernicious Anaemia, Schizophrenia, Spinocerebellar ataxia (multiple types with varying characteristics), Spinal muscular atrophy, Steele-Richardson-Olszewski disease, or Tabes dorsalis.

For example, the modified therapeutic mRNAs described herein may be for use in methods of treating a metabolic disease. “Metabolic disease” refers to a disease or condition in which a subject's metabolism or metabolic system (e.g., function of storing or utilizing energy) becomes impaired. Examples of metabolic diseases that may be treated include diabetes (e.g., type | or type II), obesity, metabolic syndrome, or a mitochondrial disease (e.g., dysfunction of mitochondria or aberrant mitochondrial function).

For example, the modified therapeutic mRNAs described herein may be for use in methods of treating an inflammatory disease. “Inflammatory disease” refers to a disease or condition characterized by aberrant inflammation (e.g. an increased level of inflammation compared to a control, such as a healthy person not suffering from a disease). Examples of inflammatory diseases include traumatic brain injury, arthritis, rheumatoid arthritis, psoriatic arthritis, juvenile idiopathic arthritis, multiple sclerosis, systemic lupus erythematosus (SLE), myasthenia gravis, juvenile-onset diabetes, diabetes mellitus type 1, Guillain-Barre syndrome, Hashimoto's encephalitis, Hashimoto's thyroiditis, ankylosing spondylitis, psoriasis, Sjogren's syndrome, vasculitis, glomerulonephritis, auto-immune thyroiditis, Behcet's disease, Crohn's disease, ulcerative colitis, bullous pemphigoid, sarcoidosis, ichthyosis, Graves ophthalmopathy, inflammatory bowel disease, Addison's disease, Vitiligo, asthma, allergic asthma, acne vulgaris, celiac disease, chronic prostatitis, inflammatory bowel disease, pelvic inflammatory disease, reperfusion injury, sarcoidosis, transplant rejection, n, interstitial cystitis, atherosclerosis, and atopic dermatitis.

For example, the modified therapeutic mRNAs described herein may be for use in methods of treating cancer. The modified therapeutic mRNAs provided herein may help to induce effective tumour-reactive T-cell responses to a tumour. When for use in treating cancer, the modified therapeutic mRNAs encode a tumour-associated epitope. The modified therapeutic mRNAs described herein may effectively help generate a population of immune cells, in particular of CD8+ effector T cells (also known as cytotoxic T lymphocytes (CTLs)). The immune cells induced by administration of the modified therapeutic mRNAs described herein may be reactive to the epitope, or epitopes, translated from the modified therapeutic mRNAs described herein. These immune cells are then primed for the killing of cancer cells that present the same or similar epitopes. Such modified therapeutic mRNAs for use in treating cancer may be referred to as “cancer vaccines” or “cancer immunotherapy vaccines”.

The medical uses and methods of treating cancer may include administering to a subject in need thereof a therapeutically effective amount of a modified therapeutic mRNAs as described herein.

The medical uses and methods of treatment described herein may be used in the treatment of a wide range of cancers. Tumours may be of mesenchymal or epithelial origin. Cancers include cancers of the colon, rectum, cervix, breast, lung, stomach, uterus, skin, mouth, tung, lips, larynx, kidney, bladder, prostate, brain, and blood cells. The medical uses and methods of treatment described herein may be used in the treatment of solid tumours.

Suitably, a cancer to be treated by a medical use or method of treatment described herein may be a solid tumour selected from but not limited to the group consisting of: pancreatic ductal adenocarcinoma, pancreatic cancer; breast cancer; melanoma; non-small cell lung cancer; small cell lung cancer; nasopharyngeal cancer; hepatocellular cancer; colorectal cancer; oesophageal cancer; gastric cancer; anal cancer; small intestine cancer; mesothelioma; kidney cancer; renal cell carcinoma; bladder cancer; prostate cancer; ovarian cancer; vulval cancer; cervical cancer; penile cancer; uveal melanoma; retinoblastoma; sarcoma; osteosarcoma; glioblastoma; adrenocortical carcinoma; neuroblastoma; Wilms tumour; endometrial cancer; and thyroid cancer.

In reference to cancer, the terms “treatment” and “treating” should be taken as encompassing therapy undertaken in order to prevent, slow down, or reduce an undesired physiological change or disorder, such as the growth, development or spread of cancer. Beneficial or desired results include but are not limited to the alleviation of symptoms, diminishment of the extent of disease, stabilized state of disease (which is to say, a disease that is not worsening), delay or slowing of disease progression, de-staging the tumour (e.g., changing from borderline resectable to amendable for surgical resection), amelioration or palliation of the disease state, and remission (either partial or total).

Treatment may bring about prolonged survival as compared to expected survival if not receiving treatment. Alternatively, or additionally, treatment may provide a patient with an improved standard of life as compared to that which would be expected if not receiving treatment.

For example, the modified therapeutic mRNAs described herein may be for use in methods of treating allergic disease. An “allergic disease” refers to a condition caused by hypersensitivity of the immune system to typically harmless substances in the environment. Allergic diseases include but are not limited to, asthma, hypersensitivity lung diseases, rhinitis, rhino-conjunctivitis, rhinosinusitis, atopic eczema, contact dermatitis, allergic conjunctivitis (intermittent and persistent), vernal conjunctivitis (hay fever), atopic keratoconjunctivitis, giant papillary conjunctivitis, urticaria (hives), angioedema, hypersensitivity pneumonitis, eosinophilic bronchitis, vasculitis, hypersensitivity vasculitis, antineutrophil cytoplasmic antibody (ANCA) associated vasculitis, Wegner's granulomatosis, Churg Strauss vasculitis, microscopic polyangiitis, temporal arteritis, celiac disease, mastocytosis, and anaphylaxis.

In particular, the modified therapeutic mRNAs described herein may encode a protein that includes an allergenic epitope. It will be apparent that in the majority of cases that the allergenic epitope is an epitope from or derived from an allergen that causes allergy symptoms or allergic reaction in a subject.

In some examples, the modified therapeutic mRNAs described herein that encode a protein that includes an allergenic epitope may be for use in methods of allergy immunotherapy (AIT). In some examples, subcutaneous allergy immunotherapy (SCIT). Modified therapeutic mRNAs for use in AIT and/or SCIT may be referred to as allergy vaccines.

In general, AIT comprises administering an allergen to the patient in order to treat an allergy to that allergen of the patient, i.e., reducing current or future immune response, such as an allergen-specific IgE response and/or histamine release by mastocytes and/or granulocytes induced by the allergen, and/or manifestation of clinical symptoms of allergy. Immunotherapy is conventionally carried out by repeatedly administering a mono-dose or incremental doses of an allergen to a patient in need thereof, thereby resulting in an adaptive immune response of the patient who becomes desensitised to the allergen.

During AIT or SCIT, increasing doses of the allergen or allergenic epitope are administered, followed by a maintenance dose for several years, with the goal of inducing immunological changes leading to symptom amelioration while on therapy, as well as sustained desensitization off AIT or SCIT (immune tolerance).

Typically, at the start of AIT or SCIT, subjects receive increasing doses of the allergen or allergenic epitope at weekly intervals over several weeks to months, under tightly monitored medical supervision. The gradual dose escalation enables tolerability to therapy and mitigates the risk of severe hypersensitivity reactions related to allergen administration.

For example, the modified therapeutic mRNAs described herein may be for use in methods of treating infectious diseases. “Infectious disease” refers to a disease which results from an infection. Infection is a condition caused by the invasion of an organism by a foreign agent (i.e., an infectious agent). Infectious agents include but are not limited to, bacteria, fungi, viruses, viroids, nematodes (e.g., parasites such as roundworms and pinworms), anthropods (e.g., mites, fleas, lice, ticks), and macroparasites (e.g., tapeworms). Common infectious diseases include bacterial and viral infections. The modified therapeutic mRNAs described herein, when for use in methods of treating infectious disease, may include an epitope or antigen from or derived from the pathogen causing the infectious disease.

The modified therapeutic mRNAs described herein may be particularly useful in prevention or treatment of infectious diseases caused by intracellular pathogens. For example, viruses (e.g., CMV, HIV, SARS viruses, such as Covid 19, coronaviruses), bacteria (e.g., Listeria, Mycobacteria, Salmonella (e.g., S. typhi) enteropathogenic Escherichia coli (EPEC), enterohaemorrhagic Escherichia coli (EHEC), Yersinia, Shigella, Chlamydia, Chlamydophila, Staphylococcus, Legionella), protozoa (e.g., Taxoplasma), fungi, and intracellular parasites (e.g., Plasmodium (e.g., P. vivax, P. falciparum, P. ovale, and P. malariae). The compositions and formulations described herein may reduce humoral response against an immunogenic immunomodulator, including epitopes derived from such intracellular pathogens and increase cellular mediated response.

“Treatment” in relation to infectious diseases refers to any administration of a modified therapeutic mRNA as described herein that partially or completely alleviates, ameliorates, relieves, inhibits, delays the onset of, reduces the severity of and/or reduces the incidence of one or more symptoms or features of an infectious disease or the predisposition toward the disease. Such treatment may be of a subject who does not exhibit signs of the relevant disease, and/or of a subject who exhibits only early signs of the disease. Alternatively or additionally, such treatment may be of a subject who exhibits one or more established signs of the relevant disease. As such the term “treating” in reference to infectious diseases refers to the vaccination of a subject. “Prevention” refers to a delay of onset of an infectious disease. Prevention may be considered complete when onset of an infectious disease or disorder has been delayed for a predefined period of time.

In some examples, the modified therapeutic mRNAs described herein may be for use as a vaccine. As such, in some examples provided herein are immunogenic compositions comprising a modified therapeutic mRNA as described. In some examples, the compositions are vaccine compositions. The terms “immunogenic composition” and “immunological composition” and “immunogenic or immunological composition” refer to compositions that elicit an immune response against an antigen or immunogen after administration into a subject. The terms “vaccine” and “vaccine composition” refers to compositions that induce a protective immune response against the antigen of interest or which efficaciously protects against the antigen; for instance, after administration to the subject, elicits a protective immune response against the targeted antigen or immunogen.

In some examples, the methods of preventing and/or treating infectious disease is a method of vaccination. “Vaccination” refers to the administration of a modified therapeutic mRNA as described herein intended to generate an immune response, for example to a disease-causing pathogen. Vaccination can be administered before, during, and/or after exposure to a disease-causing pathogen, and in some examples, before, during, and/or shortly after exposure to the agent. In some examples, vaccination includes multiple administrations, appropriately spaced in time, of a modified therapeutic mRNA as described herein.

In some examples, the modified therapeutic mRNAs may be for use in vaccination against a virus. For example, for use in vaccination of a subject against viruses of the retroviridae, orthmyxoviridae, paramyxoviridae, arenaviridae, bunyaviridae, flaviviridae, filoviridae, togaviridae, picornaviridae, caliciviridae and coronaviridae families. Examples of such viruses include, but are not limited to, adenovirus, rhinovirus, hepatitis, immunodeficiency virus, polio, measles, Ebola, Coxsackie, Rhino, West Nile, small pox, encephalitis, yellow fever, Dengue fever, influenza (including human, avian, and swine), lassa, lymphocytic choriomeningitis, junin, machuppo, guanarito, hantavirus, Rift Valley Fever, La Crosse, California encephalitis, Crimean-Congo, Marburg, Japanese Encephalitis, Kyasanur Forest, Venezuelan equine encephalitis, Eastern equine encephalitis, Western equine encephalitis, severe acute respiratory syndrome (SARS), parainfluenza, respiratory syncytial, Punta Toro, Tacaribe and pachindae.

In some examples, the virus is an influenza (including human, avian, and swine) or a severe acute respiratory syndrome (SARS) virus. In some examples, the virus is a coronaviridae virus. In some examples, the virus is Covid-19.

As used herein, the terms “treat”, “treating” and “treatment” generally are taken to include an intervention performed with the intention of preventing the development or altering the pathology of a condition, disorder or symptom (e.g., an allergic disease, infectious disease, etc.). Accordingly, “treatment” refers to both therapeutic treatment and prophylactic or preventative measures (such as vaccination), wherein the object is to prevent or slow down (lessen) the targeted condition, disorder or symptom. “Treatment” therefore encompasses a reduction, slowing or inhibition of disease symptoms, for example, at least 5%, 10%, 20%, 30%, 40%, 50%, 60%, 70%, 80%, 90% or 100% when compared to before treatment.

As used herein the term “subject” generally refers to an individual, e.g., a human, having or at risk of having a specified condition, disorder or symptom. The subject may be a patient, i.e., a subject in need of treatment in accordance with the invention. The subject may have received treatment for the condition, disorder or symptom. Alternatively, the subject has not been treated prior to treatment in accordance with the present invention.

The modified therapeutic mRNAs described herein generally can be administered to the subject by any conventional route, including injection or by gradual infusion over time. The administration may, for example, by intramuscular, intravascular, intracavity, intracerebral, intralesional, rectal, subcutaneous, intradermal, epidural, intrathecal, and percutaneous administration.

The methods of treatment and medical uses described herein may provide modified therapeutic mRNAs as described herein to a recipient via any suitable route of administration. The modified therapeutic mRNAs can be administered via any desired route of administration. The modified therapeutic mRNAs, or medical uses, may make use of a route of administration selected from the group consisting of: intravenous (iv) administration; subcutaneous (sc) administration; intramuscular (im) administration; intradermal (id) administration; sublingual (sl) administration; and intranasal administration.

The skilled person will be able to determine suitable forms of the modified therapeutic mRNAs of the invention for use with the desired route of administration.

In some examples, the modified therapeutic mRNAs as described may be administered via a route selected from intratumoral, inhalation, or intracardiac injection.

The modified therapeutic mRNAs described herein are for administration in an effective amount. An “effective amount” is an amount that alone, or together with further doses, produces the desired (therapeutic or non-therapeutic) response. The effective amount to be used will depend, for example, upon the therapeutic (or non-therapeutic) objectives, the route of administration, and the condition of the patient/subject. For example, the suitable dosage of a modified therapeutic mRNA of the invention for a given patient/subject will be determined by the attending physician (or person administering the composition), taking into consideration various factors known to modify the action of the modified therapeutic mRNAs of the invention for example severity and type of disease, body weight, sex, diet, time and route of administration, other medications and other relevant clinical factors. The dosages and schedules may be varied according to the particular condition, disorder or symptom of the overall condition of the patient/subject. Effective dosages may be determined by either in vitro or in vivo methods.

### Compositions

In some examples, the modified therapeutic mRNAs as described herein, are formulated as compositions. In some examples, the modified therapeutic mRNAs as described herein, may be provided as part of a pharmaceutical formulation or composition. Advantageously, such formulations may be administered to a human subject in need thereof (as described elsewhere herein).

The compositions, including the modified therapeutic mRNAs as described, may include delivery agents specific to the administration and delivery of modified therapeutic mRNA to a subject. For example, the compositions may include modified therapeutic mRNA encapsulated in lipids, polymers, or dendrimers. In some examples, the compositions may include cell-penetrating peptides that may or may not be linked or may link in use (i.e. covalently or non-covalently) to the modified therapeutic mRNA or the translation product thereof. For example, modified therapeutic mRNAs may be delivered to a target tissue or cell by the use of lipid nanoparticles, autologous T cells, CAR-T cells, plasmid DNA, modified CD34+hHSPCs, cytotoxic T lymphocytes, or T cells. For example, see Kowalski P S, Rudra A, Miao L, Anderson D G. Delivering the Messenger: Advances in Technologies for Therapeutic mRNA Delivery. Mol Ther. 2019; 27 (4): 710-728. doi: 10.1016/j.ymthe.2019.02.012 and Qin S, Tang X, Chen Y, et al. mRNA-based therapeutics: powerful and versatile tools to combat diseases. Signal Transduct Target Ther. 2022; 7 (1): 166. Published 2022 May 21. doi: 10.1038/s41392-022-01007-w.

A pharmaceutical formulation and the compositions described herein may comprise a modified therapeutic mRNA as described herein, along with a pharmaceutically acceptable excipient, adjuvant, diluent and/or carrier.

Compositions and formulations may routinely contain pharmaceutically acceptable concentrations of salt, buffering agents, preservatives, compatible carriers, supplementary immune potentiating agents such as adjuvants and cytokines and optionally other therapeutic agents or compounds.

As used herein, “pharmaceutically acceptable” refers to a material that is not biologically or otherwise undesirable, i.e., the material may be administered to an individual along with the selected modified therapeutic mRNA without causing any undesirable biological effects or interacting in a deleterious manner with any of the other components of the pharmaceutical formulation in which it is contained.

Excipients are natural or synthetic substances formulated alongside an active ingredient (e.g. a modified therapeutic mRNA as provided herein), included for the purpose of bulking-up the formulation or to confer a therapeutic enhancement on the active ingredient in the final dosage form, such as facilitating drug absorption or solubility. Excipients can also be useful in the manufacturing process to aid in the handling of the active substance concerned, such as by facilitating powder flowability or non-stick properties, in addition to aiding in vitro stability, such as prevention of denaturation over the expected shelf life. Pharmaceutically acceptable excipients are well known in the art. A suitable excipient is, therefore, easily identifiable by one of ordinary skill in the art. By way of example, suitable pharmaceutically acceptable excipients include water, saline, aqueous dextrose, glycerol, ethanol, and the like.

Adjuvants are pharmacological and/or immunological agents that modify the effect of other agents in a formulation. Pharmaceutically acceptable adjuvants are well-known in the art. A suitable adjuvant is, therefore, easily identifiable by one of ordinary skill in the art. Merely by way of example, a pharmaceutical formulation may comprise an adjuvant selected from the group consisting of: AS03; AddaS03; AS04; MF59; AddaVax; Poly I: C; R848; Cpg; virus-like particles; virosomes; MPL; and flagellin protein.

Diluents are diluting agents. Pharmaceutically acceptable diluents are well-known in the art. A suitable diluent is, therefore, easily identifiable by one of ordinary skill in the art.

Carriers are non-toxic to recipients at the dosages and concentrations employed and are compatible with other ingredients of the formulation. The term “carrier” denotes an organic or inorganic ingredient, natural or synthetic, with which the active ingredient is combined to facilitate the application. Pharmaceutically acceptable carriers are well known in the art. A suitable carrier is, therefore, easily identifiable by one of ordinary skill in the art.

### Methods of Producing and Uses

Also provided herein are methods of producing the modified therapeutic mRNAs as described. The methods involve providing a therapeutic mRNA. Providing therapeutic mRNAs may be done passively or actively. That is to say that the therapeutic mRNA may be provided by a third party. For example, purchased or obtained from a third party that actively produced the mRNA. In some examples, providing a therapeutic mRNA may include synthesising or purifying the therapeutic mRNA. Methods of synthesising mRNA and therapeutic mRNAs are known in the art. For example, the therapeutic mRNA may be synthesised using methods such as IVT, as described above. Other methods will be known to those skilled in the art, such as those described in Qin S, Tang X, Chen Y, et al. mRNA-based therapeutics: powerful and versatile tools to combat diseases. Signal Transduct Target Ther. 2022; 7 (1): 166. Published 2022 May 21. doi: 10.1038/s41392-022-01007-w.

In some examples, a nucleic acid template encoding the therapeutic mRNAs as described may be provided or produced. For example, a DNA template suitable for transcription of the therapeutic mRNA.

The provided therapeutic mRNA is then analysed to identify frameshift nucleic acid sequences within the nucleic acid sequence of the therapeutic mRNA. Identifying frameshift nucleic acid sequences may be done by any suitable method. When the sequence of the mRNA is not known, identifying may include sequencing the mRNA. RNA sequencing may include the use of methods such as amplification (PCR) based methods (reverse transcriptase PCR (RT-PCR) and quantitative reverse transcriptase PCR (qRT-PCR), or RNAseq (next generation sequencing, also referred to as second generation sequencing or massively parallel sequencing). Methods of DNA sequencing are known and include similar methods as those used for determining mRNA sequences.

Next Generation Sequencing (NGS) (second generation sequencing or massively parallel sequencing; Mardis, E. R. (2008). As there are many NGS technologies available, there are small differences in the methodology for RNA sequencing. The following is a description of how RNA sequencing using NGS works in general. Total RNA is extracted from the sample of interest using a common RNA extraction method. Post-extraction processes can be used to enrich the RNA sample. Complimentary DNA (cDNA) is then synthesised using extracted RNA. cDNA is then used as the template for RNA sequencing. NGS uses variations of sequencing by synthesis (SBS) chemistry (Fuller, C. W., et al. (2009). The challenges of sequencing by synthesis. Nature biotechnology, 27 (11), 1013-1023). With cDNA as a template, new nucleotide fragments, known as reads, are synthesised base by base, with each incorporated base recorded during sequencing (Fuller, 2009). The data output from RNA sequencing is a list of all the reads generated, and their sequence (Fuller, 2009 and Metzker, 2010). This data undergoes quality assessment (Patel, R. K., & Jain, M. (2012). NGS QC Toolkit: a toolkit for quality control of next generation sequencing data. PloS one, 7 (2), e30619).

If the sequence of the therapeutic mRNA is known, identifying may include the use of sequence analysis software that may identify frameshift nucleic acid sequences as described above. In some examples, the mRNA sequence may be analysed manually by referring to frameshift nucleic acid sequences already described in the art.

Once frameshift nucleic acid sequences have been identified, the therapeutic mRNA is modified to include a synonymous mutation in one or more of the identified frameshift nucleic acid sequences. Modification of the mRNA sequence may be achieved by any known nucleic acid modification method, such as random mutagenesis, chemical mutagenesis, site-directed mutagenesis or gene editing techniques. In some examples, the modification is introduced into a template for the production of a modified therapeutic mRNA. For example, a DNA template used for production (e.g. IVT) of the mRNA.

As such, there are also provided herein IVT templates that encode a modified therapeutic mRNA as described herein. For example, DNA molecules encoding modified therapeutic mRNA as described herein.

Editing and/or mutagenesis technologies are well-known in the art. As well, introduction may be accomplished in any manner known in the art, including: introgression, transgenic, or site-directed nucleases (SDN). Particularly, the modification to the DNA sequence is introduced by way of site-directed nuclease (SDN). More particularly, the SDN is selected from: meganuclease, zinc finger, transcription activator-like effector nucleases system (TALEN) or Clustered Regularly Interspaced Short Palindromic Repeats system (CRISPR) system. SDN is also referred to as “genome editing”, or genome editing with engineered nucleases (GEEN). This is a type of genetic engineering in which DNA is inserted, deleted or replaced using engineered nucleases that create site-specific double-strand breaks (DSBs) at desired locations in the DNA. The induced double-strand breaks are repaired through nonhomologous end-joining (NHEJ) or homologous recombination (HR), resulting in targeted mutations (‘edits’). Particularly SDN may comprise techniques such as: Meganucleases, Zinc finger nucleases (ZFNs), Transcription Activator-Like Effector-based Nucleases (TALEN) (Feng et al. 2013 Cell Res. 23, 1229-1232, Sander & Joung Nat. Biotechnol. 32, 347-355 2014), and the Clustered Regularly Interspaced Short Palindromic Repeats (CRISPR-Cas) system. Gene editing may also be achieved by SDN-2. SDN-2 is similar to SDN, but also provides a small nucleotide template complementary to the area of the break. The template contains one or more sequence modifications to the DNA, which are incorporated to create the mutation to the target DNA.

Once the one or more synonymous mutations have been introduced into the mRNA or into a template for the production of the mRNA, the mRNA, including the one or more synonymous mutations, is produced. For example, using methods such as IVT as described herein.

The modified therapeutic mRNAs provided herein may be for use as translation templates for in vitro or ex vivo production of proteins. The increased fidelity and/or efficiency of translation provided by introduction of the synonymous mutations as described herein may provide for improved methods of in vitro translation and therefore improved methods of in vitro protein synthesis. Given the properties of the modified therapeutic mRNAs described herein (e.g. reduced out-of-frame translation, reduced immunogenicity, improved translation fidelity and/or improved translation efficiency), the modified therapeutic mRNAs may, for a number of different uses and methods.

For example, provided here is a method of reducing off-target immunogenicity to a modified therapeutic mRNA and/or translation product thereof, which includes producing a modified therapeutic mRNA as described. After production, the modified therapeutic mRNA is administered to a subject in need thereof as described herein.

Once administered, the modified therapeutic mRNA, including the one or more synonymous mutations, is translated within the subject. The translation products comprise lower levels of out-of-frame translation products which leads to lower levels of immunogenicity to the translation products in total in comparison to a therapeutic mRNA, not including the synonymous mutation.

For example, provided here is a method of reducing out-of-frame translation of a modified therapeutic mRNA. The method includes producing a modified therapeutic mRNA, including the one or more synonymous mutations as described herein. After production of the modified therapeutic mRNA, including the one or more synonymous mutations, the mRNA is translated.

The inclusion of the one or more synonymous mutations may lead to reduced stalling and reduced levels of out-of-frame translation (and ergo production of out-of-frame translation products). Such a method may also increase the fidelity of translation. Therefore, also provided are methods of increasing translation fidelity of a modified therapeutic mRNA including the one or more synonymous mutations as described herein.

In some examples, translation of the modified therapeutic mRNA including the one or more synonymous mutations may be done in vitro, ex vivo or in vivo.

“Ex vivo” generally refers to activities that take place outside an organism, such as experimentation or measurements done in or on living tissue in an artificial environment outside the organism, preferably with minimum alteration of the natural conditions.

The use of in vitro translation has a variety of applications, including the rapid identification of gene products (e.g., proteomics), localization of mutations through synthesis of truncated gene products, protein folding studies, and incorporation of modified or unnatural amino acids for functional studies. All of these may be improved by the use of mRNAs that have been modified as described herein.

Methods of in vitro translation are well known in the art. For example, methods may include the use of in vitro translation systems such as rabbit reticulocyte lysate, wheat germ extract, or E. coli cell-free systems.

Unless defined otherwise herein, all technical and scientific terms used herein have the same meaning as commonly understood by one of ordinary skill in the art to which this invention pertains. For example, Singleton and Sainsbury, Dictionary of Microbiology and Molecular Biology, 2d Ed., John Wiley and Sons, NY (1994); and Hale and Marham, The Harper Collins Dictionary of Biology, Harper Perennial, NY (1991) provide those of skill in the art with a general dictionary of many of the terms used in the invention. Although any methods and materials similar or equivalent to those described herein find use in the practice of the present invention, the preferred methods and materials are described herein. Accordingly, the terms defined immediately below are more fully described by reference to the Specification as a whole. Also, as used herein, the singular terms “a”, “an,” and “the” include the plural reference unless the context clearly indicates otherwise. Unless otherwise indicated, nucleic acids are written left to right in 5′ to 3′ orientation; amino acid sequences are written left to right in amino to carboxy orientation, respectively. It is to be understood that this invention is not limited to the particular methodology, protocols, and reagents described, as these may vary, depending upon the context they are used by those of skill in the art.

Aspects of the invention are demonstrated by the following non-limiting examples.

### EXAMPLES

### Summary

In vitro-transcribed (IVT) mRNAs are modalities that can combat human disease, exemplified by their use as vaccines for SARS-COV-2. IVTmRNAs are transfected into target cells, translated into recombinant protein, and the biological activity or immunogenicity of the encoded protein exerts an intended therapeutic effect ¹⁻³. Modified ribonucleotides are commonly incorporated into therapeutic IVTmRNAs to decrease their innate immunity ^{4,5}, but their effects on mRNA translation fidelity have not been fully explored. Here it is demonstrated that (N)1-methylpseudouridine incorporation into mRNA results in +1 ribosomal frameshifting in vitro and in vivo, and that cellular immunity in mouse and humans to +1 frameshifted products from BNT162b2 mRNA translation occurs after vaccination. The +1 ribosome frameshifting observed is a consequence of (N)1-methylpseudouridine-induced ribosome stalling during IVTmRNAmRNA translation, with frameshifting occuring at slippery sequences. However, it is demonstrated that synonymous mutations targeting such slippery sequences provides an effective strategy to reduce the production of frameshifted products. Overall these data increase the understanding of how modified ribonucleotides affect the fidelity of mRNA translation, and although there are no adverse outcomes reported from mRNA-based SARS-CoV-2 vaccine in humans, highlight potential off-target effects for future such therapeutics, demonstrating the requirement for additional sequence optimisation.

### Materials and Methods

### Ethics Statement

Animal experiments were licensed by the UK Home Office according to the Animals Scientific Procedures Act 1986 (License pp 6047,951) and approved by local ethics committees from the University of Cambridge. Human sample collection and analysis was conducted in accordance with the principles of Good Clinical Practice and following approved protocols of the NIHR National Bioresource. Samples were collected with the written informed consent of all study participants under the NIHR National BioResource-Research Tissue Bank (NBR-RTB) ethics (REC: 17/EE/0025) and from the PITCH study. PITCH is a sub-study of the SIREN study, which was approved by the Berkshire Research Ethics Committee, Health Research 250 Authority (IRAS ID 284460, REC reference 20/SC/0230), with PITCH recognised as a sub-study on 2nd December 2020. SIREN is registered with ISRCTN (Trial ID: 252 ISRCTN11041050). Some participants were recruited under aligned study protocols. In Liverpool, some participants were recruited under the “Human immune responses to acute virus infections” Study (16/NW/0170), approved by North West-Liverpool Central Research Ethics Committee on 8th March 2016, and amended on 14th September 2020 and 4th May 2021. In Oxford, participants were recruited under the GI Biobank Study 16/YH/0247, approved by the research ethics committee (REC) at Yorkshire & The Humber-Sheffield Research Ethics Committee on 29th July 2016, which has been amended for this purpose on 8th June 2020. The study was conducted in compliance with all relevant ethical regulations for work with human participants, and according to the principles of the Declaration of Helsinki (2008) and the International Conference on Harmonization (ICH) Good Clinical Practice (GCP) guidelines. Written informed consent was obtained for all participants enrolled in the study.

Plasmids and mRNA Synthesis

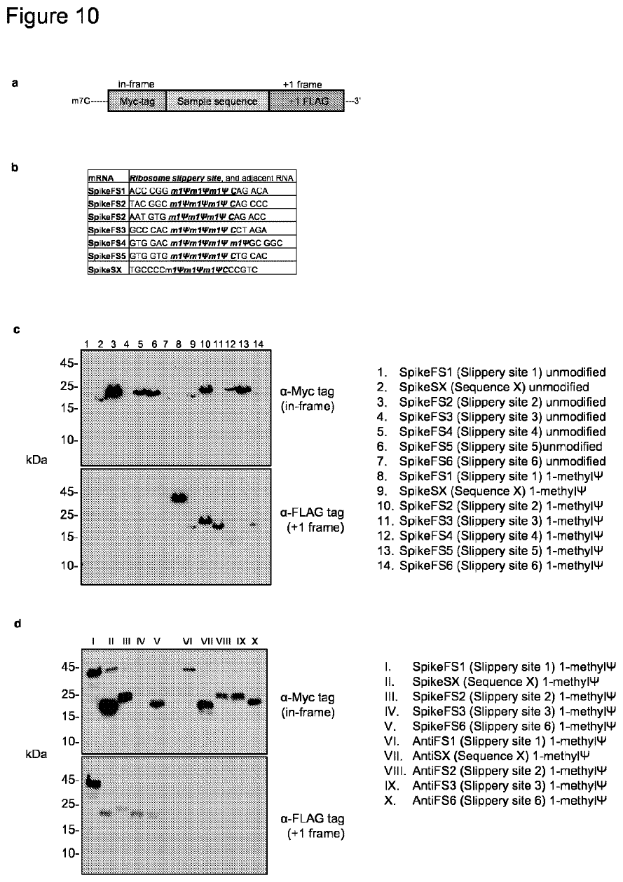

Phusion High-Fidelity DNA polymerase reagents were obtained from New England Biolabs (Ipswich, USA). In-frame WTFluc template DNA was produced by Xbal digest of PUCK100Fluc, including an 80 nt polyA tail 30. Fluc +1FS and Fluc-1FS template DNAs were produced by overlap extension PCR of pUCK100Fluc using FlucFLAG_F, Fluc-1FS_R for Fluc-1FS NFluc, Fluc-1FS_F, and Fluc_R for Fluc-1FS CFluc, or FlucFLAG_F, Fluc +1FS_R for Fluc +1FS NFluc, Fluc +1FS_F, and Fluc_R for Fluc +1FS CFluc. PCR products were re-inserted into pUCK100 using Ncol and Nhel, and linear template DNA was produced by Xbal digest. A206G, T187C, and T208C mRNA, and all mRNAs in FIG. 10 were transcribed from custom genes subcloned into pUC57T7 (Genscript Biotech Corporation, New Jersey, USA) and linear template DNA was produced by BamHI digest or Xbal digest. Fluc +1FS2 mRNA was produced from Fluc +1FS template DNA subcloned into pUC57T7 and linearised by

BamHI. U*187C/U*208C template DNA was produced by overlap extension PCR and reinsertion into pUC57. In vitro transcription was performed using TranscriptAid T7 High Yield Transcription Kit (Thermo Scientific K0441). UTP and CTP were substituted where required for 5-methoxyUTP, (N)1-methylpseudoUTP, or 5-methylCTP. Modified nucleotides were obtained from Trilink Biotechnologies (San Diego, USA). Transcripts were 5′-capped using Vaccinia Capping System (NEB M2080S) and purified by phenol/chloroform extraction and G50 size exclusion. Transcripts were quantified using a Nanodrop ND2000 spectrophotometer (Thermo Scientific) and stored at −80° C.

### RNA Gel Electrophoresis

Samples were heated in formamide/bromophenol blue/xylene cyanol dye for 3 minutes at 95° C., cooled for 2 minutes on ice, and resolved on a 1% agarose formaldehyde MOPS-acetate gel for 90 minutes at 90 V. The gel was stained in 0.5 μg/ml ethidium bromide for 1 hour, bathed in distilled water for 1 hour and visualised by UV transillumination.

Cell culture and mRNA transfection

Hela cells were a gift from the Proudfoot lab, University of Oxford. Cells were grown in DMEM (Gibco 41966029), supplemented with 10% FBS at 37° C., 5% CO₂. Approximately 16 hours before transfection, cells were seeded at 0.2×10⁶/ml in 6-well plates. 10 minutes before transfection, medium was changed to OptiMEM (Gibco 31985062), after which cells were transfected with 4 μmol Fluc +1FS mRNA/Lipofectamine-2000 (Invitrogen 11668019). After 4 hours transfection, OptiMEM was replaced with DMEM, cells were cultured a further 4 hours, and lysed in Passive Lysis Buffer (Promega E1941). Lysates were centrifuged (10,000g, 5 minutes) and luciferase activity determined in supernatants using the Luciferase Assay System (Promega E4550) and GloMax multi-well plate luminometer (Promega).

### In Vitro Translation

IVTmRNAs were translated using the Flexi® Rabbit Reticulocyte Lysate System using nuclease-treated RRL (Promega L4540). For co-translational labelling, 0.33 μl translation-grade [³⁵S]-Methionine (Hartman Analytic KSM-01) and 0.67 μl amino acids minus methionine (Promega L996A) were used per 15 μl reaction. Unlabelled products were produced with 1 μl total (unlabelled) amino acids (Promega L4461). The quantity of IVTmRNA was 50 nM and paromomycin (Sigma Aldrich P9297) included where described at 100 μM. Creatine phosphate (Roche 10621714001), creatine kinase (Roche 21778721), potassium acetate (Sigma Aldrich P1190), and magnesium acetate (Sigma Aldrich M5661) were included at 10 mM, 25 μg/ml, 50 mM, and 0.5 mM, respectively 36. Reactions were incubated at 30° C. for the indicated time and moved to ice, to which 10 μl of RNase A/T1/Benzonase were added and incubated for 10 minutes. Luciferase activity was determined using the Luciferase Assay System (Promega E4550) and measured using a GloMax multi-well plate luminometer (Promega). For western blotting, 2X reducing LDS PAGE buffer was mixed with each sample, which was heated 70° C. for 10 minutes. Cooled samples were resolved on NuPAGE™ 12%, Bis-Tris, 1.0 mm, Mini Protein Gels (Invitrogen NP0342BOX). For analysis in FIG. 10, samples were resolved on Novex 10 to 20%, Tricine, 1.0 mm, Mini Protein Gels (Invitrogen EC66255BOX). The resolved products were transferred to nitrocellulose membrane and probed using anti-FLAG M2 antibody (Sigma Aldrich F1804), anti-Myc tag antibody [9E10] (AbCam Ab32), anti-mouse-HRP antibody (Dako P0447), and detected with Clarity Western ECL substrate (Bio-Rad 1705060).

### Peptide LC-MS/MS Analysis

IVT mRNA was translated as above. After RNA digestion, translation products were immunoprecipitated using anti-FLAG magnetic agarose beads (Pierce) overnight at 4° C. Beads were washed twice in PBS, once in water, eluted in LDS PAGE buffer, and resolved on a NuPAGE™ 4-12%, Bis-Tris, 1.5 mm, Mini Protein Gel (NP0335BOX). The gel was stained with coomassie dye and the region between ~60 kDa and 75 kDa (Precision Plus Protein™ All Blue Prestained Protein Standard, Bio-rad) was excised and processed for mass spectrometry analysis as previously described 37. Briefly, the excised gel slice was cut into 1 mm pieces and placed in an 1.5 ml microtube. Coomassie staining was removed by incubating alternatively with a mixture of 25 mM ammonium bicarbonate and acetonitrile (2:1) and 25 mM ammonium bicarbonate. Each 15 min incubation at 37° C. was repeated until gel pieces were completely distained. Reduction and alkylation of cysteines as done by first incubating with a fresh 10 mM final concentration of dithiothreitol in 25 mM ammonium bicarbonate at 60° C. for 60 min and then changing the solution to 60 mM final concentration of iodoacetamide in 25 mM ammonium bicarbonate and incubating for an addition 45 min at room temperature in the dark. After dehydrating the gel pieces with acetonitrile, trypsin solution was added (10 ng/uL in 25 mM ammonium bicarbonate) until gel pieces were completely covered. Digestion was carried at 37° C. over 16 h. Trypsin inactivation was done by adding formic acid to a final concentration of 1% (v/v). Peptides were then extracted d by sequential incubations with water:acetonitrile:formic acid (50:49:1% (v/v)) and (80:19:1). Extracted peptides were pooled and dried to completion and resuspended in water:acetonitrile (97:3% (v/v)) with 0.1% (v/V) TFA for mass spectrometry analysis.

### Mass Spectrometry Analysis

In-gel digests were analysed using an Ultimate 3000 RSLC™ nano system (Thermo Scientific, Hemel Hempstead) coupled to an Orbitrap Eclipse™ mass spectrometer (Thermo Scientific). The sample was loaded onto the trapping column (Thermo Scientific, PepMap100, C18, 300 μm×5 mm), using partial loop injection, for three minutes at a flow rate of 15 μL/min with 0.1% (v/v) FA in 3% acetonitrile. Peptides were separated on the analytical column (Easy-Spray C18 75 μm×500 mm 2 μm column) at a flow rate of 300 nL min-1 using a gradient of 97% A (0.1% formic acid) 3% B (80% acetonitrile 0.1% formic acid) to 25% B over 50 minutes, then to 40% B for additional 6 minutes, then to 90% B for another 2 minutes which remained at 90% B for 12 minutes, percentage of B was then lowered to 3.8% to allow the column to re-equilibrate for 15 minutes before next injection. Data was acquired using two FAIMS cv's (−50v, −70v). For each FAIMS experiment (maximum cycle time of 1.5s per experiment) data was acquired in data-dependent mode and MS1 consisted of a 120,000 resolution full-scan MS scan (AGC set to 100% (4e5 ions) with a maximum fill time of 50 ms) using a mass range of 380-1500 m/z. The intensity MS2 trigger threshold was set to 5.0e3 and to avoid repeated selection of peptides for MSMS the experiment used a 40 second dynamic exclusion window. MS/MS was performed on the orbitrap using 30,000 resolution (AGC set to 100% (5e4 ions) with a maximum fill time of 54 ms). 32% HCD collision energy was used to fragment the peptides and an isolation window of 1.2 was used.

### Proteome Discoverer v2.5 Analysis

Raw data were imported and data processed in Proteome Discoverer v2.5 (Thermo Fisher Scientific). The raw files were submitted to a database search using Proteome Discoverer with SequestHF against the Homo sapiens database containing human protein sequences from UniProt/Swiss-Prot, appended with Firefly luciferase, common contaminant proteins (several types of human keratins, BSA and porcine trypsin). The spectra identification was performed with the following parameters: MS accuracy, 10 p.p.m.; MS/MS accuracy of 0.02 Da; up to two missed cleavage sites allowed; carbamidomethylation of cysteine; and oxidation of methionine as variable modifications. An iteractive workflow was used in the processing step. After the first Sequest HT search, the Inferis Rescoring node was used and spectrum with confidence worse than high were re-submitted for a second Sequest HT search using additional dynamic modifications (N,Q deamidation; N-terminal pyroglutamate; methionine loss and acetylation). Peptides were assigned to their respective reading frame from Fluc +1FS mRNA by inspection. Percolator node was used for false discovery rate estimation and only rank 1 peptide identifications of high confidence (FDR<1%) were accepted.

### RNA-Seq Analysis

RNA-seq libraries were prepared from 1 μg IVTmRNA using NextFlex Rapid Directional RNA-seq kit 2.0 (Perkin Elmer), according to the manufacturer's protocol. Libraries were amplified by 6 PCR cycles and purified by PAGE. Sequencing was performed using an Illumina MiSeq at the Department of Biochemistry DNA sequencing facility, University of Cambridge (1×150 cycles V3). Reads were aligned with STAR 2.7.4a 38. Insertions and deletions per reference nucleotide were mapped from high quality reads (QC score>35) filtered for partial alignments and normalised to read depth. Insertion/deletion plots display the average mutation frequency for n=3 replicated RNA-seq experiments.

### SDS-PAGE Autoradiography

IVTmRNAs were translated in nuclease-treated RRL (Promega) and products co-translationally labelled as described above for 30 minutes. For peptidyl-tRNA analysis, samples were aliquoted into duplicates, to which 2.5 μl RNase A/T1/benzonase or water were added and incubated for a further 10 minutes to obtain RNase+/−samples. 2X LDS PAGE buffer was mixed to each sample, which was heated 70° C. for 10 minutes. Cooled samples were resolved on NuPAGE™ 12%, Bis-Tris, 1.0 mm, Mini Protein Gels (Invitrogen NP0342BOX). The resolved gels were fixed in 10% methanol/acetic acid for 45 minutes, and dried at 80° C. for 2 hours using a Fisher gel dryer system. Images were obtained by autoradiography using a Typhoon FLA 9000 and storage phosphor screens (GE Healthcare).

### Incorporated [³⁵S]-Methionine Quantification

IVTmRNAs were translated in nuclease-treated RRL (Promega) and products co-translationally labelled for 2 hours, as described above. [³⁵S]-Met incorporation was assayed according to the manufacturer's protocol. Briefly, after RNA digestion, reactions were incubated for 10 minutes in 1 M NaOH. Polypeptides were precipitated in 5% TCA, collected on Whatman glass fibre filters, and washed three times with 5% TCA and once with acetone. The dried filters were immersed in 2 ml EcoScint liquid scintillation cocktail (National Diagnostics) and counted in a Tri-Carb 4910 TR liquid scintillation counter (PerkinElmer). Incorporated [³⁵S]-Met was determined from cpm of precipitated polypeptides per cpm of unwashed filters for each reaction (total cpm).

### Mouse Immunisation

C57BL/6J mice (wild type, WT) were purchased from Charles Rivers laboratories. Mice were intramuscularly injected with two doses of 10 μg of BNT162b2 or left untreated. Spleens were obtained at day 8 post-vaccination and cell suspensions were prepared. Briefly, spleens were mashed with a syringe plunge and filtered through 70 μm cell strainers. Red blood cells were lysed with RBC lysing buffer (155 mM NH₄Cl, 12 mM NaHCO₃, 0.1 mM EDTA), before counting and cryopreserving prior to ELISpot assays.

### IFNγ ELISpot

Human IFNγ ELISpot assays were performed as previously described using the Human IFN-γ ELISpot PLUS kit (ALP) (MabTech 3420-4APT) 39. In-frame Spike peptide pools and +1FS Spike peptide pools were obtained from Mimotopes (Melbourne, Australia) (SEQ ID NOs: 10 -290. Spike S1+S2 peptide pools were as described previously 39. Briefly, cryopreserved PBMCs were thawed in RPMI 1640 medium supplemented with 1% (v/v) Penicillin/Streptomycin (Sigma), containing 0.01% (v/v) Benzonase Nuclease (Merck). PBMCs were washed and then incubated for 1-2 hours at 37° C., 5% CO₂ in RPMI1640 medium, 10% (v/v) Human AB Serum and 1% (v/v) Penicillin/Streptomycin. Pre-coated IFNγ ELISpot 96-well plates (MabTech 3420-4APT-2) were washed three times with PBS then blocked with RPMI 1640 medium/10% (v/v) Human AB Serum/1% (v/v) Penicillin/Streptomycin for 45 minutes. Overlapping peptide pools were plated at 4 μg/ml, 50 μL per well, DMSO (Sigma) was used as the negative control at the equivalent concentration to the peptides. 200,000 cells in 50 μl were added and incubated for 18-24 hours. Cells were discarded, plates washed with PBS 0.05% (v/v) Tween (Sigma), and incubated with IFNγ detector antibody (clone 7-B6-1, 1 ug/ml) for 2-4 hours at room temperature. Washed plates were then incubated with streptavidin alkaline phosphatase antibody (1 ug/ml) for 1-2 hours. Plates were washed then colour development was carried out using 1-step NBT/BCIP Substrate Solution. 50 μl of filtered NBT/BCIP was added to each well for 5 minutes at RT after which development was stopped with cold water. Plates were dried at room temperature for approximately 48 hours. Spots were quantified using an AID iSpot Spectrum EliSpot Reader (software version 7.0, Autoimmun Diagnostika, Strassberg). Average spot count value in the background wells was subtracted from the test wells and values expressed as spot forming units (SFU) per million cells. Mouse IFNγ ELISpot assays were performed using cryopreserved splenocytes thawed as above and incubated in RPMI1640 medium/10% FBS only. Peptide stimulations and downstream processing were as above, using pre-coated Mouse IFN-γ ELISpot PLUS kit (ALP) (MabTech 3321-4APT-2).

### Data Availability Statement

Mass spectrometry data are provided in Table 1 and deposited to the ProteomeXchange Consortium via the PRIDE partner repository ⁴⁰ (Accession PXD039483). Reviewer log-in details are username: reviewer_pxd039483@ebi.ac.uk and password: JAQVmZq3. RNA-seq reads and processed files are available at NCBI Gene Expression Omnibus (Accession GSE223044). Reviewer token: ajqbgkiavtghlkt. Additional data are available from the corresponding author upon reasonable request.

Mass spectrometry data have been deposited with MassIVE ID MSV000093074. RNA-seq reads and processed files are available at the NCBI Gene Expression Omnibus (accession GSE223044). Additional data are available from figshare (https://doi.org/10.6084/m9.figshare.24271744). The following accessions were used for mass spectrometry analysis: UP000001811 and P08659 (UniProt). Source data are provided in https://www.nature.com/articles/s41586-023-06800-3 #Sec23.

### Code Availability Statement

Scripts for processing alignments are available from GitHub 41.

### Methods References

Svitkin Y V, Sonenberg N. An efficient system for cap- and poly(A)-dependent translation in vitro., In: Schoenberg, D.R. (eds) mRNA Processing and Metabolism. Methods in Molecular Biology. Humana Press (Totowa). 257, 155-70 (2004). https://doi.org/10.1385/1-59259-750-5:155
Rosenfeld J, Capdevielle J, Guillemot JC, Ferrara P. In-gel digestion of proteins for internal sequence analysis after one- or two-dimensional gel electrophoresis. Anal Biochem. 203, 173-9 (1992). https://doi.org/10.1016/0003-2697 (92) 90061-b
Dobin A, et al. STAR: ultrafast universal RNA-seq aligner. Bioinformatics. 29, 15-21 (2013). https://doi.org/10.1093/bioinformatics/bts635
Payne R P, et al. Immunogenicity of standard and extended dosing intervals of BNT162b2 mRNA vaccine. Cell. 184, 5699-5714 (2021). https://doi.org/10.1016/j.cell.2021.10.011
Vizcaíno J A, et al. 2016 update of the PRIDE database and its related tools. Nucleic Acids Res. 44, D447-56 (2016). https://doi.org/10.1093/nar/gkv1145
Mulroney, T. E. RNA-seq_mutations. Available at: https://github.com/tom-mulroney/rna-seq_mutations. (Accessed: 23rd January 2023)

### Results & Discussion

A key feature of therapeutic IVTmRNAs is that they contain modified ribonucleotides, which have been shown to decrease innate immunogenicity and can additionally increase mRNA stability, both of which are favourable characteristics for therapies ^{1,2}. For example, clinically approved SARS-COV-2 mRNA vaccines incorporate (N)1-methylpseudouridine (1-methylΨ), which has been shown to decrease IVTmRNA innate immunogenicity ³⁻⁵. Some modified ribonucleotides, such as 5-methylcytidine (5-methylC), are naturally occurring post-transcriptional mRNA modifications in eukaryotes, while others are not, such as 1-methylΨ⁶⁻¹⁰.

How 5-methoxyuridine (5-methoxyU), 5-methylC, and 1-methyl affect translation of IVTmRNA was investigated. 5-methoxyU, 5-methylC, 1-methylΨ′ have been utilised in IVTmRNAs to attempt to increase recombinant protein synthesis in vitro, and for preclinical proof-of-concept for IVTmRNA-based therapies ^{11,12}. As mentioned, 1-methylΨ′ is a ribonucleotide incorporated in licensed IVTmRNA-based SARS-COV2 vaccines, but also mRNA-based human vaccines and therapies in development ^{4,13,14}

Despite widespread use, surprisingly little is known about how ribonucleotide modification affects protein synthesis, particularly for translation of therapeutic IVTmRNAs. This example investigates how modified ribonucleotides affect the fidelity of mRNA translation for several reasons. Certain ribonucleotide modifications can recode mRNA sequences, for example inosine ¹⁵. 5-methylC has previously been shown to increase misreading during mRNA translation in prokaryotes, but its effect on eukaryotic mRNA translation fidelity has not been explored ¹⁶. The effect of 5-methoxyU on translation fidelity has not been investigated. Pseudouridine (Ψ) is known to increase misreading of mRNA stop codons in eukaryotes, and can affect misreading during prokaryotic mRNA translation ¹⁶⁻¹⁸ 1-methylΨ does not appear to affect codon misreading, but has been shown to affect protein synthesis rates and ribosome density on mRNAs, suggesting a direct effect on mRNA translation ^{19,20}.

It is currently unclear which modified ribonucleotides affect mRNA translation fidelity and existing studies are mostly limited to understanding misreading frequencies at a given codon only. Misreading of mRNA codons is also only one type of posttranscriptional mechanism that can alter a polypeptide sequence. To date, no study has investigated the fundamental question of whether modified ribonucleotides can affect the maintenance of correct reading frame during translation of a synthetic transcript. Understanding these processes is critical to increase knowledge of protein synthesis of modified mRNAs in general, but is also imperative for the robust design and evaluation of novel mRNA-based therapeutics that make use of modified ribonucleotides within widely differing RNA sequences or therapeutic contexts.

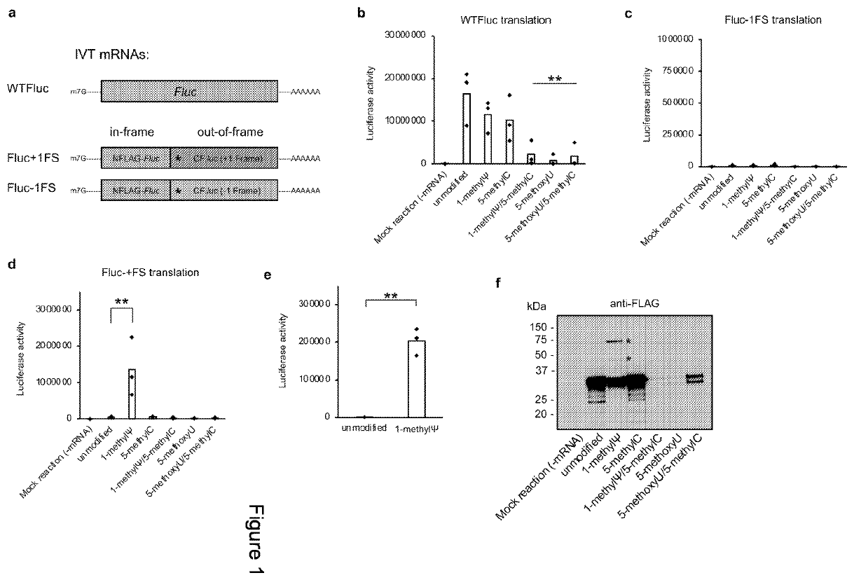

To investigate how ribonucleotide modification affects reading frame maintenance during translation of mRNA, we designed and synthesised IVTmRNAs which report on out-of-frame protein synthesis (FIG. 1a). These mRNAs encode an N-terminal segment of firefly luciferase (NFluc) and a complementary C-terminal segment of firefly luciferase (CFluc), directly downstream. CFluc is encoded in the −1 reading frame in Fluc-1FS, and in the +1 reading frame in Fluc +1FS. Fluc-1FS and Fluc +1FS mRNAs are designed to produce catalytically inactive (truncated) NFluc when translated normally. However, if ribosomes move out-of-frame during translation, elongated polypeptides containing residues from both in-frame NFluc and out-of-frame CFluc can be produced, which have increased catalytic activity.

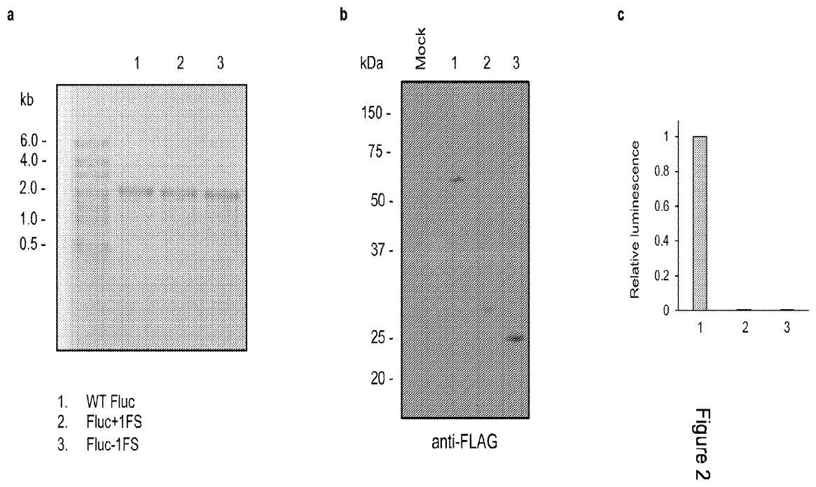

Unmodified Fluc-1FS and Fluc +1FS mRNAs was synthesised, which contain canonical ribonucleotides, and translated them in vitro. It was confirmed that Fluc-1FS and Fluc +1FS mRNAs produce catalytically inactive NFluc (FIG. 2). By comparison, unmodified WTFluc mRNA, containing complete in-frame firefly luciferase coding sequence, produced the expected active protein (FIG. 2). Then, each mRNA containing 5-methoxyU, 5-methylC, 1-methyl, 5-methoxyU and 5-methylC, or 1-methyl and 5-methylC was synthesised and translated. Translation of WTFluc mRNA was not significantly affected by either 1-methylΨ or 5-methylC modifications alone, but was decreased by incorporating both ribonucleotides into a single transcript (FIG. 1b). 5-methoxyU incorporation alone, or combined with 5-methylC, significantly decreased translation of WTFluc mRNA (FIG. 1b). No ribonucleotide modification appeared to affect ribosomal-1 frameshifting (FIG. 1c). However, incorporation of 1-methylΨ significantly increased ribosomal +1 frameshifting (FIG. 1d). Hela cells transfected with 1-methylΨ Fluc +1FS mRNA recapitulated the results from in vitro translation (FIG. 1e). Based on these observations, we concluded that IVTmRNA containing 1-methylΨ or 5-methylC exhibit similar translation efficiency to unmodified mRNA, but 1-methylΨsignificantly increases ribosomal +1 frameshifting during mRNA translation.

It was intriguing to observe a large increase in ribosomal +1 frameshifting during translation of 1-methylΨ mRNA and it was reasoned that gaining better understanding of the translation products would complement the reporter assay data and help to explain how +1 frameshifted products originate. To address these aspects, the polypeptides produced during IVTmRNA translation were probed by western blotting. Translation of unmodified Fluc +1FS mRNA produced the expected in-frame truncated product, which was also true for 5-methylC mRNA (FIG. 1f). Translation of 1-methylΨ mRNA produced the expected in-frame product, but also produced two additional bands at higher molecular weight (FIG. 1f). It was proposed that these products were +1 frameshifted polypeptides. It was also confirmed that 1-methyl/5-methylC-, 5-methoxyU-, and 5-methoxyU/5-methylC-mRNAs were comparatively poor mRNA templates for protein synthesis (FIG. 1f).

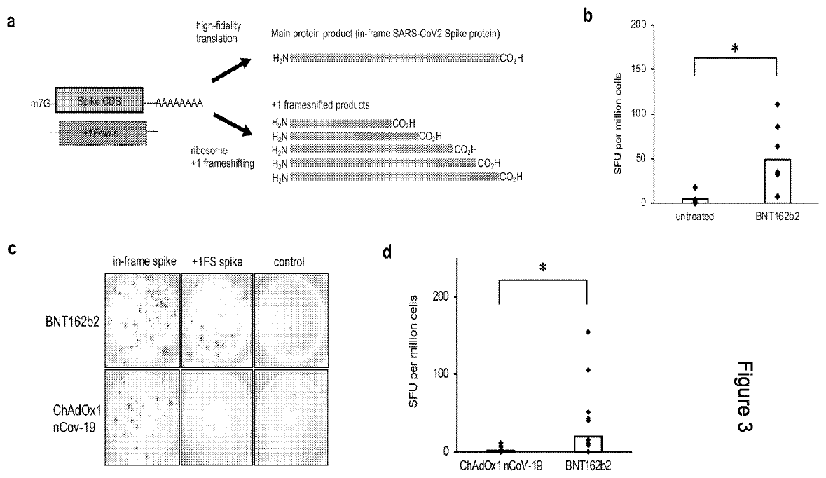

1-methylΨ is also used in clinically-approved SARS-COV2 mRNA vaccines 3,4. Since 1-methyl increased +1 ribosome frameshifting during translation in vitro, it was investigated whether this occurs in vivo for BNT162b2, a SARS-COV2 mRNA vaccine containing 1-methylΨ. It was reasoned that +1 ribosomal frameshifting during recombinant antigen mRNA translation could lead to presentation of +1 frameshifted products to T cells, and elicit off-target cellular immune responses (FIG. 3a). Antigen presentation from mistranslation of endogenous tumour mRNA has been shown to occur in vivo, for example 21 To address this possibility, mice were vaccinated with BNT162b2 and quantified their T cell response to in-frame SARS-COV2 spike protein and predicted +1 frameshifted products by interferon-gamma ELISpot assay. Responses to +1 frameshifted spike peptides were significantly increased in vaccinated mice compared to untreated mice (FIG. 3b). These data suggest that +1 frameshifted products encoded in BNT162b2 spike mRNA are T cell antigens for inbred mice, to which off-target immunity can be detected following vaccination.

Interferon-gamma ELISpot responses were compared to predicted +1 frameshifted SARS-CoV2 spike protein products in twenty-two individuals vaccinated with BNT162b2 and compared these responses to nineteen individuals vaccinated with ChAdOx1 nCOV-19, another SARS-COV2 vaccine that shares the same Spike protein antigen as BNT162b2, but which is not translated from 1-methylΨ mRNA 22. A significantly higher interferon-gamma response to +1 frameshifted antigen in the BNT162b2 vaccine group was detected, compared to ChAdOx1 nCOV-19 (FIG. 3C). During SARS-COV2 viral replication, a programmed −1 ribosomal frameshift occurs naturally during translation of ORF1a/b ²³. It is not feasible that these data are a consequence of natural SARS-COV2 infection for the following, non-exhaustive reasons. First, no frameshifting activity is known to occur during SARS-COV2 Spike subgenomic mRNA translation (which would be a major discovery in its own right). Secondly, −1 frameshifting (and not +1 frameshifting) is restricted to a single programmed site in ORF1a/b 23. Thirdly, +1 frameshifted peptides are predicted from the BNT162b2 mRNA sequence, and not the S gene sequence from wild virus. Instead, these data suggest that human vaccination with 1-methylΨ mRNA can elicit cellular immunity to peptide antigens produced by +1 ribosomal frameshifting.

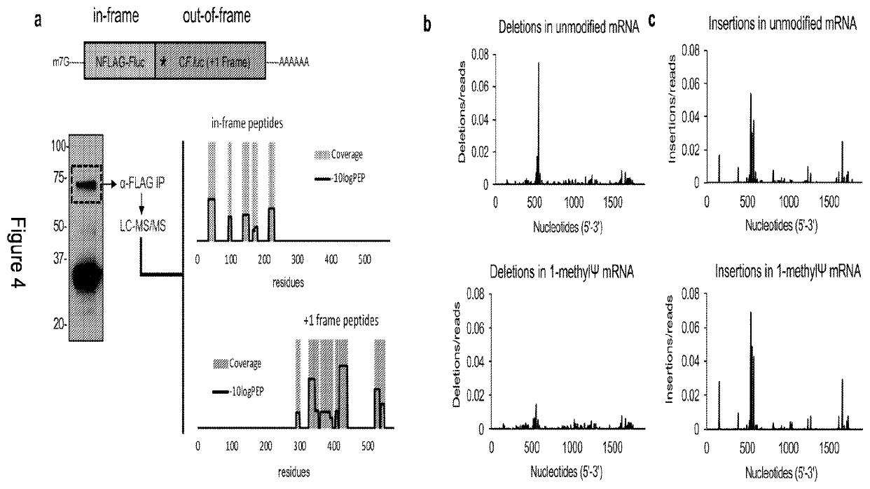

To provide further mechanistic insight into +1 ribosome frameshifting during translation of 1-methylΨ mRNA, and identify potential frameshift sites/sequences, a large-scale in vitro translation of 1-methylΨ Fluc +1FS mRNA was performed, the major putative +1 frameshifted polypeptide was purified, and liquid chromatography tandem mass spectrometry was carried out. From this single polypeptide six in-frame peptides and nine peptides derived from the mRNA +1 frame were identified (FIG. 4a and Table 1). All in-frame peptides were mapped to the N-terminal region, while +1 frameshifted peptides were mapped downstream (FIG. 4a). These data demonstrated that the elongated polypeptide was indeed a chimeric polypeptide consisting of in-frame N-terminal residues and +1 frameshifted C-terminal residues.

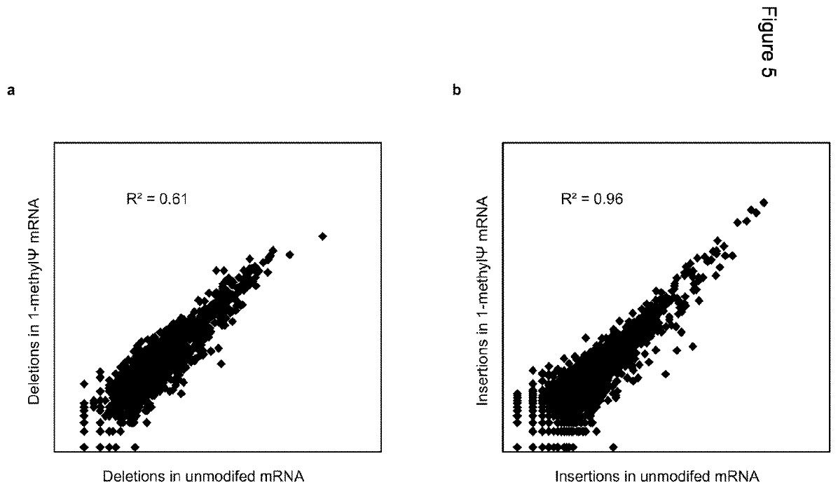

Errors in protein synthesis, including frameshifting, can be consequences of DNA mutation or transcriptional errors 24. Hence, faithful translation of an incorrect mRNA sequence can produce incorrect proteins. In vitro transcripts are presumed to be exact RNA copies of template DNA, the accuracy of which may be estimated by the fidelity of the employed RNA polymerase. However, the substitution of canonical substrate rNTPs for modified nucleotides may increase transcriptional errors. To address this possibility, high-throughput RNA sequencing of unmodified and 1-methylΨ′ Fluc +1FS mRNA was performed and nucleotide insertions and deletions in each population of IVTmRNA were quantified. Nucleotide deletion profiles for each mRNA were very similar (FIGS. 4b and 5), as were nucleotide insertions (FIG. 4c), suggesting few site-specific differences. The overall frequency of insertions and deletions was low, and did not differ significantly between unmodified and 1-methylΨ mRNA (Table 2), which is supported by recent observations 25. Taken together, it was concluded that frameshifted products of 1-methylΨ mRNA translation were not due to transcriptional errors, but were due to bona fide ribosomal +1 frameshifting—a posttranscriptional mechanism.

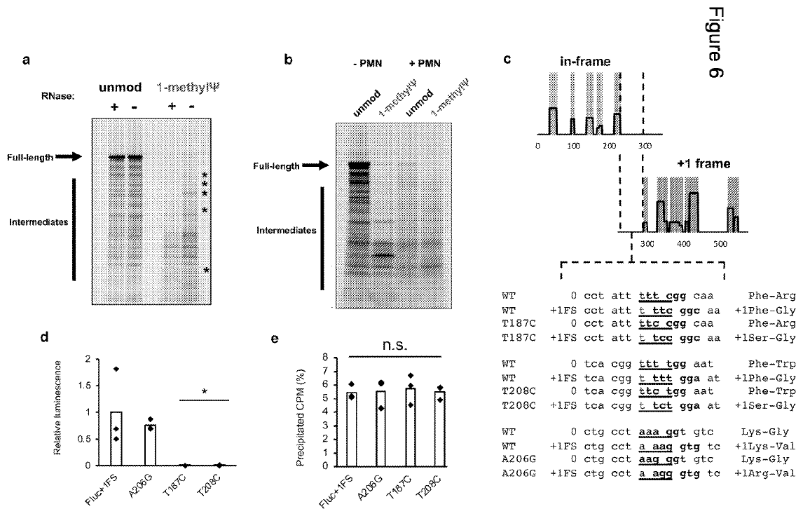

Ribosome frameshifting is a well-documented phenomenon that occurs during translation of many naturally occurring mRNAs 24. Ribosome stalling is implicated in several such mechanisms, and it was queried whether the presence of 1-methylΨ in IVTmRNA leads to ribosome stalling during translation 26-29. To do this, clearly intermediate peptidyl-tRNAs produced during translation of unmodified or 1-methylΨ WT Fluc mRNA were assayed, which are a consequence of ribosome stalling 30. While translation of unmodified mRNA produced no clearly observable clearly peptidyl-tRNAs, during translation of 1-methylΨ mRNA several stable peptidyl-tRNA intermediates were detected (FIG. 6a). Translation elongation of 1-methylΨ mRNA was also slower than for unmodified mRNA (FIG. 4a), which is supported by previous observations 20. All reactions were run for 30 minutes and there was less full-length protein produced from the translation of 1-methylΨ containing mRNAs, suggesting a slower elongation rate compared to unmodified mRNA, with a greater proportion of premature polypeptide products. These data strongly suggest that elongating ribosomes stall during translation of mRNA containing 1-methylΨ.

It was unclear whether 1-methylΨ affected mRNA decoding rates, or another process during elongation. It was reasoned that slower decoding of 1-methylΨ codons during translation elongation could lead to ribosome stalling, similar to previous observations for ‘hungry’ codons at sites of +1 frameshifting during translation of naturally occurring mRNA 21,28. The molecular mechanism of ribosome stalling during 1-methylΨ mRNA translation was probed using the aminoglycoside paromomycin. Very briefly, during mRNA decoding, cognate aminoacyl-tRNA anticodon:codon interaction causes local conformational changes in 18S rRNA (in eukaryotes), after which a new peptide bond is formed, ribosome subunit rotation occurs, and subsequent ribosome conformational changes, elongation factor 2 binding and translocation to the next codon completes the elongation cycle 31. Paromomycin binds to Helix 44 of 18S rRNA in elongating ribosomes and alters its conformation in the decoding centre, which inhibits translation but also permits the productive binding of near- and non-cognate aminoacyl-tRNAs to the 80S ribosome A-site 32. In doing so, paromomycin increases the misincorporation of amino acids into elongating polypeptides 33. It was hypothesised that ribosome stalling during 1-methylΨ mRNA translation could be decreased by paromomycin if slow decoding were due to altered aminoacyl-tRNA binding kinetics, as paromomycin-bound ribosomes could incorporate additional near- or non-cognate aminoacyl-tRNAs-effectively increasing the substrate aminoacyl-tRNA pool at stall sites. Translation of 1-methylΨ mRNA was slower than unmodified mRNA and the proportion of premature polypeptide products greater (FIGS. 6a and 6b). However, during 1-methyl mRNA translation, polypeptide elongation was actually improved by the addition of paromomycin, whereas paromomycin was only inhibitory to unmodified mRNA translation (FIG. 6b). Together, these data show that translation of 1-methylΨ mRNA is prone to ribosome stalling, likely caused by altered aminoacyl-tRNA binding, which can be rescued by increasing the incorporation of near- or non-cognate amino acids into elongating polypeptides.

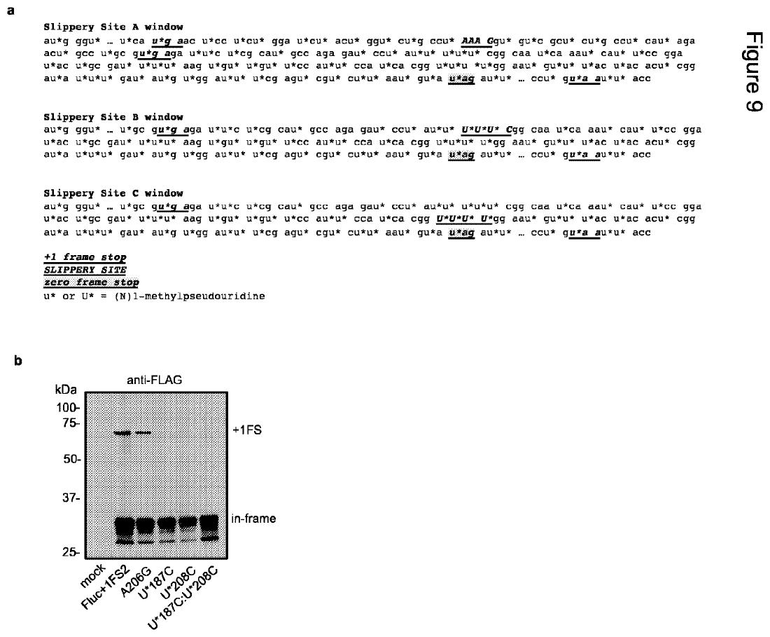

While there is no evidence that frameshifted products in humans generated from BNT162b2 vaccination (FIG. 3) are associated with adverse outcomes, for future use of mRNA technology it is important that mRNA sequence design is modified to reduced ribosome frameshifting events, as this may limit its future use for applications that require frequent dosing, such as the in vivo production of hormones. In such cases, the main in-frame product is unlikely to elicit an adaptive immune response, but presentation of +1 frameshifted products could activate T cells that target host cells expressing the recombinant hormone. It was reasoned that if able to identify +1 ribosome frameshift site/sequences it would be possible to alter the sequence to reduce such effects. As proof-of-principle a reporter IVTmRNA system was used. LC-MS/MS analysis showed that translation of 1-methylΨ mRNA leads to synthesis of +1 frameshifted products within the area of coding sequence between detected in-frame residues and downstream +1 frameshifted residues (FIG. 4a). The RNA sequence corresponding to this region was searched for determinants of ribosome frameshifting based on published mechanisms, from which three potential ribosome slippery sequences were identified (FIG. 6c), with all three sequences having the potential to be decoded by the same aminoacyl-tRNA at an in-frame codon or in the immediate +1 frame codon. It was hypothesised that these could therefore function as sites for +1 ribosomal frameshifting (FIG. 6c). These sites in 1-methylΨ Fluc +1FS mRNA were synonymously mutated such that the in-frame amino acid was unchanged, but the immediate +1 frame codon was mutated to a non-cognate amino acid, hence destroying the ribosome slippery sequence, and translated the therapeutic mRNAs to evaluate the contribution of each site to +1 ribosomal frameshifting (FIG. 6c). Luciferase activity produced by translation of A206G mRNA (Slippery Site A mutant) was no different to control levels, demonstrating that this site does not affect +1 ribosomal frameshifting (FIG. 6d). However, both T187C mRNA and T208C mRNA (Slippery Site B and C mutants, respectively) strongly decreased +1 ribosome frameshifting (FIG. 6d). Importantly, translation efficiency of each mRNA was equal, which suggested that no mutation adversely affected mRNA translation overall, but solely +1 ribosomal frameshifting activity (FIG. 6e). Translation of an mRNA containing both T187C and T208C mutations (mutation of both Slippery Site B and C) produced no detectable frameshifting activity (FIG. 9). Taken together, these data suggest that (N)1-methylpseudouridylation at defined mRNA sequences triggers ribosome +1 frameshifting, however, with appropriate mRNA sequence design it is possible to ameliorate this issue.

Mouse and human vaccination with BNT162b2 mRNA had led to increased cellular immunogenicity to +1 frame peptides (FIG. 3). However, it was unclear which ribosome slippery sites in the BNT162b2 RNA sequence contributed to +1 ribosomal frameshifting during BNT162b2 mRNA translation, and how local RNA sequence context affected the efficiency of ribosomal +1 frameshifting. We identified six ribosome slippery sites in the BNT162b2 RNA sequence (Slippery Sites 1-6), and another element which we speculated could support ribosome +1 frameshifting under certain conditions (Sequence X). The core motifs of Slippery Sites 1-6 were identical to Slippery Site B or Slippery Site C in Fluc +1FS mRNA, but (as expected) differed in their local RNA sequence context.

We individually assayed BNT162b2 Slippery Sites 1-6 and Sequence X for +1 ribosome frameshifting using a series of dual tag reporter mRNAs (SpikeFS1-SpikeFS6 mRNAs and SpikeSX mRNAs). Each mRNA coding sequence was constructed of an N-terminal 3xMyc-tag which was in-frame to an approximately 500-900 nucleotide-long section of the BNT162b2 mRNA coding sequence containing the relevant ribosome slippery site or Sequence X (FIG. 10a). A 3xFLAG-tag was inserted 10-25 nucleotides downstream of each ribosome slippery site or Sequence X, encoded in the mRNA +1 frame (FIG. 10a). Normal translation of each mRNA would be expected to produce a polypeptide consisting of N-terminal 3xMyc tag and a section of SARS-COV-2 Spike protein. However, ribosome +1 frameshifting at the upstream site would be expected to produce a chimeric polypeptide containing N-terminal 3xMyc-tag, a section of SARS-COV-2 Spike protein, and a short +1 frame mutation, including a C-terminal 3xFLAG. Candidate ribosome slippery sites for each mRNA are listed in FIG. 10b.

We synthesised the above mRNAs containing either canonical ribonucleotides (unmodified), or 1-methylΨ, and assayed their translation products by western blotting. By assaying abundance of anti-FLAG reactive products, we confidently identified +1 frame products from translation of 1-methylpseudouridylated SpikeFS1, SpikeSX, SpikeFS2, SpikeFS3, and SpikeFS6 mRNAs (FIG. 10c). These +1 frame products were not detected from translation of the unmodified mRNA equivalents. Interestingly, translation of 1-methylΨ′ SpikeFS4 and SpikeFS5 did not produce significant levels of +1 frame product.

We reasoned that synonymous mutation of the BNT162b2 ribosome slippery sequences could be used to decrease or abrogate +1 ribosomal frameshifting, as previously demonstrated by synonymous mutation of Slippery Site B and Slippery Site C in Fluc +1FS mRNA. To investigate this possibility, we synonymously mutated the candidate ribosome slippery sites of 1-methylpseudouridylated SpikeFS1, SpikeFS2, SpikeFS3, SpikeFS6, and SpikeSX, such that the decoding aminoacyl-tRNA of immediate +1 frame codon was no longer cognate for the main frame codon-destroying the ribosome slippery site, but without changing the amino acid sequence of the polypeptide encoded in the mRNA main frame—as previously performed for T187C and T208C (U*187C and U*208C) mRNAs. These mRNAs (AntiFS1, AntiFS2, AntiFS3, AntiFS6, and AntiSX) were then translated and assayed for in-frame and +1 frame translation as prior. As previously shown, +1 frame products were detected by translation of 1-methylΨ SpikeFS1, SpikeFS2, SpikeFS3, SpikeFS6, and SpikeSX mRNAs (FIG. 10d). However, translation of 1-methylΨ AntiFS1, AntiFS2, AntiFS3, AntiFS6, and AntiSX mRNAs led to significantly decreased synthesis of +1 frame products, while supporting in-frame mRNA translation (FIG. 10d). Taken together, these data show that it is possible to search an mRNA sequence for potential sites of ribosomal +1 frameshifting, that ribosomal +1 frameshifting occurs during translation of the majority of mRNAs containing these predicted sites, and that targeted mutation of these sites can significantly decrease ribosomal +1 frameshifting during mRNA translation.

### CONCLUSIONS

It is shown that 1-methylΨ is a modified ribonucleotide that significantly increases +1 ribosomal frameshifting during mRNA translation and that cellular immunity to +1 frameshifted products can occur following vaccination with mRNA containing 1-methylΨ. To our knowledge, this is the first report that mRNA modification affects ribosomal frameshifting. Other ribonucleotide modification strategies, such as incorporation of 5-methoxyU, significantly decreases translation efficiency of IVTmRNAs, which may limit clinical translation. It is shown that IVTmRNAs contain few nucleotide insertions/deletions, and this is not changed by 1-methylΨ incorporation. +1 ribosomal frameshifting during translation of 1-methylΨ mRNA is affected by ribosome slippery sequences. Translation of mRNA containing 1-methylΨ leads to ribosome stalling. Stalling is most likely caused by altered aminoacyl-tRNA binding, which demonstrates why +1 ribosomal frameshifting may not occur during unmodified mRNA translation—both ribosome stalling and ribosome slippery sequences are important to productive +1 ribosome frameshifting. The mechanistic data presented herein are supported by previous observations of ribosomal frameshifting during translation of naturally occurring mRNAs, which implicate ribosome stalling and ribosome slippery sequences for +1 frameshifting 21,26-29,34,35. We also show that targeted mutation of ribosome slippery sites can decrease +1 ribosomal frameshifting, but retain in-frame mRNA translation. These novel findings are of particular importance to the fundamental understanding of how ribonucleotide modification affects mRNA translation, and for designing and optimising future mRNA-based therapeutics to avoid mistranslation events that may decrease efficacy and/or increase toxicity.

### Example 2

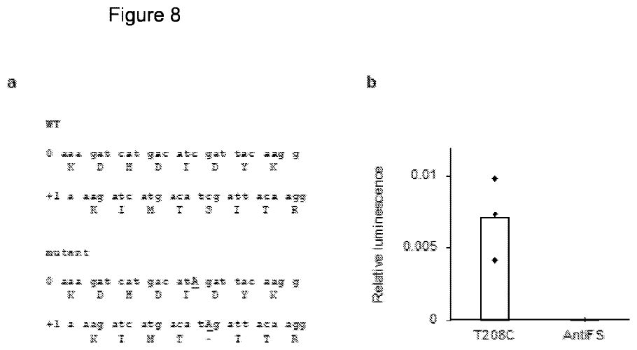

A second process to reduce out-of-frame translation events was to identify and remove other sequences which otherwise support out-of-frame protein synthesis while preserving the encoded protein product. This was achieved by disrupting alternative reading frames with premature termination codons (PTCs) within those reading frames. The protein coding sequence was designed such that PTCs are not encountered unless out-of-frame protein synthesis is initiated. PTCs prevented peptide elongation in alternative reading frames by terminating translation, and reduced levels of +1 ribosome frameshifting to non-detectable levels (FIG. 8). Synthesis and presentation of out-of-frame peptides is a source of off-target cellular immunity (FIG. 3), and a potential source of toxicity in future mRNA-based drugs. PTCs may additionally act to destabilise mRNAs undergoing out-of-frame protein synthesis.

### Methods

T208C (SEQ ID NO: 8) and AntiFS (SEQ ID NO: 9) were transcribed using TranscriptAid T7 High Yield Transcription kit (Thermo Scientific), 5′-capped using Vaccinia Capping System (NEB M2080S), and purified. Templates were produced by BamHI digest of custom plasmids (Genscript Biotech Corporation). The gene sequences of T208C and AntiFS are provided below. To assay +1 ribosome frameshifting, 50 nM IVT mRNAs were translated in nuclease-treated RRL (Promega), described previously [13]. Luciferase activity after 2 hours was assayed using the Luciferase Assay System (Promega).

### Example 2 References

[1] Andries O, Mc Cafferty S, De Smedt SC, Weiss R, Sanders NN, Kitada T. N (1)-methylpseudouridine-incorporated mRNA outperforms pseudouridine-incorporated mRNA by providing enhanced protein expression and reduced immunogenicity in mammalian cell lines and mice. J Control Release 217, 337-44. doi: 10.1016/j.jconrel.2015.08.051 (2015).
[2] Li, B., Luo, X. & Dong, Y. Effects of Chemically Modified Messenger RNA on Protein Expression. Bioconjugate Chemistry 27, 849-853, doi: 10.1021/acs.bioconjchem.6b00090 (2016).
[3] Zangi, L. et al. Modified mRNA directs the fate of heart progenitor cells and induces vascular regeneration after myocardial infarction. Nat Biotechnol 31, 898-907, doi: 10.1038/nbt.2682 (2013).
[4] Stadler, C. R. et al. Elimination of large tumors in mice by mRNA-encoded bispecific antibodies. Nat Med 23, 815-817, doi: 10.1038/nm.4356 (2017).
[5] Pardi, N. et al. Nucleoside-modified mRNA immunization elicits influenza virus hemagglutinin stalk-specific antibodies. Nat Commun 9, 3361, doi: 10.1038/s41467-018-05482-0 (2018)
[6] Hogan, M. J. & Pardi, N. mRNA Vaccines in the COVID-19 Pandemic and Beyond. Annu Rev Med 73, 17-39, doi: 10.1146/annurev-med-042420-112725 (2022).
[7] Chaudhary, N., Weissman, D. & Whitehead, K. A. mRNA vaccines for infectious diseases: principles, delivery and clinical translation. Nat Rev Drug Discov 20, 817-838, doi: 10.1038/s41573-021-00283-5 (2021).
[8] Hoernes T P, Clementi N, Faserl K, Glasner H, Breuker K, Lindner H, Hüttenhofer A, Erlacher M D. Nucleotide modifications within bacterial messenger RNAs regulate their translation and are able to rewire the genetic code. Nucleic Acids Res. 44, 852-62. doi: 10.1093/nar/gkv1182 (2016).
[9] Licht K, Hartl M, Amman F, Anrather D, Janisiw M P, Jantsch M F. Inosine induces context-dependent recoding and translational stalling. Nucleic Acids Res. 47, 3-14. doi: 10.1093/nar/gkyl163 (2019)
[10] Svitkin Y V, Cheng Y M, Chakraborty T, Presnyak V, John M, Sonenberg N. N1-methyl-pseudouridine in mRNA enhances translation through elF2α-dependent and independent mechanisms by increasing ribosome density. Nucleic Acids Res. 45, 6023-6036. doi: 10.1093/nar/gkx135 (2017).
[11] Svitkin YV, Gingras AC, Sonenberg N. Membrane-dependent relief of translation elongation arrest on pseudouridine- and N1-methyl-pseudouridine-modified mRNAs. Nucleic Acids Res. 50, 7202-7215. doi: 10.1093/nar/gkab1241 (2022).
[12] Karijolich J, Yu YT. Converting nonsense codons into sense codons by targeted pseudouridylation. Nature 474, 395-8. doi: 10.1038/nature10165 (2011).
[13] Svitkin YV, Sonenberg N. An efficient system for cap- and poly(A)-dependent translation in vitro., In: Schoenberg, D.R. (eds) mRNA Processing and Metabolism. Methods in Molecular Biology. Humana Press (Totowa). 257, 155-70 https://doi.org/10.1385/1-59259-750-5:155 (2004).

The reader's attention is directed to all papers and documents which are filed concurrently with or previous to this specification in connection with this application and which are open to public inspection with this specification, and the contents of all such papers and documents are incorporated herein by reference.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings), may be replaced by alternative features serving the same, equivalent, or similar purpose, unless expressly stated otherwise. Thus, unless expressly stated otherwise, each feature disclosed is one example only of a generic series of equivalent or similar features.

The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

### REFERENCES

1 Anderson, B. R. et al. Incorporation of pseudouridine into mRNA enhances translation by diminishing PKR activation. Nucleic Acids Res 38, 5884-5892, doi: 10.1093/nar/gkq347 (2010).
2 Holtkamp, S. et al. Modification of antigen-encoding RNA increases stability, translational efficacy, and T-cell stimulatory capacity of dendritic cells. Blood 108, 4009-4017, doi: 10.1182/blood-2006-04-015024 (2006).
3 Hogan, M. J. & Pardi, N. mRNA Vaccines in the COVID-19 Pandemic and Beyond. Annu Rev Med 73, 17-39, doi: 10.1146/annurev-med-042420-112725 (2022).
4 Chaudhary, N., Weissman, D. & Whitehead, K. A. mRNA vaccines for infectious diseases: principles, delivery and clinical translation. Nat Rev Drug Discov 20, 817-838, doi: 10.1038/s41573-021-00283-5 (2021).
5 Andries, O. et al. N (1)-methylpseudouridine-incorporated mRNA outperforms pseudouridine-incorporated mRNA by providing enhanced protein expression and reduced immunogenicity in mammalian cell lines and mice. J Control Release 217, 337-344, doi: 10.1016/j.jconrel.2015.08.051 (2015).
6 Boo, S. H. & Kim, Y. K. The emerging role of RNA modifications in the regulation of mRNA stability. Exp Mol Med 52, 400-408, doi: 10.1038/s12276-020-0407-z (2020).
7 Squires, J. E. et al. Widespread occurrence of 5-methylcytosine in human coding and non-coding RNA. Nucleic Acids Res 40, 5023-5033, doi: 10.1093/nar/gks144 (2012).
8 Argoudelis, A. D. & Mizsak, S. A. 1-methylpseudouridine, a metabolite of Streptomyces platensis. J Antibiot (Tokyo) 29, 818-823, doi: 10.7164/antibiotics.29.818 (1976).
9 Pang, H. et al. Structure of a modified nucleoside in archaebacterial tRNA which replaces ribosylthymine. 1-Methylpseudouridine. J Biol Chem 257, 3589-3592 (1982).
10 Brand, R. C., Klootwijk, J., Planta, R. J. & Maden, B. E. Biosynthesis of a hypermodified nucleotide in Saccharomyces carlsbergensis 17S and HeLa-cell 18S ribosomal ribonucleic acid. Biochem J 169, 71-77, doi: 10.1042/bj1690071 (1978).
11 Li, B., Luo, X. & Dong, Y. Effects of Chemically Modified Messenger RNA on Protein Expression. Bioconjugate Chemistry 27, 849-853, doi: 10.1021/acs.bioconjchem.6b00090 (2016).
12 Zangi, L. et al. Modified mRNA directs the fate of heart progenitor cells and induces vascular regeneration after myocardial infarction. Nat Biotechnol 31, 898-907, doi: 10.1038/nbt.2682 (2013).
13 Stadler, C. R. et al. Elimination of large tumors in mice by mRNA-encoded bispecific antibodies. Nat Med 23, 815-817, doi: 10.1038/nm.4356 (2017).
14 Pardi, N. et al. Nucleoside-modified mRNA immunization elicits influenza virus hemagglutinin stalk-specific antibodies. Nat Commun 9, 3361, doi: 10.1038/s41467-018-05482-0 (2018).
15 Licht, K. et al. Inosine induces context-dependent recoding and translational stalling. Nucleic Acids Res 47, 3-14, doi: 10.1093/nar/gkyl163 (2019).
16 Hoernes, T. P. et al. Nucleotide modifications within bacterial messenger RNAs regulate their translation and are able to rewire the genetic code. Nucleic Acids Res 44, 852-862, doi: 10.1093/nar/gkv1182 (2016).
17 Karijolich, J. & Yu, Y. T. Converting nonsense codons into sense codons by targeted pseudouridylation. Nature 474, 395-398, doi: 10.1038/nature10165 (2011).
18 Eyler, D. E. et al. Pseudouridinylation of mRNA coding sequences alters translation. Proc Natl Acad Sci USA 116, 23068-23074, doi: 10.1073/pnas. 1821754116 (2019).
19 Kim, K. Q. et al. N1-methylpseudouridine found within COVID-19 mRNA vaccines produces faithful protein products. Cell Rep 40 111300, doi: 10.1016/j.celrep.2022.111300 (2022).
20 Svitkin, Y. V. et al. N1-methyl-pseudouridine in mRNA enhances translation through elF2α-dependent and independent mechanisms by increasing ribosome density. Nucleic Acids Res 45, 6023-6036, doi: 10.1093/nar/gkx135 (2017).
21 Bartok, O. et al. Anti-tumour immunity induces aberrant peptide presentation in melanoma. Nature 590, 332-337, doi: 10.1038/s41586-020-03054-1 (2021).
22 Folegatti, P. M. et al. Safety and immunogenicity of the ChAdOx1 nCOV-19 vaccine against SARS-COV-2: a preliminary report of a phase 1/2, single-blind, randomised controlled trial. Lancet 396, 467-478, doi: 10.1016/s0140-6736 (20) 31604-4 (2020).
23 Bhatt, P. R. et al. Structural basis of ribosomal frameshifting during translation of the SARS-COV-2 RNA genome. Science 372, 1306-1313, doi: 10.1126/science.abf3546 (2021).
24 Ketteler, R. On programmed ribosomal frameshifting: the alternative proteomes. Front Genet 3, 242, doi: 10.3389/fgene.2012.00242 (2012).
25 Chen, T. H., Potapov, V., Dai, N., Ong, J. L. & Roy, B. N (1)-methyl-pseudouridine is incorporated with higher fidelity than pseudouridine in synthetic RNAs. Sci Rep 12, 13017, doi: 10.1038/s41598-022-17249-1 (2022).
26 Gamper, H. B., Masuda, I., Frenkel-Morgenstern, M. & Hou, Y. M. Maintenance of protein synthesis reading frame by EF-P and m (1) G37-tRNA. Nat Commun 6, 7226, doi: 10.1038/ncomms8226 (2015).
27 Qian, Q. et al. A new model for phenotypic suppression of frameshift mutations by mutant tRNAs. Mol Cell 1, 471-482, doi: 10.1016/s1097-2765 (00) 80048-9 (1998).
28 O'Connor, M. Imbalance of tRNA (Pro) isoacceptors induces +1 frameshifting at near-cognate codons. Nucleic Acids Res 30, 759-765, doi: 10.1093/nar/30.3.759 (2002).
29 Masuda, I. et al. Loss of N (1)-methylation of G37 in tRNA induces ribosome stalling and reprograms gene expression. Elife 10, doi: 10.7554/eLife.70619 (2021).
30 Stoneley, M. et al. Unresolved stalled ribosome complexes restrict cell-cycle progression after genotoxic stress. Mol Cell 82, 1557-1572.e1557, doi: 10.1016/j.molcel.2022.01.019 (2022).
31 Lareau, L. F., Hite, D. H., Hogan, G. J. & Brown, P. O. Distinct stages of the translation elongation cycle revealed by sequencing ribosome-protected mRNA fragments. Elife 3, e01257, doi: 10.7554/eLife.01257 (2014).
32 Prokhorova, I. et al. Aminoglycoside interactions and impacts on the eukaryotic ribosome. Proc Natl Acad Sci USA 114, E10899-e10908, doi: 10.1073/pnas. 1715501114 (2017).
33 Tuite, M. F. & Mclaughlin, C. S. The effects of paromomycin on the fidelity of translation in a yeast cell-free system. Biochim Biophys Acta 783, 166-170, doi: 10.1016/0167-4781 (84) 90009-5 (1984).
34 Jacks, T., Madhani, H. D., Masiarz, F. R. & Varmus, H. E. Signals for ribosomal frameshifting in the Rous sarcoma virus gag-pol region. Cell 55, 447-458, doi: 10.1016/0092-8674 (88) 90031-1 (1988).
35 Devaraj, A. & Fredrick, K. Short spacing between the Shine-Dalgarno sequence and P codon destabilizes codon-anticodon pairing in the P site to promote +1 programmed frameshifting. Mol Microbiol 78, 1500-1509, doi: 10.1111/j.1365-2958.2010.07421.x (2010).

### SEQUENCES

| | | SEQ |
|---|---|---|
| SEQUENCE | Description | ID NO |
| KDHDIDYK | encoded peptide of example 2 | 1 |
| UUU UGA | +1 frameshift sequence | 2 |
| UCCUGA | +1 frameshift sequence | 3 |
| CCCUGA | +1 frameshift sequence | 4 |
| X XXY YYZ | Slippery sequence consensus- | 5 |
| | wherein X is any nucleotide, wherein Y is A | |
| | or U, and wherein Z is A, U, or C | |
| GAGAAm1ΨAAACm1ΨAGm1ΨAm1Ψm1ΨCm1Ψ | BNT162b2 | 6 |
| m1ΨCm1ΨGGm1ΨCCCCACAGACm1ΨCAGAGA | | |
| GAACCCGCCACCAm1ΨGm1Ψm1ΨCGm1ΨGm1 | | |
| Ψm1ΨCCm1ΨGGm1ΨGCm1ΨGCm1ΨGCCm1ΨC | | |
| m1ΨGGm1ΨGm1ΨCCAGCCAGm1ΨGm1ΨGm1Ψ | | |
| GAACCm1ΨGACCACCAGAACACAGCm1ΨGCCm | | |
| 1ΨCCAGCCm1ΨACACCAACAGCm1Ψm1Ψm1ΨA | | |
| CCAGAGGCGm1ΨGm1ΨACm1ΨACCCCGACAA | | |
| GGm1ΨGm1Ψm1ΨCAGAm1ΨCCAGCGm1ΨGCm | | |
| 1ΨGCACm1ΨCm1ΨACCCAGGACCm1ΨGm1Ψm1 | | |
| ΨCCm1ΨGCCm1Ψm1Ψm1ΨCm1Ψm1ΨCAGCAA | | |
| CGm1ΨGACCm1ΨGGm1Ψm1ΨCCACGCCAm1Ψ | | |
| CCACGm1ΨGm1ΨCCGGCACCAAm1ΨGGCACC | | |
| AAGAGAm1Ψm1ΨCGACAACCCCGm1ΨGCm1Ψ | | |
| GCCCm1Ψm1ΨCAACGACGGGGm1ΨGm1ΨACm | | |
| 1Ψm1Ψm1ΨGCCAGCACCGAGAAGm1ΨCCAACA | | |
| m1ΨCAm1ΨCAGAGGCm1ΨGGAm1ΨCm1Ψm1Ψ | | |
| CGGCACCACACm1ΨGGACAGCAAGACCCAGAG | | |
| CCm1ΨGCm1ΨGAm1ΨCGm1ΨGAACAACGCCA | | |
| CCAACGm1ΨGGm1ΨCAm1ΨCAAAGm1ΨGm1Ψ | | |
| GCGAGm1Ψm1ΨCCAGm1Ψm1ΨCm1ΨGCAACG | | |
| ACCCCm1Ψm1ΨCCm1ΨGGGCGm1ΨCm1ΨACm | | |
| 1ΨACCACAAGAACAACAAGAGCm1ΨGGAm1ΨG | | |
| GAAAGCGAGm1Ψm1ΨCCGGGm1ΨGm1ΨACAG | | |
| CAGCGCCAACAACm1ΨGCACCm1Ψm1ΨCGAG | | |
| m1ΨACGm1ΨGm1ΨCCCAGCCm1Ψm1Ψm1ΨCC | | |
| m1ΨGAm1ΨGGACCm1ΨGGAAGGCAAGCAGGG | | |
| CAACm1Ψm1ΨCAAGAACCm1ΨGCGCGAGm1Ψ | | |
| m1ΨCGm1ΨGm1Ψm1Ψm1ΨAAGAACAm1ΨCGA | | |
| CGGCm1ΨACm1Ψm1ΨCAAGAm1ΨCm1ΨACAG | | |
| CAAGCACACCCCm1ΨAm1ΨCAACCm1ΨCGm1Ψ | | |
| GCGGGAm1ΨCm1ΨGCCm1ΨCAGGGCm1Ψm1Ψ | | |
| Cm1ΨCm1ΨGCm1ΨCm1ΨGGAACCCCm1ΨGGm | | |
| 1ΨGGAm1ΨCm1ΨGCCCAm1ΨCGGCAm1ΨCAA | | |
| CAm1ΨCACCCGGm1Ψm1Ψm1ΨCAGACACm1Ψ | | |
| GCm1ΨGGCCCm1ΨGCACAGAAGCm1ΨACCm1 | | |
| ΨGACACCm1ΨGGCGAm1ΨAGCAGCAGCGGAm | | |
| 1ΨGGACAGCm1ΨGGm1ΨGCCGCCGCm1Ψm1Ψ | | |
| ACm1ΨAm1ΨGm1ΨGGGCm1ΨACCm1ΨGCAGC | | |
| Cm1ΨAGAACCm1Ψm1ΨCCm1ΨGCm1ΨGAAGm | | |
| 1ΨACAACGAGAACGGCACCAm1ΨCACCGACGC | | |
| CGm1ΨGGAm1Ψm1ΨGm1ΨGCm1ΨCm1ΨGGAm | | |
| 1ΨCCm1ΨCm1ΨGAGCGAGACAAAGm1ΨGCACC | | |
| Cm1ΨGAAGm1ΨCCm1Ψm1ΨCACCGm1ΨGGAA | | |
| AAGGGCAm1ΨCm1ΨACCAGACCAGCAACm1Ψm | | |
| 1ΨCCGGGm1ΨGCAGCCCACCGAAm1ΨCCAm1 | | |
| ΨCGm1ΨGCGGm1Ψm1ΨCCCCAAm1ΨAm1ΨCA | | |
| CCAAm1ΨCm1ΨGm1ΨGCCCCm1Ψm1ΨCGGCG | | |
| AGGm1ΨGm1Ψm1ΨCAAm1ΨGCCACCAGAm1Ψ | | |
| m1ΨCGCCm1ΨCm1ΨGm1ΨGm1ΨACGCCm1ΨG | | |
| GAACCGGAAGCGGAm1ΨCAGCAAm1Ψm1ΨGC | | |
| Gm1ΨGGCCGACm1ΨACm1ΨCCGm1ΨGCm1ΨG | | |
| m1ΨACAACm1ΨCCGCCAGCm1Ψm1ΨCAGCACC | | |
| m1Ψm1ΨCAAGm1ΨGCm1ΨACGGCGm1ΨGm1Ψ | | |
| CCCCm1ΨACCAAGCm1ΨGAACGACCm1ΨGm1Ψ | | |
| GCm1Ψm1ΨCACAAACGm1ΨGm1ΨACGCCGACA | | |
| GCm1Ψm1ΨCGm1ΨGAm1ΨCCGGGGAGAm1ΨG | | |
| AAGm1ΨGCGGCAGAm1Ψm1ΨGCCCCm1ΨGGA | | |
| CAGACAGGCAAGAm1ΨCGCCGACm1ΨACAACm | | |
| 1ΨACAAGCm1ΨGCCCGACGACm1Ψm1ΨCACCG | | |
| GCm1ΨGm1ΨGm1ΨGAm1Ψm1ΨGCCm1ΨGGAA | | |
| CAGCAACAACCm1ΨGGACm1ΨCCAAAGm1ΨCG | | |
| GCGGCAACm1ΨACAAm1Ψm1ΨACCm1ΨGm1ΨA | | |
| CCGGCm1ΨGm1Ψm1ΨCCGGAAGm1ΨCCAAm1 | | |
| ΨCm1ΨGAAGCCCm1Ψm1ΨCGAGCGGGACAm1 | | |
| ΨCm1ΨCCACCGAGAm1ΨCm1ΨAm1ΨCAGGCC | | |
| GGCAGCACCCCm1Ψm1ΨGm1ΨAACGGCGm1Ψ | | |
| GGAAGGCm1Ψm1ΨCAACm1ΨGCm1ΨACm1Ψm | | |
| 1ΨCCCACm1ΨGCAGm1ΨCCm1ΨACGGCm1Ψm | | |
| 1Ψm1ΨCAGCCCACAAAm1ΨGGCGm1ΨGGGCm | | |
| 1ΨAm1ΨCAGCCCm1ΨACAGAGm1ΨGGm1ΨGG | | |
| m1ΨGCm1ΨGAGCm1Ψm1ΨCGAACm1ΨGCm1Ψ | | |
| GCAm1ΨGCCCCm1ΨGCCACAGm1ΨGm1ΨGCG | | |
| GCCCm1ΨAAGAAAAGCACCAAm1ΨCm1ΨCGm1 | | |
| ΨGAAGAACAAAm1ΨGCGm1ΨGAACm1Ψm1ΨCA | | |
| ACm1Ψm1ΨCAACGGCCm1ΨGACCGGCACCGG | | |
| CGm1ΨGCm1ΨGACAGAGAGCAACAAGAAGm1Ψ | | |
| m1ΨCCm1ΨGCCAm1Ψm1ΨCCAGCAGm1Ψm1Ψ | | |
| m1ΨGGCCGGGAm1ΨAm1ΨCGCCGAm1ΨACCA | | |
| CAGACGCCGm1Ψm1ΨAGAGAm1ΨCCCCAGACA | | |
| Cm1ΨGGAAAm1ΨCCm1ΨGGACAm1ΨCACCCC | | |
| m1Ψm1ΨGCAGCm1Ψm1ΨCGGCGGAGm1ΨGm1 | | |
| ΨCm1ΨGm1ΨGAm1ΨCACCCCm1ΨGGCACCAA | | |
| CACCAGCAAm1ΨCAGGm1ΨGGCAGm1ΨGCm1 | | |
| ΨGm1ΨACCAGGACGm1ΨGAACm1ΨGm1ΨACC | | |
| GAAGm1ΨGCCCGm1ΨGGCCAm1Ψm1ΨCACGC | | |
| CGAm1ΨCAGCm1ΨGACACCm1ΨACAm1ΨGGC | | |
| GGGm1ΨGm1ΨACm1ΨCCACCGGCAGCAAm1Ψ | | |
| Gm1ΨGm1Ψm1Ψm1ΨCAGACCAGAGCCGGCm1 | | |
| ΨGm1ΨCm1ΨGAm1ΨCGGAGCCGAGCACGm1Ψ | | |
| GAACAAm1ΨAGCm1ΨACGAGm1ΨGCGACAm1Ψ | | |
| CCCCAm1ΨCGGCGCm1ΨGGAAm1ΨCm1ΨGCG | | |
| CCAGCm1ΨACCAGACACAGACAAACAGCCCm1 | | |
| ΨCGGAGAGCCAGAAGCGm1ΨGGCCAGCCAGA | | |
| GCAm1ΨCAm1Ψm1ΨGCCm1ΨACACAAm1ΨGm1 | | |
| ΨCm1ΨCm1ΨGGGCGCCGAGAACAGCGm1ΨGG | | |
| CCm1ΨACm1ΨCCAACAACm1ΨCm1ΨAm1ΨCGC | | |
| m1ΨAm1ΨCCCCACCAACm1Ψm1ΨCACCAm1ΨC | | |
| AGCGm1ΨGACCACAGAGAm1ΨCCm1ΨGCCm1 | | |
| ΨGm1ΨGm1ΨCCAm1ΨGACCAAGACCAGCGm1 | | |
| ΨGGACm1ΨGCACCAm1ΨGm1ΨACAm1ΨCm1Ψ | | |
| GCGGCGAm1Ψm1ΨCCACCGAGm1ΨGCm1ΨCC | | |
| AACCm1ΨGCm1ΨGCm1ΨGCAGm1ΨACGGCAG | | |
| Cm1Ψm1ΨCm1ΨGCACCCAGCm1ΨGAAm1ΨAG | | |
| AGCCCm1ΨGACAGGGAm1ΨCGCCGm1ΨGGAA | | |
| CAGGACAAGAACACCCAAGAGGm1ΨGm1Ψm1Ψ | | |
| CGCCCAAGm1ΨGAAGCAGAm1ΨCm1ΨACAAGA | | |
| CCCCm1ΨCCm1ΨAm1ΨCAAGGACm1Ψm1ΨCG | | |
| GCGGCm1Ψm1ΨCAAm1Ψm1Ψm1ΨCAGCCAGA | | |
| m1Ψm1ΨCm1ΨGCCCGAm1ΨCCm1ΨAGCAAGC | | |
| CCAGCAAGCGGAGCm1Ψm1ΨCAm1ΨCGAGGA | | |
| CCm1ΨGCm1ΨGm1Ψm1ΨCAACAAAGm1ΨGACA | | |
| Cm1ΨGGCCGACGCCGGCm1Ψm1ΨCAm1ΨCAA | | |
| GCAGm1ΨAm1ΨGGCGAm1Ψm1ΨGm1ΨCm1ΨG | | |
| GGCGACAm1Ψm1ΨGCCGCCAGGGAm1ΨCm1Ψ | | |
| GAm1Ψm1Ψm1ΨGCGCCCAGAAGm1Ψm1Ψm1Ψ | | |
| AACGGACm1ΨGACAGm1ΨGCm1ΨGCCm1ΨCC | | |
| m1ΨCm1ΨGCm1ΨGACCGAm1ΨGAGAm1ΨGAm | | |
| 1ΨCGCCCAGm1ΨACACAm1ΨCm1ΨGCCCm1Ψ | | |
| GCm1ΨGGCCGGCACAAm1ΨCACAAGCGGCm1 | | |
| ΨGGACAm1Ψm1Ψm1ΨGGAGCAGGCGCCGCm1 | | |
| ΨCm1ΨGCAGAm1ΨCCCCm1Ψm1Ψm1ΨGCm1Ψ | | |
| Am1ΨGCAGAm1ΨGGCCm1ΨACCGGm1Ψm1ΨC | | |
| AACGGCAm1ΨCGGAGm1ΨGACCCAGAAm1ΨG | | |
| m1ΨGCm1ΨGm1ΨACGAGAACCAGAAGCm1ΨG | | |
| Am1ΨCGCCAACCAGm1Ψm1ΨCAACAGCGCCAm | | |
| 1ΨCGGCAAGAm1ΨCCAGGACAGCCm1ΨGAGCA | | |
| GCACAGCAAGCGCCCm1ΨGGGAAAGCm1ΨGC | | |
| AGGACGm1ΨGGm1ΨCAACCAGAAm1ΨGCCCA | | |
| GGCACm1ΨGAACACCCm1ΨGGm1ΨCAAGCAG | | |
| Cm1ΨGm1ΨCCm1ΨCCAACm1Ψm1ΨCGGCGCC | | |
| Am1ΨCAGCm1ΨCm1ΨGm1ΨGCm1ΨGAACGAm | | |
| 1ΨAm1ΨCCm1ΨGAGCAGACm1ΨGGACCCm1Ψ | | |
| CCm1ΨGAGGCCGAGGm1ΨGCAGAm1ΨCGACA | | |
| GACm1ΨGAm1ΨCACAGGCAGACm1ΨGCAGAG | | |
| CCm1ΨCCAGACAm1ΨACGm1ΨGACCCAGCAGC | | |
| m1ΨGAm1ΨCAGAGCCGCCGAGAm1Ψm1ΨAGA | | |
| GCCm1ΨCm1ΨGCCAAm1ΨCm1ΨGGCCGCCAC | | |
| CAAGAm1ΨGm1ΨCm1ΨGAGm1ΨGm1ΨGm1ΨG | | |
| Cm1ΨGGGCCAGAGCAAGAGAGm1ΨGGACm1Ψ | | |
| m1Ψm1Ψm1ΨGCGGCAAGGGCm1ΨACCACCm1 | | |
| ΨGAm1ΨGAGCm1Ψm1ΨCCCm1ΨCAGm1ΨCm1 | | |
| ΨGCCCCm1ΨCACGGCGm1ΨGGm1ΨGm1Ψm1Ψ | | |
| m1ΨCm1ΨGCACGm1ΨGACAm1ΨAm1ΨGm1ΨG | | |
| CCCGCm1ΨCAAGAGAAGAAm1Ψm1Ψm1ΨCACC | | |
| ACCGCm1ΨCCAGCCAm1ΨCm1ΨGCCACGACG | | |
| GCAAAGCCCACm1Ψm1Ψm1ΨCCm1ΨAGAGAAG | | |
| GCGm1ΨGm1Ψm1ΨCGm1ΨGm1ΨCCAACGGCA | | |
| CCCAm1Ψm1ΨGGm1Ψm1ΨCGm1ΨGACACAGC | | |
| GGAACm1Ψm1ΨCm1ΨACGAGCCCCAGAm1ΨCA | | |
| m1ΨCACCACCGACAACACCm1Ψm1ΨCGm1ΨG | | |
| m1ΨCm1ΨGGCAACm1ΨGCGACGm1ΨCGm1ΨG | | |
| Am1ΨCGGCAm1Ψm1ΨGm1ΨGAACAAm1ΨACC | | |
| Gm1ΨGm1ΨACGACCCm1ΨCm1ΨGCAGCCCGA | | |
| GCm1ΨGGACAGCm1Ψm1ΨCAAAGAGGAACm1 | | |
| ΨGGACAAGm1ΨACm1Ψm1Ψm1ΨAAGAACCACA | | |
| CAAGCCCCGACGm1ΨGGACCm1ΨGGGCGAm1 | | |
| ΨAm1ΨCAGCGGAAm1ΨCAAm1ΨGCCAGCGm1 | | |
| ΨCGm1ΨGAACAm1ΨCCAGAAAGAGAm1ΨCGAC | | |
| CGGCm1ΨGAACGAGGm1ΨGGCCAAGAAm1ΨC | | |
| m1ΨGAACGAGAGCCm1ΨGAm1ΨCGACCm1ΨG | | |
| CAAGAACm1ΨGGGGAAGm1ΨACGAGCAGm1ΨA | | |
| CAm1ΨCAAGm1ΨGGCCCm1ΨGGm1ΨACAm1Ψ | | |
| Cm1ΨGGCm1ΨGGGCm1Ψm1Ψm1ΨAm1ΨCGCC | | |
| GGACm1ΨGAm1Ψm1ΨGCCAm1ΨCGm1ΨGAm1 | | |
| ΨGGm1ΨCACAAm1ΨCAm1ΨGCm1ΨGm1ΨGm1 | | |
| Ψm1ΨGCAm1ΨGACCAGCm1ΨGCm1ΨGm1ΨAG | | |
| Cm1ΨGCCm1ΨGAAGGGCm1ΨGm1Ψm1ΨGm1Ψ | | |
| AGCm1ΨGm1ΨGGCAGCm1ΨGCm1ΨGCAAGm1 | | |
| Ψm1ΨCGACGAGGACGAm1Ψm1ΨCm1ΨGAGCC | | |
| CGm1ΨGCm1ΨGAAGGGCGm1ΨGAAACm1ΨGC | | |
| ACm1ΨACACAm1ΨGAm1ΨGACm1ΨCGAGCm1Ψ | | |
| GGm1ΨACm1ΨGCAm1ΨGCACGCAAm1ΨGCm1 | | |
| ΨAGCm1ΨGCCCCm1Ψm1Ψm1ΨCCCGm1ΨCCm | | |
| 1ΨGGGm1ΨACCCCGAGm1ΨCm1ΨCCCCCGAC | | |
| Cm1ΨCGGGm1ΨCCCAGGm1ΨAm1ΨGCm1ΨCC | | |
| CACCm1ΨCCACCm1ΨGCCCCACm1ΨCACCACC | | |
| m1ΨCm1ΨGCm1ΨAGm1Ψm1ΨCCAGACACCm1 | | |
| ΨCCCAAGCACGCAGCAAm1ΨGCAGCm1ΨCAAA | | |
| ACGCm1Ψm1ΨAGCCm1ΨAGCCACACCCCCACG | | |
| GGAAACAGCAGm1ΨGAm1Ψm1ΨAACCm1Ψm1Ψ | | |
| m1ΨAGCAAm1ΨAAACGAAAGm1Ψm1Ψm1ΨAAC | | |
| m1ΨGGm1ΨCAAm1Ψm1Ψm1ΨCGm1ΨGCCAGC | | |
| m1ΨAAGCm1ΨAm1ΨACm1ΨAACCCCAGGGm1Ψ | | |
| m1ΨGGm1ΨCAAm1Ψm1Ψm1ΨCGm1ΨGCCAGC | | |
| CACACCCm1ΨGGAGCm1ΨAGCAAAAAAAAAAA | | |
| AAAAAAAAAAAAAAAAAAA | | |
| GAGAAm1ΨAAACm1ΨAGm1ΨAm1Ψm1ΨCm1Ψ | Modified BNT162b2 | 7 |
| m1ΨCm1ΨGGm1ΨCCCCACAGACm1ΨCAGAGA | | |
| GAACCCGCCACCAm1ΨGm1Ψm1ΨCGm1ΨGm1 | | |
| Ψm1ΨCCm1ΨGGm1ΨGCm1ΨGCm1ΨGCCm1ΨC | | |
| m1ΨGGm1ΨGm1ΨCCAGCCAGm1ΨGm1ΨGm1Ψ | | |
| GAACCm1ΨGACCACCAGAACACAGCm1ΨGCCm | | |
| 1ΨCCAGCCm1ΨACACCAACAGCm1Ψm1Ψm1ΨA | | |
| CCAGAGGCGm1ΨGm1ΨACm1ΨACCCCGACAA | | |
| GGm1ΨGm1Ψm1ΨCAGAm1ΨCCAGCGm1ΨGCm | | |
| 1ΨGCACm1ΨCm1ΨACCCAGGACCm1ΨGm1Ψm1 | | |
| ΨCCm1ΨGCCm1Ψm1Ψm1ΨCm1Ψm1ΨCAGCAA | | |
| CGm1ΨGACCm1ΨGGm1Ψm1ΨCCACGCCAm1Ψ | | |
| CCACGm1ΨGm1ΨCCGGCACCAAm1ΨGGCACC | | |
| AAGAGAm1Ψm1ΨCGACAACCCCGm1ΨGCm1Ψ | | |
| GCCCm1Ψm1ΨCAACGACGGGGm1ΨGm1ΨACm | | |
| 1Ψm1Ψm1ΨGCCAGCACCGAGAAGm1ΨCCAACA | | |
| m1ΨCAm1ΨCAGAGGCm1ΨGGAm1ΨCm1Ψm1Ψ | | |
| CGGCACCACACm1ΨGGACAGCAAGACCCAGAG | | |
| CCm1ΨGCm1ΨGAm1ΨCGm1ΨGAACAACGCCA | | |
| CCAACGm1ΨGGm1ΨCAm1ΨCAAAGm1ΨGm1Ψ | | |
| GCGAGm1Ψm1ΨCCAGm1Ψm1ΨCm1ΨGCAACG | | |
| ACCCCm1Ψm1ΨCCm1ΨGGGCGm1ΨCm1ΨACm | | |
| 1ΨACCACAAGAACAACAAGAGCm1ΨGGAm1ΨG | | |
| GAAAGCGAGm1Ψm1ΨCCGGGm1ΨGm1ΨACAG | | |
| CAGCGCCAACAACm1ΨGCACCm1Ψm1ΨCGAG | | |
| m1ΨACGm1ΨGm1ΨCCCAGCCm1Ψm1Ψm1ΨCC | | |
| m1ΨGAm1ΨGGACCm1ΨGGAAGGCAAGCAGGG | | |
| CAACm1Ψm1ΨCAAGAACCm1ΨGCGCGAGm1Ψ | | |
| m1ΨCGm1ΨGm1Ψm1Ψm1ΨAAGAACAm1ΨCGA | | |
| CGGCm1ΨACm1Ψm1ΨCAAGAm1ΨCm1ΨACAG | | |
| CAAGCACACCCCm1ΨAm1ΨCAACCm1ΨCGm1Ψ | | |
| GCGGGAm1ΨCm1ΨGCCm1ΨCAGGGCm1Ψm1Ψ | | |
| Cm1ΨCm1ΨGCm1ΨCm1ΨGGAACCCCm1ΨGGm | | |
| 1ΨGGAm1ΨCm1ΨGCCCAm1ΨCGGCAm1ΨCAA | | |
| CAm1ΨCACCCGGm1Ψm1ΨcCAGACACm1ΨGCm | | |
| 1ΨGGCCCm1ΨGCACAGAAGCm1ΨACCm1ΨGAC | | |
| ACCm1ΨGGCGAm1ΨAGCAGCAGCGGAm1ΨGG | | |
| ACAGCm1ΨGGm1ΨGCCGCCGCm1Ψm1ΨACm1 | | |
| ΨAm1ΨGm1ΨGGGCm1ΨACCm1ΨGCAGCCm1Ψ | | |
| AGAACCm1Ψm1ΨCCm1ΨGCm1ΨGAAGm1ΨACA | | |
| ACGAGAACGGCACCAm1ΨCACCGACGCCGm1Ψ | | |
| GGAm1Ψm1ΨGm1ΨGCm1ΨCm1ΨGGAm1ΨCCm | | |
| 1ΨCm1ΨGAGCGAGACAAAGm1ΨGCACCCm1ΨG | | |
| AAGm1ΨCCm1Ψm1ΨCACCGm1ΨGGAAAAGGG | | |
| CAm1ΨCm1ΨACCAGACCAGCAACm1Ψm1ΨCCG | | |
| GGm1ΨGCAGCCCACCGAAm1ΨCCAm1ΨCGm1 | | |
| ΨGCGGm1Ψm1ΨCCCCAAm1ΨAm1ΨCACCAAm1 | | |
| ΨCm1ΨGm1ΨGCCCCm1Ψm1ΨCGGCGAGGm1Ψ | | |
| Gm1Ψm1ΨCAAm1ΨGCCACCAGAm1Ψm1ΨCGC | | |
| Cm1ΨCm1ΨGm1ΨGm1ΨACGCCm1ΨGGAACCG | | |
| GAAGCGGAm1ΨCAGCAAm1Ψm1ΨGCGm1ΨGG | | |
| CCGACm1ΨACm1ΨCCGm1ΨGCm1ΨGm1ΨACA | | |
| ACm1ΨCCGCCAGCm1Ψm1ΨCAGCACCm1Ψm1 | | |
| ΨCAAGm1ΨGCm1ΨACGGCGm1ΨGm1ΨCCCCm | | |
| 1ΨACCAAGCm1ΨGAACGACCm1ΨGm1ΨGCm1Ψ | | |
| m1ΨCACAAACGm1ΨGm1ΨACGCCGACAGCm1Ψ | | |
| m1ΨCGm1ΨGAm1ΨCCGGGGAGAm1ΨGAAGm1 | | |
| ΨGCGGCAGAm1Ψm1ΨGCCCCm1ΨGGACAGAC | | |
| AGGCAAGAm1ΨCGCCGACm1ΨACAACm1ΨACA | | |
| AGCm1ΨGCCCGACGACm1Ψm1ΨCACCGGCm1 | | |
| ΨGm1ΨGm1ΨGAm1Ψm1ΨGCCm1ΨGGAACAGC | | |
| AACAACCm1ΨGGACm1ΨCCAAAGm1ΨCGGCGG | | |
| CAACm1ΨACAAm1Ψm1ΨACCm1ΨGm1ΨACCGG | | |
| Cm1ΨGm1Ψm1ΨCCGGAAGm1ΨCCAAm1ΨCm1 | | |
| ΨGAAGCCCm1Ψm1ΨCGAGCGGGACAm1ΨCm1 | | |
| ΨCCACCGAGAm1ΨCm1ΨAm1ΨCAGGCCGGCA | | |
| GCACCCCm1Ψm1ΨGm1ΨAACGGCGm1ΨGGAA | | |
| GGCm1Ψm1ΨCAACm1ΨGCm1ΨACm1Ψm1ΨCC | | |
| CACm1ΨGCAGm1ΨCCm1ΨACGGCm1Ψm1ΨcCA | | |
| GCCCACAAAm1ΨGGCGm1ΨGGGCm1ΨAm1ΨC | | |
| AGCCCm1ΨACAGAGm1ΨGGm1ΨGGm1ΨGCm1 | | |
| ΨGAGCm1Ψm1ΨCGAACm1ΨGCm1ΨGCAm1ΨG | | |
| CCCCm1ΨGCCACAGm1ΨGm1ΨGCGGCCCm1Ψ | | |
| AAGAAAAGCACCAAm1ΨCm1ΨCGm1ΨGAAGAA | | |
| CAAAm1ΨGCGm1ΨGAACm1Ψm1ΨCAACm1Ψm1 | | |
| ΨCAACGGCCm1ΨGACCGGCACCGGCGm1ΨGC | | |
| m1ΨGACAGAGAGCAACAAGAAGm1Ψm1ΨCCm1 | | |
| ΨGCCAm1Ψm1ΨCCAGCAGm1Ψm1Ψm1ΨGGCC | | |
| GGGAm1ΨAm1ΨCGCCGAm1ΨACCACAGACGC | | |
| CGm1Ψm1ΨAGAGAm1ΨCCCCAGACACm1ΨGG | | |
| AAAm1ΨCCm1ΨGGACAm1ΨCACCCCm1Ψm1ΨG | | |
| CAGCm1Ψm1ΨCGGCGGAGm1ΨGm1ΨCm1ΨGm | | |
| 1ΨGAm1ΨCACCCCm1ΨGGCACCAACACCAGCA | | |
| Am1ΨCAGGm1ΨGGCAGm1ΨGCm1ΨGm1ΨACC | | |
| AGGACGm1ΨGAACm1ΨGm1ΨACCGAAGm1ΨG | | |
| CCCGm1ΨGGCCAm1Ψm1ΨCACGCCGAm1ΨCA | | |
| GCm1ΨGACACCm1ΨACAm1ΨGGCGGGm1ΨGm | | |
| 1ΨACm1ΨCCACCGGCAGCAAm1ΨGm1ΨGm1Ψ | | |
| m1ΨcCAGACCAGAGCCGGCm1ΨGm1ΨCm1ΨGA | | |
| m1ΨCGGAGCCGAGCACGm1ΨGAACAAm1ΨAG | | |
| Cm1ΨACGAGm1ΨGCGACAm1ΨCCCCAm1ΨCG | | |
| GCGCm1ΨGGAAm1ΨCm1ΨGCGCCAGCm1ΨAC | | |
| CAGACACAGACAAACAGCCCm1ΨCGGAGAGCC | | |
| AGAAGCGm1ΨGGCCAGCCAGAGCAm1ΨCAm1 | | |
| Ψm1ΨGCCm1ΨACACAAm1ΨGm1ΨCm1ΨCm1Ψ | | |
| GGGCGCCGAGAACAGCGm1ΨGGCCm1ΨACm1 | | |
| ΨCCAACAACm1ΨCm1ΨAm1ΨCGCm1ΨAm1ΨCC | | |
| CCACCAACm1Ψm1ΨCACCAm1ΨCAGCGm1ΨGA | | |
| CCACAGAGAm1ΨCCm1ΨGCCm1ΨGm1ΨGm1Ψ | | |
| CCAm1ΨGACCAAGACCAGCGm1ΨGGACm1ΨG | | |
| CACCAm1ΨGm1ΨACAm1ΨCm1ΨGCGGCGAm1 | | |
| Ψm1ΨCCACCGAGm1ΨGCm1ΨCCAACCm1ΨGC | | |
| m1ΨGCm1ΨGCAGm1ΨACGGCAGCm1Ψm1ΨCm | | |
| 1ΨGCACCCAGCm1ΨGAAm1ΨAGAGCCCm1ΨGA | | |
| CAGGGAm1ΨCGCCGm1ΨGGAACAGGACAAGAA | | |
| CACCCAAGAGGm1ΨGm1Ψm1ΨCGCCCAAGm1 | | |
| ΨGAAGCAGAm1ΨCm1ΨACAAGACCCCm1ΨCC | | |
| m1ΨAm1ΨCAAGGACm1Ψm1ΨCGGCGGCm1Ψm | | |
| 1ΨCAAm1Ψm1Ψm1ΨCAGCCAGAm1Ψm1ΨCm1Ψ | | |
| GCCCGAm1ΨCCm1ΨAGCAAGCCCAGCAAGCG | | |
| GAGCm1Ψm1ΨCAm1ΨCGAGGACCm1ΨGCm1Ψ | | |
| Gm1Ψm1ΨCAACAAAGm1ΨGACACm1ΨGGCCGA | | |
| CGCCGGCm1Ψm1ΨCAm1ΨCAAGCAGm1ΨAm1 | | |
| ΨGGCGAm1Ψm1ΨGm1ΨCm1ΨGGGCGACAm1Ψ | | |
| m1ΨGCCGCCAGGGAm1ΨCm1ΨGAm1Ψm1Ψm1 | | |
| ΨGCGCCCAGAAGm1Ψm1Ψm1ΨAACGGACm1Ψ | | |
| GACAGm1ΨGCm1ΨGCCm1ΨCCm1ΨCm1ΨGCm | | |
| 1ΨGACCGAm1ΨGAGAm1ΨGAm1ΨCGCCCAGm1 | | |
| ΨACACAm1ΨCm1ΨGCCCm1ΨGCm1ΨGGCCGG | | |
| CACAAm1ΨCACAAGCGGCm1ΨGGACAm1Ψm1Ψ | | |
| m1ΨGGAGCAGGCGCCGCm1ΨCm1ΨGCAGAm1 | | |
| ΨCCCCm1Ψm1Ψm1ΨGCm1ΨAm1ΨGCAGAm1Ψ | | |
| GGCCm1ΨACCGGm1Ψm1ΨCAACGGCAm1ΨCG | | |
| GAGm1ΨGACCCAGAAm1ΨGm1ΨGCm1ΨGm1Ψ | | |
| ACGAGAACCAGAAGCm1ΨGAm1ΨCGCCAACCA | | |
| Gm1Ψm1ΨCAACAGCGCCAm1ΨCGGCAAGAm1 | | |
| ΨCCAGGACAGCCm1ΨGAGCAGCACAGCAAGC | | |
| GCCCm1ΨGGGAAAGCm1ΨGCAGGACGm1ΨGG | | |
| m1ΨCAACCAGAAm1ΨGCCCAGGCACm1ΨGAAC | | |
| ACCCm1ΨGGm1ΨCAAGCAGCm1ΨGm1ΨCCm1 | | |
| ΨCCAACm1Ψm1ΨCGGCGCCAm1ΨCAGCm1ΨC | | |
| m1ΨGm1ΨGCm1ΨGAACGAm1ΨAm1ΨCCm1ΨG | | |
| AGCAGACm1ΨGGACCCm1ΨCCm1ΨGAGGCCG | | |
| AGGm1ΨGCAGAm1ΨCGACAGACm1ΨGAm1ΨC | | |
| ACAGGCAGACm1ΨGCAGAGCCm1ΨCCAGACAm | | |
| 1ΨACGm1ΨGACCCAGCAGCm1ΨGAm1ΨCAGAG | | |
| CCGCCGAGAm1Ψm1ΨAGAGCCm1ΨCm1ΨGCC | | |
| AAm1ΨCm1ΨGGCCGCCACCAAGAm1ΨGm1ΨC | | |
| m1ΨGAGm1ΨGm1ΨGm1ΨGCm1ΨGGGCCAGAG | | |
| CAAGAGAGm1ΨGGACm1Ψm1Ψcm1ΨGCGGCAA | | |
| GGGCm1ΨACCACCm1ΨGAm1ΨGAGCm1Ψm1Ψ | | |
| CCCm1ΨCAGm1ΨCm1ΨGCCCCm1ΨCACGGCG | | |
| m1ΨGGm1ΨGm1Ψm1ΨcCm1ΨGCACGm1ΨGAC | | |
| Am1Ψ'Am1ΨGm1ΨGCCCGCm1ΨCAAGAGAAGAA | | |
| m1Ψm1Ψm1ΨCACCACCGCm1ΨCCAGCCAm1Ψ | | |
| Cm1ΨGCCACGACGGCAAAGCCCACm1Ψm1ΨcC | | |
| Cm1ΨAGAGAAGGCGm1ΨGm1Ψm1ΨCGm1ΨGm | | |
| 1ΨCCAACGGCACCCAm1Ψm1ΨGGm1Ψm1ΨCG | | |
| m1ΨGACACAGCGGAACm1Ψm1ΨCm1ΨACGAG | | |
| CCCCAGAm1ΨCAm1ΨCACCACCGACAACACCm | | |
| 1Ψm1ΨCGm1ΨGm1ΨCm1ΨGGCAACm1ΨGCGA | | |
| CGm1ΨCGm1ΨGAm1ΨCGGCAm1Ψm1ΨGm1ΨG | | |
| AACAAm1ΨACCGm1ΨGm1ΨACGACCCm1ΨCm1 | | |
| ΨGCAGCCCGAGCm1ΨGGACAGCm1Ψm1ΨCAA | | |
| AGAGGAACm1ΨGGACAAGm1ΨACm1Ψm1Ψm1 | | |
| ΨAAGAACCACACAAGCCCCGACGm1ΨGGACCm | | |
| 1ΨGGGCGAm1ΨAm1ΨCAGCGGAAm1ΨCAAm1 | | |
| ΨGCCAGCGm1ΨCGm1ΨGAACAm1ΨCCAGAAA | | |
| GAGAm1ΨCGACCGGCm1ΨGAACGAGGm1ΨGG | | |
| CCAAGAAm1ΨCm1ΨGAACGAGAGCCm1ΨGAm1 | | |
| ΨCGACCm1ΨGCAAGAACm1ΨGGGGAAGm1ΨA | | |
| CGAGCAGm1ΨACAm1ΨCAAGm1ΨGGCCCm1Ψ | | |
| GGm1ΨACAm1ΨCm1ΨGGCm1ΨGGGCm1Ψm1Ψ | | |
| m1ΨAm1ΨCGCCGGACm1ΨGAm1Ψm1ΨGCCAm | | |
| 1ΨCGm1ΨGAm1ΨGGm1ΨCACAAm1ΨCAm1ΨG | | |
| Cm1ΨGm1ΨGm1Ψm1ΨGCAm1ΨGACCAGCm1Ψ | | |
| GCm1ΨGm1ΨAGCm1ΨGCCm1ΨGAAGGGCm1Ψ | | |
| Gm1Ψm1ΨGm1ΨAGCm1ΨGm1ΨGGCAGCm1ΨG | | |
| Cm1ΨGCAAGm1Ψm1ΨCGACGAGGACGAm1Ψm | | |
| 1ΨCm1ΨGAGCCCGm1ΨGCm1ΨGAAGGGCGm1 | | |
| ΨGAAACm1ΨGCACm1ΨACACAm1ΨGAm1ΨGAC | | |
| m1ΨCGAGCm1ΨGGm1ΨACm1ΨGCAm1ΨGCAC | | |
| GCAAm1ΨGCm1ΨAGCm1ΨGCCCCm1Ψm1ΨcCC | | |
| CGm1ΨCCm1ΨGGGm1ΨACCCCGAGm1ΨCm1Ψ | | |
| CCCCCGACCm1ΨCGGGm1ΨCCCAGGm1ΨAm1 | | |
| ΨGCm1ΨCCCACCm1ΨCCACCm1ΨGCCCCACm | | |
| 1ΨCACCACCm1ΨCm1ΨGCm1ΨAGm1Ψm1ΨCCA | | |
| GACACCm1ΨCCCAAGCACGCAGCAAm1ΨGCAG | | |
| Cm1ΨCAAAACGCm1Ψm1ΨAGCCm1ΨAGCCACA | | |
| CCCCCACGGGAAACAGCAGm1ΨGAm1Ψm1ΨAA | | |
| CCm1Ψm1Ψm1ΨAGCAAm1ΨAAACGAAAGm1Ψm | | |
| 1Ψm1ΨAACm1ΨAAGCm1ΨAm1ΨACm1ΨAACCC | | |
| CAGGGm1Ψm1ΨGGm1ΨCAAm1Ψm1Ψm1ΨCGm | | |
| 1ΨGCCAGCCACACCCm1ΨGGAGCm1ΨAGCAAA | | |
| AAAAAAAAAAAAAAAAAAAAAAAAAAA | | |
| ATGGGTGACTACAAAGACCATGACGGTGATTATA | T208C | 8 |
| AAGATCATGACATCGATTACAAGGATGACGATGA | | |
| CAAGCTCGAAGACGCCAAAAACATAAAGAAAGG | | |
| CCCGGCGCCATTCTATCCGCTGGAAGATGGAAC | | |
| CGCTGGAGAGCAACTGCATAAGGCTATGAAGAG | | |
| ATACGCCCTGGTTCCTGGAACAATTGCTTTTACA | | |
| GATGCACATATCGAGGTGGACATCACTTACGCTG | | |
| AGTACTTCGAAATGTCCGTTCGGTTGGCAGAAG | | |
| CTATGAAACGATATGGGCTGAATACAAATCACAG | | |
| AATCGTCGTATGCAGTGAAAACTCTCTTCAATTC | | |
| TTTATGCCGGTGTTGGGCGCGTTATTTATCGGAG | | |

ACGTGAATTGCTCAACAGTATGGGCATTTCGCA

| | | |
|---|---|---|
| TTGCAGTTGCGCCCGCGAACGACATTTATAATGA | | |
| ACGTGAATTGCTCAACAGTATGGGCATTTCGCA | | |
| GCCTACCGTGGTGTTCGTTTCCAAAAAGGGGTT | | |
| GCAAAAAATTTTGAACGTGCAAAAAAAGCTCCCA | | |
| ATCATCCAAAAAATTATTATCATGGATTCTAAAAC | | |
| GGATTACCAGGGATTTCAGTCGATGTACACGTTC | | |
| GTCACATCTCATCTACCTCCCGGTTTTAATGAATA | | |
| CGATTTTGTGCCAGAGTCCTTCGATAGGGACAA | | |
| GACAATTGCACTGATCATGAACTCCTCTGGATCT | | |
| ACTGGTCTGCCTAAAGGTGTCGCTCTGCCTCAT | | |
| AGAACTGCCTGCGTGAGATTCTCGCATGCCAGA | | |
| GATCCTATTTTTCGGCAATCAAATCATTCCGGATA | | |
| CTGCGATTTTAAGTGTTGTTCCATTCCATCACGG | | |
| TTCTGGAATGTTTACTACACTOGGATATTTGATAT | | |
| GTGGATTTCGAGTCGTCTTAATGTATAGATTTGAA | | |
| GAAGAGCTGTTTCTGAGGAGCCTTCAGGATTAC | | |
| AAGATTCAAAGTGCGCTGCTGGTGCCAACCCTA | | |
| TTCTCCTTCTTCGCCAAAAGCACTCTGATTGACA | | |
| AATACGATTTATCTAATTTACACGAAATTGCTTCT | | |
| GGTGGCGCTCCCCTCTCTAAGGAAGTCGGGGA | | |
| AGCGGTTGCCAAGAGGTTCCATCTGCCAGGTAT | | |
| CAGGCAAGGATATGGGCTCACTGAGACTACATC | | |
| AGCTATTCTGATTACACCCGAGGGGGATGATAAA | | |
| CCGGGCGCGGTCGGTAAAGTTGTTCCATTTTTT | | |
| GAAGCGAAGGTTGTGGATCTGGATACCGGGAAA | | |
| ACGCTGGGCGTTAATCAAAGAGGCGAACTGTGT | | |
| GTGAGAGGTCCTATGATTATGTCCGGTTATGTAA | | |
| ACAATCCGGAAGCGACCAACGCCTTGATTGACA | | |
| AGGATGGATGGCTACATTCTGGAGACATAGCTTA | | |
| CTGGGACGAAGACGAACACTTCTTCATCGTTGA | | |
| CCGCCTGAAGTCTCTGATTAAGTACAAAGGCTAT | | |
| CAGGTGGCTCCCGCTGAATTGGAATCCATCTTG | | |
| CTCCAACACCCCAACATCTTCGACGCAGGTGTC | | |
| GCAGGTCTTCCCGACGATGACGCCGGTGAACTT | | |
| CCCGCCGCCGTTGTTGTTTTGGAGCACGGAAA | | |
| GACGATGACGGAAAAAGAGATCGTGGATTACGT | | |
| CGCCAGTCAAGTAACAACCGCGAAAAAGTTGCG | | |
| CGGAGGAGTTGTGTTTGTGGACGAAGTACCGAA | | |
| AGGTCTTACCGGAAAACTCGACGCAAGAAAAAT | | |
| CAGAGAGATCCTCATAAAGGCCAAGAAGGGCGG | | |
| AAAGATCGCCGTG | | |
| ATGGGTGACTACAAAGACCATGACGGTGATTATA | AntiFS | 9 |
| AAGATCATGACATAGATTACAAGGATGACGATGA | | |
| CAAGCTCGAAGACGCCAAAAACATAAAGAAAGG | | |
| CCCGGCGCCATTCTATCCGCTAGAAGATGGAAC | | |
| CGCTGGAGAGCAACTGCATAAGGCTATGAAGAG | | |
| ATACGCCCTAGTTCCTGGAACAATAGCTTTTACA | | |
| GATGCACATATAGAGGTAGACATCACTTACGCTG | | |
| AGTACTTCGAAATGAGCGTAAGGTTGGCAGAAG | | |
| CTATGAAACGATATGGGCTGAATACAAATCACAG | | |
| AATAGTCGTAAGCAGTGAAAACTCTCTTCAATTC | | |
| TTTATGCCGGTGTTAGGCGCGTTATTTATCGGAG | | |
| TAGCAGTAGCGCCCGCGAACGACATTTATAATGA | | |
| ACGTGAATTGCTAAACAGTATGGGCATTTCGCAG | | |
| CCTACCGTAGTGTTCGTAGCCAAAAAGGGGTTG | | |
| CAAAAAATTTTGAACGTGCAAAAAAAGCTCCCAA | | |
| TCATCCAAAAAATTATTATAATGGATTCTAAAATTT | | |
| TGAACGTGCAAAAAAAGCTCCCAATCATCCAAAA | | |
| AATTATTATAATGGATTCTAAAATTAATGAATACGA | | |
| TTTTGTGCCAGAGTCCTTCGATAGGGACAAGAC | | |
| AATAGCACTGATAATGAACTCCTCTGGATCTACT | | |
| GGTCTGCCTAAAGGTGTAGCTCTGCCTCATAGA | | |
| ACTGCCTGCGTGAGATTCTCGCATGCCAGAGAT | | |
| CCTATTTTTCGGCAATCAAATCATTCCGGATACTG | | |
| CGATTTTAAGTGTTGTTCCATTCCATCACGGTTTT | | |
| GGAATGTTTACTACACTCGGATATTTGATATGTGG | | |
| ATAACGAGTCGTCTAAATGTATAGATTTGAAGAA | | |
| GAGCTGTTTCTGAGGAGCCTTCAGGATTACAAG | | |
| ATTCAAAGTGCGCTGCTGGTGCCAACCCTATTCT | | |
| CCTTCTTCGCCAAAAGCACTCTGATTGACAAATA | | |
| CGATTTATCTAATTTACACGAAATTGCTTCTGGTG | | |
| GCGCTCCCCTCTCTAAGGAAGTCGGGGAAGCG | | |
| GTTGCCAAGAGGTTCCATCTGCCAGGTATCAGG | | |
| CAAGGATATGGGCTCACTGAGACTACATCAGCTA | | |
| TTCTGATTACACCCGAGGGGGATGATAAACCGG | | |
| GCGCGGTCGGTAAAGTTGTTCCATTTTTTGAAG | | |
| CGAAGGTTGTGGATCTGGATACCGGGAAAACGC | | |
| TGGGCGTTAATCAAAGAGGCGAACTGTGTGTGA | | |
| GAGGTCCTATGATTATGTCCGGTTATGTAAACAAT | | |
| CCGGAAGCGACCAACGCCTTGATTGACAAGGAT | | |
| GGATGGCTACATTCTGGAGACATAGCTTACTGG | | |
| GACGAAGACGAACACTTCTTCATCGTTGACCGC | | |
| CTGAAGTCTCTGATTAAGTACAAAGGCTATCAGG | | |
| TGGCTCCCGCTGAATTGGAATCCATCTTGCTCC | | |
| AACACCCCAACATCTTCGACGCAGGTGTCGCAG | | |
| GTCTTCCCGACGATGACGCCGGTGAACTTCCCG | | |
| CCGCCGTTGTTGTTTTGGAGCACGGAAAGACGA | | |
| TGACGGAAAAAGAGATCGTGGATTACGTCGCCA | | |
| GTCAAGTAACAACCGCGAAAAAGTTGCGCGGAG | | |
| GAGTTGTGTTTGTGGACGAAGTACCGAAAGGTC | | |
| TTACCGGAAAACTCGACGCAAGAAAAATCAGAG | | |
| AGATCCTCATAAAGGCCAAGAAGGGCGGAAAGA | | |
| TCGCCGTG | | |
| MFVFLVLLPLVSSQCVNL | Spike | 10 |
| PLVSSQCVNLTTRTQL | Spike | 11 |
| NLTTRTQLPPAYTNSFTR | Spike | 12 |
| PPAYTNSFTRGVYYPDKV | Spike | 13 |
| TRGVYYPDKVFRSSVL | Spike | 14 |
| KVFRSSVLHSTQDLFLPF | Spike | 15 |
| HSTQDLFLPFFSNVTWF | Spike | 16 |
| LPFFSNVTWFHAIHV | Spike | 17 |
| FHAIHVSGTNGTKRF | Spike | 18 |
| TNGTKRFDNPVLPFNDGV | Spike | 19 |
| NPVLPFNDGVYFASTEK | Spike | 20 |
| GVYFASTEKSNIIRGWIF | Spike | 21 |
| KSNIIRGWIFGTTLDSK | Spike | 22 |
| IFGTTLDSKTQSLLIV | Spike | 23 |
| KTQSLLIVNNATNVVIKV | Spike | 24 |
| NNATNVVIKVCEFQF | Spike | 25 |
| KVCEFQFCNDPFLGVYY | Spike | 26 |
| NDPFLGVYYHKNNKSWM | Spike | 27 |
| YHKNNKSWMESEFRVY | Spike | 28 |
| MESEFRVYSSANNCTFEY | Spike | 29 |
| SSANNCTFEYVSQPFLM | Spike | 30 |
| EYVSQPFLMDLEGKQGNF | Spike | 31 |
| MDLEGKQGNFKNLREFVF | Spike | 32 |
| NFKNLREFVFKNIDGYFK | Spike | 33 |
| VFKNIDGYFKIYSKHTPI | Spike | 34 |
| FKIYSKHTPINLVRDL | Spike | 35 |
| PINLVRDLPQGFSALEPL | Spike | 36 |
| PQGFSALEPLVDLPIGI | Spike | 37 |
| PLVDLPIGINITRFQTLL | Spike | 38 |
| INITRFQTLLALHRSYL | Spike | 39 |
| LLALHRSYLTPGDSSSGW | Spike | 40 |
| LTPGDSSSGWTAGAAAYY | Spike | 41 |
| GWTAGAAAYYVGYLQPR | Spike | 42 |
| YYVGYLQPRTFLLKY | Spike | 43 |
| RTFLLKYNENGTITDAV | Spike | 44 |
| ENGTITDAVDCALDPL | Spike | 45 |
| VDCALDPLSETKCTLKSF | Spike | 46 |
| SETKCTLKSFTVEKGIY | Spike | 47 |
| SFTVEKGIYQTSNFRV | Spike | 48 |
| YQTSNFRVQPTESIVTF | Spike | 49 |
| QPTESIVTFPNITNLCPF | Spike | 50 |
| FPNITNLCPFGEVFNATR | Spike | 51 |
| PFGEVFNATRFASVYAW | Spike | 52 |
| TRFASVYAWNRKRISNCV | Spike | 53 |
| WNRKRISNCVADYSVLY | Spike | 54 |
| CVADYSVLYNSASFSTFK | Spike | 55 |
| YNSASFSTFKCYGVSPTK | Spike | 56 |
| FKCYGVSPTKLNDLCF | Spike | 57 |
| TKLNDLCFTNVYADSFVI | Spike | 58 |
| TNVYADSFVIRGDEVRQI | Spike | 59 |
| VIRGDEVRQIAPGQTGKI | Spike | 60 |
| QIAPGQTGKIADYNYKL | Spike | 61 |
| KIADYNYKLPDDFTGCVI | Spike | 62 |
| LPDDFTGCVIAWNSNNL | Spike | 63 |
| VIAWNSNNLDSKVGGNY | Spike | 64 |
| LDSKVGGNYNYLYRLFRK | Spike | 65 |
| YNYLYRLFRKSNLKPFER | Spike | 66 |
| RKSNLKPFERDISTEIY | Spike | 67 |
| ERDISTEIYQAGSTPC | Spike | 68 |
| YQAGSTPCNGVEGENCYF | Spike | 69 |
| NGVEGFNCYFPLQSYGF | Spike | 70 |
| YFPLQSYGFQPTNGVGY | Spike | 71 |
| FQPTNGVGYQPYRVVVL | Spike | 72 |
| GYQPYRVVVLSFELL | Spike | 73 |
| LSFELLHAPATVCGPKK | Spike | 74 |
| PATVCGPKKSTNLVKNK | Spike | 75 |
| KSTNLVKNKCVNFNFNGL | Spike | 76 |
| KCVNFNFNGLTGTGVL | Spike | 77 |
| GLTGTGVLTESNKKFLPF | Spike | 78 |
| TESNKKFLPFQQFGRDI | Spike | 79 |
| PFQQFGRDIADTTDAVR | Spike | 80 |
| IADTTDAVRDPQTLEIL | Spike | 81 |
| RDPQTLEILDITPCSF | Spike | 82 |
| ILDITPCSFGGVSVI | Spike | 83 |
| GGVSVITPGTNTSNQVAV | Spike | 84 |
| GTNTSNQVAVLYQDV | Spike | 85 |
| AVLYQDVNCTEVPVAI | Spike | 86 |
| CTEVPVAIHADQLTPTWR | Spike | 87 |
| HADQLTPTWRVYSTGSNV | Spike | 88 |
| WRVYSTGSNVFQTRAGCL | Spike | 89 |
| NVFQTRAGCLIGAEHV | Spike | 90 |
| CLIGAEHVNNSYECDIPI | Spike | 91 |
| NNSYECDIPIGAGICASY | Spike | 92 |
| PIGAGICASYQTQTNSPR | Spike | 93 |
| SYQTQTNSPRRARSV | Spike | 94 |
| PRRARSVASQSIIAYTM | Spike | 95 |
| SQSIIAYTMSLGAENSV | Spike | 96 |
| MSLGAENSVAYSNNSIAI | Spike | 97 |
| VAYSNNSIAIPTNFTISV | Spike | 98 |
| AIPTNFTISVTTEILPV | Spike | 99 |
| SVTTEILPVSMTKTSV | Spike | 100 |
| PVSMTKTSVDCTMYI | Spike | 101 |
| DCTMYICGDSTECSNLLL | Spike | 102 |
| DSTECSNLLLQYGSF | Spike | 103 |
| LLQYGSFCTQLNRALTGI | Spike | 104 |
| TQLNRALTGIAVEQDK | Spike | 105 |
| GIAVEQDKNTQEVFAQVK | Spike | 106 |
| NTQEVFAQVKQIYKTPPI | Spike | 107 |
| VKQIYKTPPIKDFGGFNF | Spike | 108 |
| PPIKDFGGFNFSQIL | Spike | 109 |
| NFSQILPDPSKPSKRSFI | Spike | 110 |
| PSKPSKRSFIEDLLFNKV | Spike | 111 |
| FIEDLLFNKVTLADAGFI | Spike | 112 |
| KVTLADAGFIKQYGDCL | Spike | 113 |
| FIKQYGDCLGDIAARDLI | Spike | 114 |
| LGDIAARDLICAQKFNGL | Spike | 115 |
| LICAQKFNGLTVLPPLL | Spike | 116 |
| GLTVLPPLLTDEMIAQY | Spike | 117 |
| LTDEMIAQYTSALLAGTI | Spike | 118 |
| YTSALLAGTITSGWTF | Spike | 119 |
| TITSGWTFGAGAALQIPF | Spike | 120 |
| GAGAALQIPFAMQMAYRF | Spike | 121 |
| PFAMQMAYRFNGIGV | Spike | 122 |
| RFNGIGVTQNVLYENQKL | Spike | 123 |
| QNVLYENQKLIANQF | Spike | 124 |
| KLIANQFNSAIGKIQDSL | Spike | 125 |
| SAIGKIQDSLSSTASAL | Spike | 126 |
| SLSSTASALGKLQDVV | Spike | 127 |
| LGKLQDVVNQNAQALNTL | Spike | 128 |
| NQNAQALNTLVKQLSSNF | Spike | 129 |
| TLVKQLSSNFGAISSVL | Spike | 130 |
| NFGAISSVLNDILSRL | Spike | 131 |
| LNDILSRLDPPEAEVQI | Spike | 132 |
| DPPEAEVQIDRLITGRL | Spike | 133 |
| IDRLITGRLQSLQTYV | Spike | 134 |
| LQSLQTYVTQQLIRAAEI | Spike | 135 |
| TQQLIRAAEIRASANL | Spike | 136 |
| EIRASANLAATKMSECVL | Spike | 137 |
| AATKMSECVLGQSKRVDF | Spike | 138 |
| VLGQSKRVDFCGKGYHLM | Spike | 139 |
| KRVDFCGKGYHLMSF | Spike | 140 |
| LMSFPQSAPHGVVFLHV | Spike | 141 |
| PHGVVFLHVTYVPAQEK | Spike | 142 |
| VTYVPAQEKNFTTAPAI | Spike | 143 |
| KNFTTAPAICHDGKAHF | Spike | 144 |
| ICHDGKAHFPREGVFV | Spike | 145 |
| FPREGVFVSNGTHWFV | Spike | 146 |
| SNGTHWFVTQRNFYEPQI | Spike | 147 |
| TQRNFYEPQIITTDNTFV | Spike | 148 |
| QIITTDNTFVSGNCDVVI | Spike | 149 |
| FVSGNCDVVIGIVNNTVY | Spike | 150 |
| VIGIVNNTVYDPLQPEL | Spike | 151 |
| VYDPLQPELDSFKEELDK | Spike | 152 |
| LDSFKEELDKYFKNHTSP | Spike | 153 |
| DKYFKNHTSPDVDLGDI | Spike | 154 |
| SPDVDLGDISGINASVV | Spike | 155 |
| ISGINASVVNIQKEIDRL | Spike | 156 |
| VNIQKEIDRLNEVAKNL | Spike | 157 |
| RLNEVAKNLNESLIDL | Spike | 158 |
| LNESLIDLQELGKYEQYI | Spike | 159 |
| QELGKYEQYIKWPWYIWL | Spike | 160 |
| YIKWPWYIWLGFIAGLI | Spike | 161 |
| WLGFIAGLIAIVMVTIML | Spike | 162 |
| GLIAIVMVTIMLCCM | Spike | 163 |
| TIMLCCMTSCCSCLK | Spike | 164 |
| CSCLKGCCSCGSCCKF | Spike | 165 |
| SCGSCCKFDEDDSEPVLK | Spike | 166 |
| DEDDSEPVLKGVKLHY | Spike | 167 |
| YSSGPHRLRENPP | +1FS spike | 168 |
| CSCSWCCCLWCPASV | +1FS spike | 169 |
| PPEHSCLQPTPTALPEAC | +1FS spike | 170 |
| PTPTALPEACTTPTR | +1FS spike | 171 |
| ACTTPTRCSDPACCTLPR | +1FS spike | 172 |
| SDPACCTLPRTCSCL | +1FS spike | 173 |
| PRTCSCLSSATPGSTPST | +1FS spike | 174 |
| SATPGSTPSTCPAPMAPR | +1FS spike | 175 |
| STCPAPMAPRDSTTPCCP | +1FS spike | 176 |
| PRDSTTPCCPSTTGCTL | +1FS spike | 177 |
| PCCPSTTGCTLPAPR | +1FS spike | 178 |
| LPAPRSPTSSEAGSSAP | +1FS spike | 179 |
| SSEAGSSAPHWTARPR | +1FS spike | 180 |
| AGSSAPHWTARPRAC | +1FS spike | 181 |
| TTPPTWSSKCASSSSATT | +1FS spike | 182 |
| SSKCASSSSATTPSW | +1FS spike | 183 |
| TTPSWASTTTRTTRAGWK | +1FS spike | 184 |
| TTRTTRAGWKASSGCTAA | +1FS spike | 185 |
| WKASSGCTAAPTTAPSST | +1FS spike | 186 |
| TAAPTTAPSSTCPSL | +1FS spike | 187 |
| WTWKASRATSRTCASSCL | +1FS spike | 188 |
| TSRTCASSCLRTSTATSR | +1FS spike | 189 |
| CLRTSTATSRSTASTPL | +1FS spike | 190 |
| SRSTASTPLSTSCGICLR | +1FS spike | 191 |
| LSTSCGICLRASLLWNPW | +1FS spike | 192 |
| GICLRASLLWNPWWVI | +1FS spike | 193 |
| PWWICPSASTSPGFRHCW | +1FS spike | 194 |
| ICPSASTSPGFRHCW | +1FS spike | 195 |
| HLAIAAADGQLVPPLTMW | +1FS spike | 196 |
| GQLVPPLTMWATCSL | +1FS spike | 197 |
| PPLTMWATCSLEPSC | +1FS spike | 198 |
| STTRTAPSPTPWIVLWIL | +1FS spike | 199 |
| SPSPWKRASTRPATSGC | +1FS spike | 200 |
| STRPATSGCSPPNPSC | +1FS spike | 201 |
| CSPPNPSCGSPISPI | +1FS spike | 202 |
| GSPISPICAPSARCSM | +1FS spike | 203 |
| CAPSARCSMPPDSPL | +1FS spike | 204 |
| PPDSPLCTPGTGSGSAI | +1FS spike | 205 |
| LCTPPGTGSGSAIAW | +1FS spike | 206 |
| IAWPTTPCCTTPPASAP | +1FS spike | 207 |
| CTTPPASAPSSATACPL | +1FS spike | 208 |
| TTCASQTCTPTA | +1FS spike | 209 |
| SGEMKCGRLPLDRQAR | +1FS spike | 210 |
| LPLDRQARSPTTTTSCPT | +1FS spike | 211 |
| SPTTTTSCPTTSPAV | +1FS spike | 212 |
| LPGTATTWTPKSAATTI | +1FS spike | 213 |
| TPKSAATTITCTGCSGSP | +1FS spike | 214 |
| AATTITCTGCSGSPI | +1FS spike | 215 |
| SPSSGTSPPRSIRPAAPL | +1FS spike | 216 |
| PRSIRPAAPLVTAWK | +1FS spike | 217 |
| PLVTAWKASTATSHCSPT | +1FS spike | 218 |
| STATSHCSPTAFSPQMAW | +1FS spike | 219 |
| PTAFSPQMAWAISPTEWW | +1FS spike | 220 |
| ASNCCMPLPQCAALRK | +1FS spike | 221 |
| CMPLPQCAALRKAPI | +1FS spike | 222 |
| QRATRSSCHSSSLAGI | +1FS spike | 223 |
| HSSSLAGISPIPQTPLEI | +1FS spike | 224 |
| SPIPQTPLEIPRHWKSW | +1FS spike | 225 |
| PLEIPRHWKSWTSPL | +1FS spike | 226 |
| WKSWTSPLAASAECL | +1FS spike | 227 |
| SPLAPTPAIRWQCCTR | +1FS spike | 228 |
| TVPKCPWPFTPI | +1FS spike | 229 |
| HLHGGCTPPAAMCFR | +1FS spike | 230 |
| CTPPAAMCFRPEPAV | +1FS spike | 231 |
| TIATSATSPSALESAPAT | +1FS spike | 232 |
| PSALESAPATRHRQTAL | +1FS spike | 233 |
| ATRHRQTALGEPEAWPAR | +1FS spike | 234 |
| LGEPEAWPARASLPTQCL | +1FS spike | 235 |
| ARASLPTQCLWAPRTAW | +1FS spike | 236 |
| CLWAPRTAWPTPTTLSL | +1FS spike | 237 |
| WPTPTTLSLSPPTSPSA | +1FS spike | 238 |
| PQRSCLCP | +1FS spike | 239 |
| PRPAWTAPCTSAAIPPSA | +1FS spike | 240 |
| CTSAAIPPSAPTCCCSTA | +1FS spike | 241 |
| PSAPTCCCSTAASAP | +1FS spike | 242 |
| QGSPWNRTRTPKRCSPK | +1FS spike | 243 |
| SRSTRPLLSRTSAASI | +1FS spike | 244 |
| SRTSAASISARFCPIL | +1FS spike | 245 |
| SARFCPILASPASGASSR | +1FS spike | 246 |
| ASPASGASSRTCCSTK | +1FS spike | 247 |
| HWPTPASSSSMAIVWATL | +1FS spike | 248 |
| SSSMAIVWATLPPGI | +1FS spike | 249 |
| SPSTHLPCWPAQSQAA | +1FS spike | 250 |
| WPAQSQAAGHLEQAPLCR | +1FS spike | 251 |
| GHLEQAPLCRSPLLCRW | +1FS spike | 252 |
| CRSPLLCRWPTGSTA | +1FS spike | 253 |
| PRMCCTRTR | +1FS spike | 254 |
| SPTSSTAPSARSR | +1FS spike | 255 |
| AAQQAPWESCRTWSTRM | +1FS spike | 256 |
| APWESCRTWSTRMPR | +1FS spike | 257 |
| TPWSSSCPPTSAPSAL | +1FS spike | 258 |
| ADWTLLRPRCRST | +1FS spike | 259 |
| SQADCRASRHT | +1FS spike | 260 |
| SEPPRLEPLPIWPPPRCL | +1FS spike | 261 |
| LPIWPPPRCLSVCWARAR | +1FS spike | 262 |
| CLSVCWARAREWTFAAR | +1FS spike | 263 |
| WARAREWTFAARATT | +1FS spike | 264 |
| ASLSLPLTAWCFCT | +1FS spike | 265 |
| HMCPLKRRISPPLQPSAT | +1FS spike | 266 |
| ISPPLQPSATTAKPTFL | +1FS spike | 267 |
| LQPSATTAKPTFLEK | +1FS spike | 268 |
| PTFLEKACSCPTAPI | +1FS spike | 269 |
| HSGTSTSPRSSPPTTPSC | +1FS spike | 270 |
| RSSPPTTPSCLATAT | +1FS spike | 271 |
| TIPCTTLCSPSWTASKR | +1FS spike | 272 |
| SPSWTASKRNWTSTLR | +1FS spike | 273 |
| RNWTSTLRTTQAPTWTW | +1FS spike | 274 |
| TTQAPTWTWAISAESM | +1FS spike | 275 |
| APTWTWAISAESMPA | +1FS spike | 276 |
| STCKNWGSTSSTSSGPGT | +1FS spike | 277 |
| TSSTSSGPGTSGWAL | +1FS spike | 278 |
| STSSGPGTSGWALSP | +1FS spike | 279 |
| WSQSCCVA | +1FS spike | 280 |
| RAVVAVAAAASSTRTIL | +1FS spike | 281 |
| VAAAASSTRTILSPC | +1FS spike | 282 |
| SSWYCMHAMLAAPFPSWV | +1FS spike | 283 |
| MLAAPFPSWVPRVSPDL | +1FS spike | 284 |
| WVPRVSPDLGSQVCSHL | +1FS spike | 285 |
| LGSQVCSHLHLPHSPPLL | +1FS spike | 286 |
| LHLPHSPPLLVPDTSQAR | +1FS spike | 287 |
| LLVPDTSQARSNAAQNA | +1FS spike | 288 |
| PLAINESLTKLY | +1FS spike | 289 |
| PQGWSISCQPHPGA | +1FS spike | 290 |
| GPAPFYPLEDGTAGEQLHK | 9935 | 291 |
| YGLNTNHR | 33451 | 293 |
| ELLNSMGISQPTVVFVSK | 6239 | 293 |
| KLPIIQK | 12509 | 294 |
| IIIMDSK | 15467 | 295 |
| TIALIMNSSGSTGLPK | 29537 | 296 |
| FEEELFLR | 7693 | 267 |
| YDLSNLHEIASGGAPLSK | 33224 | 298 |
| EVGEAVAK | 7351 | 299 |
| QGYGLTETTSAILITPEGDDKPGAVGK | 23417 | 300 |
| VVPFFEAK | 32478 | 301 |
| TLGVNQR | 29665 | 302 |
| GPMIMSGYVNNPEATNALIDK | 9994 | 303 |
| EIVDYVASQVTTAK | 6401 | 304 |
| GGVVFVDEVPK | 9405 | 305 |
| PPPX | wherein X is any nucleotide and PPP is a | 306 |
| | trinucleotide repeat of any nucleotide | |
| m1Ψm1Ψm1ΨX | wherein X is any nucleotide and m1Ψ is (N)1- | 307 |
| | methylpseudouridine | |
| CUUAGG | Slippery sequence | 308 |
| CUUGAC | Slippery sequence | 309 |
| CAGCAG | Slippery sequence | 310 |
| UCUGCGG | Slippery sequence | 311 |
| CCTATTTTTCGGCAA | WT Slippery sequence | 312 |
| CCTATTTTCCGGCAA | T187C Slippery sequence | 313 |
| TCACGGTTTTGGAAT | WT Slippery sequence | 314 |
| TCACGGTTCTGGAAT | T208C Slippery sequence | 315 |
| CTGCCTAAAGGTGTC | WT Slippery sequence | 316 |
| CTGCCTAAGGGTGTC | A206G Slippery sequence | 317 |
| KIMTSITR | +1 frameshift of encoded peptide of | 318 |
| | example 2 | |
| KIMTITR | Mutant +1 frameshift of encoded peptide of | 319 |
| | example 2 | |
| m1Ψcam1Ψgaacm1Ψccm1Ψcm1Ψggam1Ψcm1Ψa | Slippery Site A window-wherein m1Ψ is (N)1- | 320 |
| cm1Ψggm1Ψcm1Ψgccm1ΨAAAGgm1Ψgm1Ψcgc | methylpseudouridine | |
| m1Ψcm1Ψgccm1Ψcam1Ψagaacm1Ψgccm1Ψgcgm | | |
| 1Ψgagam1Ψm1Ψcm1Ψcgcam1Ψgccagagam1Ψcc | | |
| m1Ψam1Ψm1Ψm1Ψm1Ψm1Ψcggcaam1Ψcaaam1 | | |
| Ψcam1Ψm1Ψccggam1Ψacm1Ψgcgam1Ψm1Ψm1 | | |
| Ψm1Ψaagm1Ψgm1Ψm1Ψgm1Ψm1Ψccam1Ψm1Ψ | | |
| ccam1Ψcacggm1Ψm1Ψm1Ψm1Ψggaam1Ψgm1Ψ | | |
| m1Ψm1Ψacm1Ψacacm1Ψcggam1Ψm1Ψm1Ψm1 | | |
| Ψgam1Ψam1Ψgm1Ψggam1Ψm1Ψm1Ψcgagm1Ψc | | |
| gm1Ψcm1Ψm1Ψaam1Ψgm1Ψam1Ψagam1Ψm1Ψ | | |
| m1Ψgcgm1Ψgagam1Ψm1Ψcm1Ψcgcam1Ψgccaga | Slippery Site B window-wherein m1Ψ is (N)1- | 321 |
| gam1Ψccm1Ψam1Ψm1ΨM1ΨM1ΨM1ΨCggcaam1 | methylpseudouridine | |
| Ψcaaam1Ψcam1Ψm1Ψccggam1Ψacm1Ψgcgam1 | | |
| Ψm1Ψm1Ψm1Ψaagm1Ψgm1Ψm1Ψgm1Ψm1Ψcca | | |
| m1Ψm1Ψccam1Ψcacggm1Ψm1Ψm1Ψm1Ψggaam | | |
| 1Ψgm1Ψm1Ψm1Ψacm1Ψacacm1Ψcggam1Ψam1 | | |
| Ψm1Ψm1Ψgam1Ψam1Ψgm1Ψggam1Ψm1Ψm1Ψc | | |
| gagm1Ψcgm1Ψcm1Ψm1Ψaam1Ψgm1Ψam1Ψaga | | |
| m1Ψ'm1Ψ | | |
| m1Ψgcgm1Ψgagam1Ψm1Ψcm1Ψcgcam1Ψgccaga | Slippery Site C window-wherein m1Ψ is (N)1- | 322 |
| gam1Ψccm1Ψam1Ψm1Ψm1Ψm1Ψm1Ψcggcaam1 | methylpseudouridine | |
| Ψcaaam1Ψcam1Ψm1Ψccggam1Ψacm1Ψgcgam1 | | |
| Ψm1Ψm1Ψm1Ψaagm1Ψgm1Ψm1Ψgm1Ψm1Ψcca | | |
| m1Ψm1Ψccam1ΨcacggM1ΨM1ΨM1ΨM1Ψggaam | | |
| 1Ψgm1Ψm1Ψm1Ψacm1Ψacacm1Ψcggam1Ψam1 | | |
| Ψm1Ψm1Ψgam1Ψam1Ψgm1Ψggam1Ψm1Ψm1Ψc | | |
| gagm1Ψcgm1Ψcm1Ψm1Ψaam1Ψgm1Ψam1Ψaga | | |
| m1Ψm1Ψ | | |
| ACCCGGm1Ψm1Ψm1ΨCAGACA | SpikeFS1-wherein m1Ψ is (N)1- | 323 |
| | methylpseudouridine | |
| TACGGCm1Ψm1Ψm1ΨCAGCCC | SpikeFS2-wherein m1Ψ is (N)1- | 324 |
| | methylpseudouridine | |
| AATGTGm1Ψm1Ψm1ΨCAGACC | SpikeFS2-wherein m1Ψ is (N)1- | 325 |
| | methylpseudouridine | |
| GCCCACm1Ψm1Ψm1ΨCCTAGA | SpikeFS3-wherein m1Ψ is (N)1- | 326 |
| | methylpseudouridine | |
| GTGGACm1Ψm1Ψm1Ψm1ΨGCGGC | SpikeFS4-wherein m1Ψ is (N)1- | 327 |
| | methylpseudouridine | |
| GTGGTGm1Ψm1Ψm1ΨCTGCAC | SpikeFS5-wherein m1Ψ is (N)1- | 328 |
| | methylpseudouridine | |
| TGCCCCm1Ψm1Ψm1ΨCCCGTC | SpikeSX-wherein m1Ψ is (N)1- | 329 |
| | methylpseudouridine | |
| aaagatcatgacatcgattacaagg | WT | 330 |
| aaagatcatgacatAgattacaagg | Mutant | 331 |

### TABLES

TABLE 1
LC-MS/MS analysis of Fluc +1FS high-molecular weight polypeptide.

| | | | | | | | | | | | | | Perc- | | | | |
|---|---|---|---|---|---|---|---|---|---|---|---|---|---|---|---|---|---|
| | | | | | | | | | | | | Con- | olator | Perc- | | | |
| | | | | | | | | | | | | fidence | q-Value | olator | XCorr | | |
| | | | | | | | | Num- | | Number | | by | by | PEP by | by | Top | |
| | | | | | | | | ber | Num- | of | Theo | Search | Search | Search | Search | Apex | |
| | | Con- | | SEQ | Modi- | Qva | Qvality | of | ber | Missed | MHplus | Engine | Engine | Engine | Engine | RT | |
| Group | Che- | fidence | | ID | fi- | lity | q- | Pro- | of | Cleav- | in | Sequest | Sequest | Sequest | Sequest | in | |
| ID | cked | | Sequence | NO | cations | PEP | value | teins | PSMs | ages | Da | HT | HT | HT | HT | min | Frame |
| 9935 | TRUE | High | GPAPFYPLEDG | 291 | | 0. | 0. | 2 | 2 | 0 | 2026. | High | 0. | 5.42E− | 2.02 | 39.85 | in- |
| | | | TAGEQLHK | | | 000143 | 00082897 | | | | 98688 | | 0007087 | 05 | | | frame |
| 33451 | TRUE | High | YGLNTNHR | | | 0. | 0. | 2 | 2 | 0 | 974. | High | 0. | 0. | 1.54 | 17.78 | in- |
| | | | | | | 006271 | 00082897 | | | | 48025 | | 0007081 | 003422 | | | frame |
| 6239 | TRUE | High | ELLNSMGISQP | | 1xOxi | 0. | 0. | 2 | 1 | 0 | 1965. | High | 0. | 0. | 1.62 | 46.54 | in- |
| | | | TVVFVSK | | dation | 003792 | 00082897 | | | | 03614 | | 0007087 | 001972 | | | frame |
| | | | | | [M6] | | | | | | | | | | | | |
| 12509 | TRUE | High | KLPIIQK | | | 0. | 0. | 3 | 2 | 1 | 839. | High | 0. | 0. | 1.43 | 25.88 | in- |
| | | | | | | 106022 | 00980118 | | | | 5713 | | 007388 | 07696 | | | frame |
| 15467 | TRUE | High | IIIMDSK | | 1xOxi | 0. | 0. | 2 | 3 | 0 | 835. | High | 0. | 0. | 1.72 | 23.99 | in- |
| | | | | | dation | 052499 | 00363672 | | | | 45937 | | 002833 | 03515 | | | frame |
| | | | | | [M4] | | | | | | | | | | | | |
| 29537 | TRUE | High | TIALIMNSSGS | | 1xOxi | 0. | 0. | 2 | 1 | 0 | 1605. | High | 0. | 0. | 2.15 | 38.1 | in- |
| | | | TGLPK | | dation | 001001 | 00082897 | | | | 85163 | | 0007087 | 000459 | | | frame |
| | | | | | [M6] | | | | | | | | | | | | |
| 7693 | TRUE | High | FEEELFLR | | | 0. | 0. | 2 | 2 | 0 | 1082. | High | 0. | 0. | 2.31 | 43.71 | +1 FS |
| | | | | | | 027871 | 00082897 | | | | 55169 | | 0007087 | 01757 | | | |
| 33224 | TRUE | High | YDLSNLHEIAS | | | 9.19E− | 0. | 2 | 2 | 0 | 1871. | High | 0. | 2.68E− | 2.98 | 41.34 | +1 FS |
| | | | GGAPLSK | | | 06 | 00082897 | | | | 94977 | | 0007087 | 06 | | | |
| 7351 | TRUE | High | EVGEAVAK | | | 0. | 0. | 2 | 1 | 0 | 802. | High | 0. | 0. | 0.94 | 17.65 | +1 FS |
| | | | | | | 017231 | 00082897 | | | | 43051 | | 0007087 | 01038 | | | |
| 23417 | TRUE | High | QGYGLTETTSA | | | 0. | 0. | 2 | 1 | 0 | 2718. | High | 0. | 0.01 | 1.04 | 42.98 | +1 FS |
| | | | ILITPEGDDKP | | | 021509 | 00082897 | | | | 38323 | | 0007087 | 322 | | | |
| | | | GAVGK | | | | | | | | | | | | | | |
| 32478 | TRUE | High | VVPFFEAK | | | 0. | 0. | 2 | 1 | 0 | 936. | High | 0. | 0. | 1.62 | 39.85 | +1 FS |
| | | | | | | 091782 | 00908456 | | | | 15893 | | 006893 | 06561 | | | |
| 29665 | TRUE | High | TLGVNQR | | | 0. | 0. | 2 | 1 | 0 | 787. | High | 0. | 0. | 1.05 | 19.53 | +1 FS |
| | | | | | | 020576 | 00082897 | | | | 44208 | | 0007087 | 01259 | | | |
| 9994 | TRUE | High | GPMIMSGYVNN | | 2xOxi | 4.34E− | 0. | 2 | 2 | 0 | 2267. | High | 0. | 9.50E− | 3.74 | 39.39 | +1 FS |
| | | | PEATNALIDK | | dation | 07 | 00082897 | | | | 06824 | | 0007087 | 08 | | | |
| | | | | | [M3; | | | | | | | | | | | | |
| | | | | | M5] | | | | | | | | | | | | |
| 6401 | TRUE | High | EIVDYVASQVT | | | 0. | 0. | 2 | 2 | 0 | 1523. | High | 0. | 4.02E− | 2.52 | 41.76 | +1 FS |
| | | | TAK | | | 000109 | 00082897 | | | | 79516 | | 0007087 | 05 | | | |
| 9405 | TRUE | High | GGVVFVDEVPK | | | 0. | 0. | 2 | 2 | 0 | 1145. | High | 0. | 0. | 2.87 | 38.97 | +1 FS |
| | | | | | | 003535 | 00082897 | | | | 6201 | | 0007087 | 001817 | | | |

TABLE 2
Insertion and Deletion Frequencies in unmodified and
1-methylΨ mRNAs - Comparison of single nucleotide
deletion frequency or insertion frequency, normalised
to total reads (%), for unmodified or 1-methylΨ mRNA
(Welch's unpaired two-tailed T-test).

| Sample | Mean | | S.D. | |
|---|---|---|---|---|
| group | Deletions | Insertions | Deletions | Insertions |
| unmodified | 0.000862 | 0.000832 | 0.000118 | 0.000079 |
| 1-methylΨ | 0.000625 | 0.000848 | 0.00008 | 0.000263 |
| P-value | 0.05297 | 0.9311 | | |

1. A modified therapeutic mRNA comprising at least one frameshifting nucleic acid sequence that increases frequency of out-of-frame translation of the mRNA, wherein the at least one frameshifting nucleic acid sequence comprises at least one synonymous mutation for reducing frequency of out-of-frame translation of the modified therapeutic mRNA.

2. A modified therapeutic mRNA comprising at least one ribosomal slippery sequence that increases frequency of out-of-frame translation of the mRNA, wherein the at least one ribosomal slippery sequences comprises at least one synonymous mutation for reducing frequency of out-of-frame translation of the modified therapeutic mRNA.

3. A modified therapeutic mRNA comprising at least one at least one alternative reading frame sequence which encodes an alternative translation product that differs from the translation product of in-frame translation of the modified therapeutic mRNA and the modified therapeutic mRNA comprising at least one synonymous mutation for introducing a premature termination codon (PTC) in the at least one alternative reading frame sequence.

4. A method of producing a modified therapeutic mRNA having a reduced frequency of out-of-frame translation, the method comprising:
a. providing a therapeutic mRNA or a nucleic acid encoding the therapeutic mRNA;
b. identifying at least one frameshift sequence within the nucleic acid sequence of the therapeutic mRNA or a nucleic acid encoding the therapeutic mRNA;
c. producing a modified therapeutic mRNA comprising a modified at least one frameshift sequence, wherein the modified at least one frameshift sequence comprises at least one synonymous mutation.

5. A method of reducing off-target immunogenicity to a therapeutic mRNA and/or translation product thereof, the method comprising:
a. providing a therapeutic mRNA or a nucleic acid encoding the therapeutic mRNA;
b. identifying at least one frameshift sequence within the nucleic acid sequence of the therapeutic mRNA or a nucleic acid encoding the therapeutic mRNA;
c. producing a modified therapeutic mRNA comprising a modified at least one frameshift sequence, wherein the modified at least one frameshift sequence comprises at least one synonymous mutation; and
d. administering the modified therapeutic mRNA to a subject in need thereof.

6. A method of reducing out-of-frame translation of a therapeutic mRNA and/or increasing translation fidelity of a therapeutic mRNA, the method comprising:
a. providing a therapeutic mRNA or a nucleic acid encoding the therapeutic mRNA;
a. identifying at least one frameshift sequence within the nucleic acid sequence of the therapeutic mRNA or a nucleic acid encoding the therapeutic mRNA;
b. producing a modified therapeutic mRNA comprising a modified at least one frameshift sequence, wherein the modified at least one frameshift sequence comprises at least one synonymous mutation; and
c. translating the modified therapeutic mRNA.

7. The modified therapeutic mRNA of any of claims 1 to 3 or the method of any of claims 4 to 6, wherein the at least one synonymous mutation increases fidelity of translation of the modified therapeutic mRNA when in use.

8. The modified therapeutic mRNA of any of claims 1 to 3 and 7 or the method of any of claims 4 to 7, wherein the out-of-frame translation comprises a +1 frameshift, +2 frameshift, −1 frameshift, or −2 frameshift.

9. The modified therapeutic mRNA of any of claims 1 to 3, 7 and 8 or the method of any of claims 4 to 8, wherein the at least one synonymous mutation reduces off-target immunogenicity of the modified therapeutic mRNA and/or translation product thereof.

10. The modified therapeutic mRNA of any of claims 1 to 3 and 7 to 9 or the method of any of claims 4 to 9, wherein the at least one synonymous mutation mutates an out-of-frame codon to a non-cognate amino acid.

11. The modified therapeutic mRNA of any of claims 1 to 3 and 7 to 10 or the method of any of claims 4 to 10, wherein the off-target immunogenicity comprises cellular immunogenicity.

12. The modified therapeutic mRNA of any of claims 1 to 3 and 7 to 11 or the method of any of claims 4 to 11, wherein the at least one frameshifting nucleic acid sequence causes ribosome stalling.

13. The modified therapeutic mRNA of any of claims 1 to 3 and 7 to 12 or the method of any of claims 4 to 13, wherein the at least one frameshifting nucleic acid sequence comprises or further comprises at least one ribosomal slippery sequence.

14. The modified therapeutic mRNA or method of claim 13, wherein the at least one ribosomal slippery sequence comprises at least one of the sequences selected from:
a. XXXYYYZ, wherein X is any nucleotide, wherein Y is A or U, and wherein Z is A, U, or C;
b. PPPX, wherein X is any nucleotide and PPP is a trinucleotide repeat of any nucleotide;
c. m1Ψm1Ψm1ΨX, wherein X is any nucleotide and m1Ψ is (N)1-methylpseudouridine;
d. CUUAGG, CUUGAC, CAGCAG, or UCUGCGG; and/or
e. any one of (a) to (d) and sequences or formulae containing non-canonical nucleotides.

15. The modified therapeutic mRNA of any of claims 1, 2 and 7 to 12 or the method of any of claims 4 to 14, wherein the at least one frameshifting nucleic acid sequence comprises or further comprises at least one alternative reading frame sequence which encodes an out-of-frame product that differs from the translation product of in-frame translation of the modified therapeutic mRNA.

16. The modified therapeutic mRNA or method of claim 15, wherein the at least one synonymous mutation comprises a synonymous mutation for introducing a premature termination codon (PTC) in the one alternative reading frame sequence.

17. The modified therapeutic mRNA of any of claims 1 to 3 and 7 to 16 or the method of any of claims 4 to 16, wherein the modified therapeutic mRNA comprises at least one chemically modified ribonucleotide.

18. The modified therapeutic mRNA or method of claim 17, wherein the at least one chemically modified ribonucleotide comprises (N)1-methylpseudouridine.

19. A nucleic acid encoding a modified therapeutic mRNA of any preceding claim;
optionally wherein the nucleic acid comprises a DNA template for in vitro transcription of the modified therapeutic mRNA.

20. A modified therapeutic mRNA according to any of claims 1 to 18 for use as a medicament; optionally for use as a vaccine.

21. The method of any of claims 6 to 18, wherein translating comprises translating in vivo or in vitro.

22. The method of any of claims 6 to 18, wherein in vivo translating comprises administering the modified therapeutic mRNA to a subject in need thereof.

23. The method of any of claims 4 to 18 and 21 to 22, wherein identifying comprises sequencing the modified therapeutic mRNA and/or the nucleic acid encoding the modified therapeutic mRNA; and/or
analysing the modified therapeutic mRNA sequence and/or the nucleic acid encoding the modified therapeutic mRNA sequence.
